(19) United States
(12) Patent Application Publication
(10) Pub. No.: US 2025/0032566 A1
(43) Pub. Date: Jan. 30, 2025

(54) GENETICALLY MODIFIED PHAGES AND USES THEREOF

(71) Applicant: Yeda Research and Development Co. Ltd., Rehovot (IL)

(72) Inventors: Rotem SOREK, Rehovot (IL); Erez YIRMIYA, Rehovot (IL); Azita LEAVITT, Rehovot (IL); Gil AMITAI, Rehovot (IL); Jeremy Elliot GARB, Rehovot (IL)

(73) Assignee: Yeda Research and Development Co. Ltd., Rehovot (IL)

(21) Appl. No.: 18/713,665

(22) PCT Filed: Dec. 5, 2022
(86) PCT No.: PCT/IL2022/051292

§ 371 Date: May 27, 2024

(30) Foreign Application Priority Data
Dec. 5, 2021 (IL) 288680

(51) Int. Cl. A61K 35/76; C12N 7/00

(52) U.S. Cl. CPC A61K 35/76; C12N 7/00; C12N 2795/10022; C12N 2795/10033; C12N 2795/10052

(57) ABSTRACT

Genetically modified phages are provided. Accordingly, there is provided a genetically modified phage comprising a polynucleotide encoding an anti-defense system polypeptide. Also provided are methods of producing and using same.

### RELATED APPLICATION/S

This application claims the benefit of priority of Israeli Patent Application No. 288680 filed on Dec. 5, 2021, the contents of which are incorporated herein by reference in their entirety.

### SEQUENCE LISTING STATEMENT

The file entitled 94587.xml, created on Dec. 5, 2022, comprising 2,071,571 bytes, submitted concurrently with the filing of this application is incorporated herein by reference.

### FIELD AND BACKGROUND OF THE INVENTION

The present invention, in some embodiments thereof, relates to genetically modified phages and used thereof.

The ongoing arms race between prokaryotes and the viruses that infect them, bacteriophages (phages), has led to the continuous and intensive evolution of efficient resistance systems to protect prokaryotes from phage infection. Bacterial and archaeal genomes can contain many different kinds of anti-phage defense systems, which can be clustered in genomic islands termed defense islands [Makarova et al. J Bacteriol. 2011 November; 193(21): 6039-6056; Doron et al. Science 2018 359(6379):eaar4120]. Typically, prokaryotes defense systems target one or more of the stages of phage infection in order to thwart the attack, and are rapidly evolving and diverging to answer the fast evolutionary response of phages to these defense strategies [Bernheim and Sorek Nat Rev Microbiol. (2020) 18(2):113-119 and Labrie et al Nature Reviews Microbiology (2010) 8, 317-327].

Whereas early research focused on restriction-modification (R-M) and later on CRISPR-Cas [Labrie et al Nature Reviews Microbiology 8, 317-327 (2010)] as the main mechanisms of defense against phage, recent studies exposed dozens of previously unknown defense systems that are widespread among bacteria and archaea [see e.g. Bernheim, A. & Sorek, R. Nat. Rev. Microbiol. 18, 113-119 (2020)]; among them the BREX system [Goldfarb et al. EMBO J. (2015) 34, 169-83; and International Application Publication No. WO2015/059690], the DISARM system [International Application Publication No. WO2018142416], and the Thoeris, Hachiman, Gabija, Septu and Lamassu systems [Doron, S. et al. Science 359, eaar4120 (2018); and International Application Publication No. WO2018/220616].

On the counter arm, phages developed mechanisms that allow them to overcome bacterial defenses [see e.g. Samson, J. E., et al. Nat. Rev. Microbiol. 11, 675-687 (2013); and Ofir, G. & Sorek, R. Cell 172, 1260-1270 (2018)]. Multiple phages were shown to encode anti-restriction proteins, which inhibit restriction-modification (R-M) systems by direct binding to the restriction enzyme, masking restriction sites, or degrading cofactors important for R-M activity [see e.g. Atanasiu, C. Nucleic Acids Res. 30, 3936-3944 (2002); Walkinshaw, M. et al. Mol. Cell 9, 187-194 (2002); Iida, S., et al. Virology 157, 156-166 (1987); Drozdz, M., et al. Nucleic Acids Res. 40, 2119-2130 (2012); Studier, F. W. & Movva, N. R. J. Virol. 19, 136-145 (1976)]. Phages are also known to encode many CRISPR-Cas inhibitors, which function via a variety of mechanisms that include inhibition of CRISPR RNA loading, induction of non-specific DNA-binding by the CRISPR-Cas complex, prevention of target DNA binding or cleavage, and many additional mechanisms [Thavalingam, A. et al. Nat. Commun. 10, 2806 (2019); Lu, W.-T., et al. Nucleic Acids Res. 49, 3381-3393 (2021); Bondy-Denomy, J. et al. Nature 526, 136-139 (2015); Stanley, S. Y. & Maxwell, K. L. Annu. Rev. Genet. 52, 445-464 (2018); Li, Y. & Bondy-Denomy, J. Cell Host Microbe 29, 704-714 (2021); and Jia, N. & Patel, D. J. Nat. Rev. Mol. Cell Biol. 22, 563-579 (2021)]. Phage proteins and non-coding RNAs that inhibit toxin-antitoxin-mediated defense have also been described [Otsuka, Y. & Yonesaki, T. Mol. Microbiol. 83, 669-681 (2012); and Blower, T. R., et al. PLoS Genet. 8, e1003023 (2012)].

Harnessing phages and their defense mechanisms as anti-bacterial agents for therapeutic uses has gained much interest over the last decade, especially in light of the substantial rise in the prevalence of bacterial antibiotic resistance, coupled with an inadequate number of new antibiotics (see e.g. Gibb et al. Pharmaceuticals 2021, 14, 634).

### SUMMARY OF THE INVENTION

According to an aspect of some embodiments of the present invention there is provided a genetically modified phage comprising a polynucleotide encoding an anti-defense system polypeptide.

According to some embodiments of the invention, the anti-defense system polypeptide is selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR; and
(v) a TAD2 polypeptide having at least 80% identity and/or an e-value≤0.05 to an amino acid sequence selected from the group consisting of SEQ ID NO: 1046-1295, and wherein expression of the TAD2 polypeptide in a B. subtilis BEST7003 comprising SEQ ID NO: 741 or 1300 increases sensitivity of the B. subtilis BEST7003 to infection by at least one B. subtilis phage selected from the group consisting of SPO1L1, SPO1L2, SPO1L4 and SPO1L5.

According to some embodiments of the invention, the anti-defense system polypeptide is selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8; and
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR.

According to some embodiments of the invention, the genetically modified phage, having an increased infectivity to at least one bacteria as compared to a non-genetically modified phage of the same species.

According to some embodiments of the invention, the polynucleotide is heterologous to the phage.

According to some embodiments of the invention, the phage comprises genomic segments of a distinct phage integrated in a genome of the phage.

According to an aspect of some embodiments of the present invention there is provided a nucleic acid construct comprising a polynucleotide encoding an anti-defense polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR; and
(v) a TAD2 polypeptide having at least 80% identity and/or an e-value≤0.05 to an amino acid sequence selected from the group consisting of SEQ ID NO: 1046-1295, and wherein expression of the TAD2 polypeptide in a B. subtilis BEST7003 comprising SEQ ID NO: 741 or 1300 increases sensitivity of the B. subtilis BEST7003 to infection by at least one B. subtilis phage selected from the group consisting of SPO1L1, SPO1L2, SPO1L4 and SPO1L5; and a nucleic acid sequence heterologous to the polynucleotide which facilitates expression and/or integration of the polynucleotide in a phage genome.

According to an aspect of some embodiments of the present invention there is provided a nucleic acid construct comprising a polynucleotide encoding an anti-defense polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8; and
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR;
   and a nucleic acid sequence heterologous to the polynucleotide which facilitates expression and/or integration of the polynucleotide in a phage genome.

According to some embodiments of the invention, the nucleic acid sequence is selected from the group consisting of: a promoter, a recombination element, an element for expression of multiple polynucleotides from a single construct, a transmissible element and a selectable marker.

According to some embodiments of the invention, the at least 80% is at least 90%.

According to some embodiments of the invention, the polynucleotide comprises the SEQ ID NO.

According to some embodiments of the invention:
the amino acid sequence of (i) is selected from the group consisting of SEQ ID NO: 22, 324, 337, 338, 345, 400, 404, 438, 460, 464 and 543;
the amino acid sequence of (ii) is selected from the group consisting of SEQ ID NO: 32, 598, 604, 607 and 609;
the amino acid sequence of (iii) is selected from the group consisting of SEQ ID NO: 33, 689, 723, 725 and 726; and/or the amino acid sequence of (v) is selected from the group consisting of SEQ ID NO: 1046-1051.

According to some embodiments of the invention:
the amino acid sequence of (i) is selected from the group consisting of SEQ ID NO: 22, 324, 337, 338, 345, 400, 404, 438, 460, 464 and 543;
the amino acid sequence of (ii) is selected from the group consisting of SEQ ID NO: 32, 598, 604, 607 and 609; and/or
the amino acid sequence of (iii) is selected from the group consisting of SEQ ID NO: 33, 689, 723, 725 and 726.

According to an aspect of some embodiments of the present invention there is provided a method of producing a phage, the method comprising contacting the phage with the nucleic acid construct, under conditions which allow integration of the polynucleotide in a genome of the phage, thereby producing the phage.

According to some embodiments of the invention, the phage does not endogenously express the anti-defense system polypeptide.

According to an aspect of some embodiments of the present invention there is provided a method of infecting a bacteria, the method comprising contacting the bacteria with the genetically modified phage, thereby infecting the bacteria.

According to some embodiments of the invention, the contacting is effected in-vitro or ex-vivo.

According to some embodiments of the invention, the contacting is effected in-vivo.

According to an aspect of some embodiments of the present invention there is provided a method of treating a bacterial infection in a subject in need thereof, the method comprising administering to the subject a therapeutically effective amount of a phage comprising a polynucleotide encoding an anti-defense system polypeptide, thereby treating the bacterial infection in the subject.

According to some embodiments of the invention, the method comprising administering to the subject a therapeutically effective amount of an antibiotic.

According to an aspect of some embodiments of the present invention there is provided a phage comprising a polynucleotide encoding an anti-defense system polypeptide, for use in treating a bacterial infection in the subject in need thereof.

According to some embodiments of the invention, the phage further comprising an antibiotic.

According to an aspect of some embodiments of the present invention there is provided an article of manufacture comprising as active ingredients a phage comprising a polynucleotide encoding an anti-defense system polypeptide; and an antibiotic.

According to some embodiments of the invention, the phage and the antibiotic are in separate formulations.

According to some embodiments of the invention, the phage and the antibiotic are in a co-formulation.

According to some embodiments of the invention, the anti-defense system polypeptide is selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR; and
(v) a TAD2 polypeptide having at least 80% identity and/or an e-value≤0.05 to an amino acid sequence selected from the group consisting of SEQ ID NO: 1046-1295, and wherein expression of the TAD2 polypeptide in a B. subtilis BEST7003 comprising SEQ ID NO: 741 or 1300 increases sensitivity of the B. subtilis BEST7003 to infection by at least one B. subtilis phage selected from the group consisting of SPO1L1, SPO1L2, SPO1L4 and SPO1L5.

According to some embodiments of the invention, the anti-defense polypeptide is selected from the group consisting of:
(i) a TAD1 polypeptide having at least 80% identity and/or an e-value≤0.05 to an amino acid sequence selected from the group consisting of SEQ ID NO: 22 and 323-577, and wherein expression of the TAD1 polypeptide in a B. subtilis BEST7003 comprising SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of the HAD1 polypeptide in a B. subtilis BEST7003 comprising SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of the GAD1 polypeptide in a B. subtilis BEST7003 comprising SEQ ID NO: 740 increases sensitivity of the B. subtilis BEST7003 to infection by at least one B. subtilis phage selected from the group consisting of SpBeta, SpBetaL6, SpBetaL7, SpBetaL8; and
(iv) a DSAD1 polypeptide having at least 80% identity and/or an e-value≤0.05 to SEQ ID NO: 739, and wherein expression of the DSAD1 polypeptide in a B. subtilis BEST7003 comprising SEQ ID NO: 746 increases sensitivity of the B. subtilis BEST7003 to infection by a B. subtilis phage SPR.

According to some embodiments of the invention, the phage is the genetically modified phage.

According to some embodiments of the invention, the bacteria is selected from the group consisting of E. coli, P. aeruginosa, K. pneumoniae and C. difficile.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C demonstrate the principles of identification of anti-defense genes based on phage infection and genome analyses. FIG. 1A is a schematic flowchart of the experimental and analytical protocols used. FIG. 1B shows genome comparison of eight phages from the SBSphiJ group. Sequence similarity between genes is marked by grey shading. FIG. 1C shows infection profile of SBSphiJ-like and SpBeta-like phages infecting five Bacillus subtilis strains that express each of the defense systems Thoeris (specifically, from Bacillus cereus MSX-D12), Hachiman, Gabija, Septu and Lamassu. Fold defense was measured using serial dilution plaque assays, comparing the efficiency of plating (EOP) of phages on the system-containing strain to the EOP on a control strain that lacks the systems and contains an empty vector instead. Data represent an average of three replicates. Detailed data from individual plaque assays is found in FIGS. 10A-B.

FIGS. 2A-E demonstrate that TAD1 proteins inhibit Thoeris defense system. FIG. 2A is a schematic representation of the TAD1 locus in SBSphiJ7. Shown in the locus for two other phages, SBSphi3 and SBSphi4, in which endogenous TAD1 is missing. The coordinates of the presented locus within the phage genome are indicated below the name of each phage. FIG. 2B demonstrates that TAD1 cancels Thoeris-mediated defense. Results of phage infection experiments with eight phages of the SBSphiJ family. Data represent plaque-forming units per ml (PFU/ml) of phages infecting control cells (“no system”), cells expressing the Thoeris system (“Thoeris”), and cells expressing the Thoeris system and the TAD1 gene from SBSphiJ7 (“Thoeris+TAD1”). Nucleic acid and amino acid sequences of Thoeris are provided in SEQ ID NO: 741 and 1296-1297, nucleic acid and amino acid sequences of TAD1 are provided in SEQ ID Nos: 5 and 22. All phages except for SBSphiJ7 lack an endogenous TAD1. Data for phage SBSphiJ7 in which TAD1 was engineered under the control of its native promoter are also presented. Shown is the average of three replicates, with individual data points overlaid. FIG. 2C shows that TAD1 knockdown cancels anti-Thoeris activity. Results of phage SBSphiJ7 infection experiments. Data represent plaque-forming units per ml (PFU/ml) of SBSphiJ7 infecting cells expressing Thoeris and a dCAS9 system targeting TAD1, as well as control cells. Shown is the average of three replicates, with individual data points overlaid. FIG. 2D shows phylogenetic analysis of TAD1 homologs in phage and prophage genomes. The names of bacteria in which TAD1 homologs were found in prophages and tested experimentally are indicated on the tree. FIG. 2E demonstrates that TAD1 homologs (nucleic acid sequences of the TAD1 homologs are provided in SEQ ID NOs: 49, 94 and 188, amino acid sequences of the TAD1 homologs are provided in SEQ ID Nos: 438, 460 and 464) found in multiple prophages can inhibit the Thoeris system in B. subtilis. Results are representative of three replicates.

FIGS. 3A-D demonstrate that HAD1 proteins inhibit the Hachiman defense system. FIG. 3A is a schematic representation of the comparison of the genomic locus of HAD1 between SBSphiJ4 (contains an endogenous HAD1); and SBSphiJ5 and SBSphiJ7 (lack endogenous HAD1). FIG. 3B demonstrates that HAD1 cancels Hachiman-mediated defense. Results of phage infection experiments with eight phages of the SBSphiJ family. Data represent plaque-forming units per ml (PFU/ml) of phages infecting control cells (“no system”), cells expressing the Hachiman system (“Hachimen”), and cells expressing a Hachiman system and the HAD1 gene from SBSphiJ4 (“Hachimen+HAD1”). Nucleic acid and amino acid sequences of Hachimen are provided in SEQ ID NO: 742 and 1298-1299, nucleic acid and amino acid sequences of HAD1 are provided in SEQ ID Nos: 15 and 32. All phages except for SBSphiJ4 lack endogenous HAD1. Shown is the average of three replicates, with individual data points overlaid. FIG. 3C shows that HAD1 knockdown cancels anti-Hachiman activity. Results of phage SBSphiJ4 infection experiments. Data represent plaque-forming units per ml (PFU/ml) of SBSphiJ4 infecting cells expressing Hachiman and a dCAS9 system targeting HAD1, as well as control cells. Shown is the average of three replicates, with individual data points overlaid. FIG. 3D shows cladogram and multiple sequence alignment of HAD1 homologs. Conserved residues are colored in grey.

FIGS. 3E-G demonstrate that GAD1 proteins inhibit Gabija defense system. FIG. 3E is a schematic representation of the genomic locus of the anti-Gabija gene GAD1 (nucleic acid sequence provided in SEQ ID NO: 16, amino acid sequence provided in SEQ ID NO: 33) in multiple phages of the SpBeta group. FIG. 3F demonstrates that deletion of GAD1 from phage phi3T eliminates the ability of the phage to overcome the Gabija-mediated defense. FIG. 3G shows phylogeny and distribution of GAD1 homologs.

FIGS. 4A-D demonstrate that TAD1 cancels Thoeris-mediated defense by physically binding and sequestering the Thoeris-derived signaling molecule. FIG. 4A demonstrates that filtered lysates derived from cells expressing ThsB and TAD1 fail to activate ThsA in vitro. Results show NADase activity of purified ThsA protein incubated with filtered lysates derived from control cells, ThsB-expressing cells, or cells expressing both ThsB and TAD1, that were infected by phage SBSphiJ at MOI of 5. Lysates were collected at multiple time points following infection. NADase activity was calculated as the rate of change in εNAD+ fluorescence, due to εNAD+ hydrolysis, during the linear phase of the reaction. Bars represent the mean of three experiments, with individual data points overlaid. FIG. 4B demonstrates that the signaling molecule (v-cADPR) is eliminated by TAD1 in-vivo. Results show the peak (intensity) of the v-cADPR measured in LC-MS analysis of filtrates derived from cells right before and 120 minutes after infection with phage SBSphiJ. FIG. 4C demonstrate that TAD1 eliminate the signaling molecule rather than inhibiting its' production. Results show NADase activity of purified ThsA protein incubated with filtered lysates derived from ThsB-expressing cells 120 minutes post infection with phage SBSphiJ. Filtered lysates were either pre-incubated with TAD1 for 10 minutes in vitro (“with TAD1”) or with buffer (“W/O TAD1”). Control are filtered lysates from infected cells not expressing ThsB. NADase activity calculated and presented as in panel A. FIG. 4D demonstrates that TAD1 cancels Thoeris-mediated defense by binding to the signaling molecule rather than degrading it. Results show NADase activity of purified ThsA protein incubated with filtered lysates derived from infected cells expressing ThsB, that were additionally pre-incubated with TAD1 in vitro for 10 minutes, followed by an additional incubation of 5 minutes at either 25° C. or 85° C. (denaturing conditions). “With TAD1”, lysates pre-incubated with TAD1. “W/O TAD1”, lysates incubated by buffer instead of TAD1. “Control”, lysates derived from infected cells that do not express ThsB.

FIGS. 5A-E demonstrate that DSR2 defense system protects bacteria via depletion of NAD⁺. FIG. 5A is a schematic representation of domain organization of a DSR2 gene from B. subtilis 29R. Protein accession in NCBI is indicated above the gene. FIG. 5B demonstrates the efficiency of plating for the indicated three phages infecting a control B. subtilis strain BEST7003 which has an empty vector (“no system”) as compared to B. subtilis strain BEST7003 with cloned wild type DSR2 from B. subtilis 29R (“DSR2”) or the indicated mutant DSR2 versions. The Data represent plaque-forming units (PFU) per milliliter; bar graph represents average of three independent replicates, with individual data points overlaid. FIG. 5C shows growth curves in liquid culture for B. subtilis BEST7003 bacteria that contain exogenous DSR2 compared to control empty vector bacteria, infected by phage SPR at 30° C. Bacteria were infected at time 0 at an MOI of 0.04 or 4. Y axis represents bacterial density, as measured by optical density at a wavelength of 600 nm. Three independent replicates are shown for each MOI, and each curve shows an individual replicate. FIG. 5D-E demonstrate concentrations of NAD (FIG. 5D) and ADPR (FIG. 5E) in cell lysates extracted from SPR-infected cells as measured by targeted LC-MS with synthesized standards. X-axis represents minutes post infection, with zero representing non-infected cells. Cells were infected by phage SPR at an MOI of 4 at 30° C. Each panel shows data acquired for B. subtilis BEST7003 bacteria cloned with exogenous DSR2, the indicated mutants or control empty vector. Bar graphs represent the average of two biological replicates, with individual data points overlaid.

FIGS. 6A-D demonstrates that phage mating reveals phage that activate or repress DSR2. FIG. 6A shows the genes of SPR, SPBeta and Phi3T aligned based on amino acids sequence homology. Grey bands connect homologous genes. FIG. 6B is a schematic representation of the phage mating experiment. B. subtilis BEST7003 expressing both DSR2 and pVip is co-infected with phage SPR and SPbeta (or phi3T). Recombination between co-infecting phage genomes leads to hybrid phages that can overcome both defense systems, and generate plaques. Examination of the genomes of multiple hybrids predicts genomic regions necessary to overcome defense. FIG. 6C shows Plaque assays with single and double phages. Cells expressing both DSR2 and pVip are infected either with phage SPR (left), phage SPbeta (middle) or both phages (right). Each phage was added at a titer of 10⁷ pfu/ml. In FIG. 6D each horizontal line represents a hybrid phage that can overcome DSR2. Shades of gray differentiate between areas from SPR, SPbeta, or phi3T. Representative non-redundant hybrid sequences are presented out of 31 sequenced hybrids. Rectangles outline two areas that are predicted to allow the phage to overcome DSR2 defense. Top zoom inset shows genes found in the region acquired from phi3T or SPbeta. Bottom zoom inset shows genes present in the original SPR genome.

FIG. 7 demonstrates that the pVip gene protects bacteria against phages phi3T and SPbeta, but not from SPR. The efficiency of plating is shown for the three phages infecting the control B. subtilis BEST7003 strain which has an empty vector (“no system”) or a strain with cloned pVip. Data represent plaque-forming units (PFU) per milliliter; bar graph represents average of three independent replicates, with individual data points overlaid.

FIGS. 8A-G demonstrate that DSAD1 inhibits DSR2 defense system and phage tail tube protein activates DSR2. FIG. 8A shows growth curves in liquid culture for B. subtilis BEST7003 bacteria that express exogenous DSR2, bacteria that co-express DSR2 and DSAD1, and negative control bacteria cloned with an empty vector, infected by phage SPR at 30° C. Nucleic acid and amino acid sequences of DSR2 are provided in SEQ ID NOs: 746 and 753, nucleic acid and amino acid sequences of DSAD1 are provided in SEQ ID Nos: 738 and 739. Bacteria were infected at time 0 at an MOI of 0.04. Three independent replicates are shown for each MOI, and each curve shows an individual replicate. FIG. 8B shows pulldown assays of the DSR2-DSAD1 and DSR2-tail tube complex. DSAD1 and tail-tube proteins were tagged with streptavidin in the C-terminus. In this experiment, DSR2 was mutated (H171A) to avoid toxicity. Cell lysates were ran on SDS page gel after streptavidin affinity purification. Lane A, E. coli MG1655 cells expressing DSR2. Lane B, E. coli MG1655 cells expressing DSAD1 with tag. Lane C, E. coli MG1655 cells expressing tail-tube with tag. Lane D, E. coli MG1655 cells co-expressing DSR2 and DSAD1 with tag. Lane E, E. coli MG1655 cells co-expressing DSR2 and tail-tube with tag. FIG. 8C shows transformation efficiency measured for B. subtilis BEST7003 cells containing wild type or mutated DSR2, with a vector containing the tail-tube protein. As a control, a vector containing GFP was used to transform cells containing DSR2. Y-axis represents the number of colony forming units per milliliter. X-axis shows the different strains being transformed. FIG. 8D shows liquid culture growth of E. coli bacteria that contain DSR2 and the phage tail-tube protein, each under the control of an inducible promoter, and control E. coli that contain inducible GFP and DSR2 protein. Expression was induced at time 0 by adding 0.2% arabinose (for the tail-tube protein) and 1 mM IPTG (for DSR2). Three independent replicates are shown, and each curve shows an individual replicate. FIGS. 8E-F show concentrations of NAD (FIG. 8E) and ADPR (FIG. 8F) in cell cells lysates extracted from induced E. coli MG1655 cells as measured by targeted LC-MS with synthesized standards. X-axis represents minutes post induction, with zero representing non-induced cells. Expression was induced by 0.2% arabinose and 1 mM IPTG at 37° C. Each panel shows data acquired for cells expressing DSR2 and tail-tube protein or for control cells that express GFP and DSR2 protein. Bar graphs represent the average of two biological replicates, with individual data points overlaid. FIG. 8G is a model for the mechanism of action of DSR2. Phage infection is sensed by the recognition of phage tail-tube protein through direct binding to DSR2. This triggers the enzymatic activity of the SIR2 domain to deplete the cell of NAD+ and lead to abortive infection. The phage anti-DSR2 protein DSAD1 inhibits DSR2 by direct binding.

FIG. 9 shows genome comparison of eleven phages from the SpBeta group. Sequence similarity between genes is marked by grey shading.

FIGS. 10A-B show results of phage infection experiments with eight phages of the SBSphiJ family (FIG. 10A) and eleven phages of the SpBeta family (FIG. 10B). Data represent plaque-forming units per ml (PFU/ml) of phages infecting control cells (“no system”), and cells infecting strains expressing each one of the examined defense systems. Shown is the average of three replicates, with individual data points overlaid.

FIGS. 11A-B demonstrate that the TAD1 homologs inhibit Thoeris defense. FIG. 11A shows multiple sequence alignment of the 10 TAD1 homologs that were experimentally verified (nucleic acid sequences of the TAD1 homologs are provided in SEQ ID Nos: 321, 269, 281, 282, 85, 53, 49, 94, 188 and 174, amino acid sequences of the TAD1 homologs are provided in SEQ ID Nos: 324, 337, 338, 345, 400, 404, 438, 460, 464 and 543). Conserved residues are colored in grey. FIG. 11B shows results of phage infection experiments with eight phages of the SBSphiJ family. Data represent plaque-forming units per ml (PFU/ml) of phages infecting control cells (“no system”), cells expressing the Thoeris system (“Thoeris”), and cells expressing the Thoeris system and a TAD1 homolog. All phages except for SBSphiJ7 lack endogenous TAD1. Shown is the average of three replicates, with individual data points overlaid.

FIGS. 12A-D demonstrate that the HAD1/GAD1 homologs inhibit defense of the respective defense system. FIG. 12A shows multiple sequence alignment of the 5 HAD1 homologs that were chosen to be tested experimentally (nucleic acid sequences of the HAD1 homologs are provided in SEQ ID Nos: 581, 578, 588 and 593, amino acid sequences of the HAD1 homologs are provided in SEQ ID Nos: 598, 604, 607 and 609). Conserved residues are colored in grey. FIG. 12B shows results of phage infection experiments with eight phages of the SBSphiJ family. Data represent plaque-forming units per ml (PFU/ml) of phages infecting control cells (“no system”), cells expressing the Hachiman system (“Hachiman”), and cells expressing the Hachiman system and a HAD1 homolog. All phages except for SBSphiJ4 lack endogenous HAD1. Shown is the average of three replicates, with individual data points overlaid. FIG. 12C shows multiple sequence alignment of the 5 GAD1 homologs that were chosen to be tested experimentally (nucleic acid sequences of the GAD1 homologs are provided in SEQ ID Nos: 617, 645, 647 and 644, amino acid sequences of the GAD1 homologs are provided in SEQ ID Nos: 689, 723, 725 and 726). Conserved residues are colored in grey. FIG. 12D shows results of phage infection experiments with eleven phages of the SpBeta family. Data represent plaque-forming units per ml (PFU/ml) of phages infecting control cells (“no system”), cells expressing the Gabija system (“Gabija”), and cells expressing the Gabija system and a GAD1 homolog. Shown is the average of three replicates, with individual data points overlaid.

FIG. 13 demonstrates that TAD1 does not form a physical contact with ThsA or ThsB. Cell lysates were run on SDS page gel. Results show that His-tagged TAD1 protein does not pull down ThsA/ThsB. Left lane—cells expressing His-TAD1, center lane—cells expressing His-TAD1 and Thoeris (ThsA/B), right lane—cells expressing Thoeris (ThsA/B).

FIGS. 14A-B demonstrate that TAD2 inhibit Thoeris defense system. FIG. 14A demonstrates the differential defense of Thoeris against SPO1-like phages and the anti-Thoeris activity of TAD2. Data represent plaque-forming units per milliliter (PFU ml⁻¹) of phages infecting control cells (“no system”), cells expressing the Thoeris system (“Thoeris”) and cells co-expressing the Thoeris system and the TAD2 gene from SPO1 (“Thoeris+Tad2”). Nucleic acid and amino acid sequences of Thoeris are provided in SEQ ID NO: 741 and 1296-1297, nucleic acid and amino acid sequences of TAD2 are provided in SEQ ID Nos: 757 and 1046. All phages except for SPO1 and SPO1L3 lack an endogenous TAD2. Shown is the average of three replicates, with individual data points overlaid. FIG. 14B demonstrates that TAD2 knockdown cancels the anti-Thoeris activity, as determined by phage SPO1 infection experiments. Data represent plaque-forming units per ml (PFU/ml) of SPO1 infecting cells expressing Thoeris and a dCAS9 system targeting TAD2, as well as control cells. Shown is the average of three replicates, with individual data points overlaid.

FIG. 15 shows phylogenetic analysis of TAD2 homologs in phage and prophage genomes. The tree is based on 250 non-redundant sequences.

FIG. 16 demonstrates that TAD2 homologs inhibit the Thoeris system in B. subtilis. Results of phage infection experiments with six phages of the SPO1 family. Data represent PFU/mL of phages infecting control cells without Thoeris, cells expressing the Thoeris system, and cells co-expressing the Thoeris system and a TAD2 homolog. Nucleic acid and amino acid sequences of Thoeris are provided in SEQ ID NO: 741 and 1296-1297, nucleic acid and amino acid sequences of TAD2 homologs are provided in SEQ ID Nos: 758-761 and 1047-1050. All phages except for SPO1 and SPO1L3 lack endogenous TAD2. Shown is the average of three replicates, with individual data points overlaid.

FIG. 17 demonstrates that TAD2 releases bound molecule when denatured. Shown is NADase activity of purified ThsA incubated with mediums containing 900 nM of the Thoeris immune signaling molecule, that were additionally pre-incubated with purified TAD2 in vitro for 10 minutes, followed by an additional incubation of 5 minutes at either 25° C. or 95° C. “24 μM Tad2”—mediums pre-incubated with TAD2; “No Tad2”—mediums incubated with buffer instead of Tad2; “Control”—mediums not containing the Thoeris immune signaling molecule.

FIG. 18 demonstrates that TAD2 from a prophage integrated into Myroides odoratus XSfan inhibits the Thoeris defense system from Bacillus amyloliquefaciens Y2. Shown are tenfold serial dilution plaque assays with nine phages of the SBSphiJ family, infecting control B. subtilis cells without Thoeris, B. subtilis cells expressing the Thoeris system from Bacillus amyloliquefaciens Y2, and B. subtilis cells coexpressing the Thoeris system from Bacillus amyloliquefaciens Y2 and a Tad2 homolog from a prophage integrated into Myroides odoratus XSfan. Nucleic acid and amino acid sequences of Thoeris are provided in SEQ ID NO: 1300 and 1301-1302, nucleic acid and amino acid sequences of the TAD2 homolog are provided in SEQ ID Nos: 762 and 1051.

### DESCRIPTION OF SPECIFIC EMBODIMENTS OF THE INVENTION

The present invention, in some embodiments thereof, relates to genetically modified phages and used thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The evolutionary pressure imposed by phage predation on bacteria has resulted in the development of both anti-phage bacterial defense systems and counter-resistance mechanisms developed by phages that allow them to overcome bacterial defenses. Properly formulated and applied phages or their defense mechanisms have sufficient potential to cure bacterial infections.

Whilst conceiving embodiments of the invention, the present inventors have now uncovered that phages encode an arsenal of anti-defense proteins that can disable a wide variety of bacterial defense mechanisms and increase sensitivity of bacteria to phage infection.

Specifically, as is illustrated hereinunder and in the examples section which follows, by comparing genomically-similar Bacillus phages that showed differential sensitivity to bacterial defense systems, the present inventors identified five families of anti-defense proteins that inhibit the previously described Thoeris, Hachiman, Gabija and DSR2 bacterial defense systems (Examples 1-6). Homologs of these anti-defense proteins were found in hundreds of phages that infect taxonomically diverse bacterial species, and by cloning such homologs into Bacillus subtilis cells that express Thoeris, Hachiman, Gabija or DSR2 bacterial defense system, the present inventors show that they efficiently cancel the respective defensive activity (Examples 2-6). Additionally or alternatively, knocking out the anti-defense protein or homolog from the phage genome abrogated the phage ability to overcome the bacterial defense system (Examples 4 and 6).

Consequently, specific embodiments of the present teachings suggest genetically modified phages comprising a polynucleotide encoding these anti-defense proteins for use in combatting bacterial infections/contaminations.

Thus, according to an aspect of the present invention, there is provided a genetically modified phage comprising a polynucleotide encoding an anti-defense system polypeptide.

As used herein, the term “phage” or “bacteriophage” refers to a virus that selectively infects one or more bacterial species. Many phages are specific to a particular genus or species or strain of bacteria. According to specific embodiments, the phage genome can be ssDNA or dsDNA. According to specific embodiments, the phage genome can be ssRNA or dsRNA.

Typically, a phage will be characterized by: 1) the nature of the nucleic acids that make up its genome, e.g., DNA, RNA, single-stranded or double-stranded; 2) the nature of its infectivity, e.g., lytic or temperate; and 3) the particular bacterial species that it infects (and in certain instances the particular subspecies or strain). This is known as “host range”.

According to some embodiments, the phage is a lytic phage.

The term “lytic phage” refers to a phage that infects a bacterial host and causes that host to lyse without incorporating the phage nucleic acids into the host genome. A lytic phage is typically not capable of reproducing using the lysogenic cycle.

According to other embodiments, the phage is temperate (also referred to as lysogenic).

The term “temperate phage” refers to a phage that is capable of reproducing using both the lysogenic cycle and the lytic cycle. Lysogeny is characterized by integration of the bacteriophage nucleic acid into the host bacterium's genome or formation of a circular replicon in the bacterial cytoplasm.

According to specific embodiments, phages that infect bacteria that are pathogenic to plants and/or animals (including humans) find particular use.

Exemplary phages which fall under the scope of some embodiments of the invention include, but are not limited to, phages that belong to any of the following virus families: Corticoviridae, Cystoviridae, Inoviridae, Leviviridae, Microviridae, Myoviridae, Podoviridae, Siphoviridae, or Tectiviridae.

According to specific embodiments, the phage is selected from the phages listed in Tables 5-10 hereinbelow.]

According to specific embodiments, the phage is isolated and/or developed to target a specific bacteria. Isolation and characterization of such phages can be done, for example, as described in Hayman Pharmaceuticals (Basel) (2019) 12(1): 35, DOI: 10.3390/ph12010035 and www(dot)doi.org/10.1016/j.cels.2015.08.013, the contents of which are fully incorporated herein by reference.

According to specific embodiments, the phage infects a pathogenic bacteria (i.e. a bacteria that can cause or be associated with a disease in humans, livestock, crops, or other living organism).

According to specific embodiments, the phage is a clinically approved phage.

According to specific embodiments, the phage is FDA approved for treatment of an infectious bacterium.

The phage of some embodiments of the invention is genetically modified.

Hence, the phages of some embodiments of the invention comprise a heterologous nucleic acid sequence or residue.

The term “heterologous” as used herein, refers to a nucleic acid sequence or residue which is not native to the phage at least in localization or is completely absent from the native genomic sequence of the phage.

Methods of genetically modifying a phage are well known in the art and described in e.g. Gibb et al. (2021) Pharmaceuticals, 14, 634; Chen et al. (2019) Front, Microbiol, 10:954; and Rustad, et al. (2018) Synthetic Biology, 3(1) ysy002, doidotorg/10.1093/synbio/ysy002, the contents of which are fully incorporated herein by reference, and are further described hereinbelow and in the Examples section which follows. Non-limiting Examples include, homologous recombination, bacteriophage recombineering of electroporated DNA (BRED), CRISPRZ-Cas-based phage engineering and rebooting phages using assembled phage genomic DNA.

One Exemplary method of obtaining the genetically modified phage is by recombination-mediated genetic exchange between at least 2 distinct phages, as further described in the Examples section which follows which serve as an integral part of the specification of the instant application.

Thus, according to specific embodiments, the phage comprises genomic segments of a distinct phage integrated in a genome of said phage.

According to specific embodiments, these segments are about 1000-60,000, 2000-50,000 or 10,000-30, 000 long.

According to specific embodiments, these segments constitute about 0.5-50% of the phage genome.

According to specific embodiments, these segments constitute less than 40%, less than 30%, less than 20%, less than 10% of the phage genome.

According to specific embodiments, these segments constitute more than 1%, more than 5%, more than 10%, more than 20% of the phage genome.

According to specific embodiments, these segments constitute less than 1% of the phage genome.

The phage may be genetically engineered to change its host range, convert a temperate phage to a lytic phage, encode an anti-defense system polypeptide and the like.

According to specific embodiments, the genetically modified phage has an increased infectivity to at least one bacteria as compared to a non-genetically modified phage of the same species or strain.

As used herein, “increasing sensitivity” or “increased infectivity” refers to a significant increase in bacterial susceptibility towards a genetically modified phage, as compared to a non-genetically modified phage of the same species or strain, as may be manifested e.g. in growth arrest, death, integration of the phage nucleic acid sequence into the bacterial genome, prevention of lysogeny and/or phage genomic replication. According to a specific embodiment, the increase is in at least 5%, at least 10%, 20%, 30%, 40% or even higher say, 50%, 60%, 70%, 80%, 90% or more than 100%. According to specific embodiments the increase is at least 1.5 fold, at least 2 fold, at least 3 fold, at least 5 fold, at least 10 fold, or at least 20 fold.

Assays for testing phage sensitivity or infectivity are well known in the art, and are also described in the Examples section which follows. Thus, for example, the lysogenic activity of a phage can be assessed by PCR or DNA sequencing. The DNA replication activity of a phage can be assessed e.g. by DNA sequencing or southern blot analysis.

The lytic activity of a phage can be assessed e.g. by optical density, plaque assay or living dye indicators.

The lytic activity of a phage can be measured indirectly by following the decrease in optical density of the bacterial cultures owing to lysis. This method involves introduction of phage into a fluid bacterial culture medium. After a period of incubation, the phage lyses the bacteria in the broth culture resulting in a clearing of the fluid medium resulting in decrease in optical density.

Another method, known as the plaque assay, introduces phage into a few milliliters of soft agar along with some bacterial host cells. This soft agar mixture is laid over a hard agar base (seeded-agar overlay). The phage adsorbs onto the host bacterial cells, infect and lyse the cells, and then begin the process anew with other bacterial cells in the vicinity. After 6-24 hours, zones of clearing on the plate, known as plaques, are observable within the lawn of bacterial growth on the plate. Each plaque represents a single infective phage particle in the original sample.

Yet another method is the one-step phage growth curve which allows determining the production of progeny virions by cells as a function of time after infection. The assay is based on the fact that cells in the culture are infected simultaneously with a low number of phages so that no cell can be infected with more than one phage. At various time intervals, samples are removed for a plaque assay allowing quantitative determination of the number of phages present in the medium.

Other methods use for example redox chemistry, employing cell respiration as a universal reporter. During active growth of bacteria, cellular respiration reduces a dye (e.g., tetrazolium dye) and produces a color change that can be measured in an automated fashion. On the other hand, successful phage infection and subsequent growth of the phage in its host bacterium results in reduced bacterial growth and respiration and a concomitant reduction in color.

Non-limiting Examples of modifications that can be introduced to phage include genetic engineering of host-range-determining regions (HRDRs) in the tail fiber protein [see e.g. Yehl et al. Cell (2019) 179(2):459-469.e9 doi: 10.1016/j.cell.2019.09.015, the contents of which are fully incorporated herein by reference] or receptor-binding proteins [see e.g. Lenneman et al. Curr Opin Biotechnol (2021) 68:151-159 doi: 10.1016/j.copbio.2020.11.003, the contents of which are fully incorporated herein by reference].

The phages disclosed herein comprise a polynucleotide encoding an anti-defense system polypeptide.

According to specific embodiments, the polynucleotide encoding the anti-defense polypeptide is endogenous to the phage.

The term “endogenous” as used herein, refers to the expression of the native gene in its natural location and expression level in the genome of a phage.

According to other specific embodiments, the phage is devoid of an endogenous polynucleotide encoding the anti-defense polypeptide.

Thus, according to specific embodiments, the polynucleotide encoding the anti-defense polypeptide is heterologous to the phage.

As used herein, “anti-defense system polypeptide” refers to a polypeptide which expression in an infected bacteria disables an anti-phage bacterial defense system, thereby increasing sensitivity of the bacteria to a phage.

Numerous anti-phage bacterial defense systems are known in the art, including, but not limited to CRISPR-Cas, a restriction modification, toxin anti-toxin, BREX, DISARM, Thoeris, Hachiman, Gabija, DSR2, Septu and Lamassu systems (see e.g. Labrie et al Nature Reviews Microbiology 8, 317-327 (2010); Bernheim, A. & Sorek, R. Nat. Rev. Microbiol. 18, 113-119 (2020); Goldfarb et al. EMBO J. (2015) 34, 169-83; Doron, S. et al. Science 359, eaar4120 (2018); and International Application Publication Nos. WO2015/059690, WO2018142416 and WO2018/220616, the contents of which are fully incorporated herein by reference).

According to specific embodiments, the anti-phage bacterial defense system is not CRISPR-Cas, not a restriction modification system and/or not a toxin anti-toxin system.

According to specific embodiments, the anti-phage bacterial defense system is not DISARM.

According to specific embodiments, the anti-phage bacterial defense system is selected from the group consisting of Thoeris, Hachiman, Gabija and DSR2, as further described hereinbelow.

According to specific embodiments, the anti-defense system polypeptides is a TAD1 polypeptide. A TAD1 polypeptide disables the anti-phage bacterial defense system Thoeris described in Doron, S. et al. Science 359, eaar4120 (2018); and International Application Publication No. WO2018/220616. Hence, expression of a TAD1 polypeptide in a B. subtilis BEST7003 comprising the Thoeris system as provided in SEQ ID NO: 741 increases sensitivity of the B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6, such as determined by a plaque assay or liquid culture infection e.g. as described in the Examples section which follows.

According to specific embodiments, the TAD1 polypeptide inhibits Thoeris defense by binding and chelating the TIR-derived signaling molecule, thus disallowing Thoeris effector activation and preventing the Thoeris-mediated premature cell death, such as determined by measuring the ability of filtered cell lysates derived from Thoeris-infected cells to activate the Thoeris effector ThsA (e.g. the NADase activity of the Thoeris effector ThsA).

The TAD1 polypeptide of some embodiments has at least 80% identity to an amino acid sequence selected from the group consisting of SEQ ID NOs: 22 and 323-577, each possibility represents a separate embodiment of the claimed invention.

According to specific embodiments, the TAD1 polypeptide has at least 80% identity to an amino acid sequence selected from the group consisting of SEQ ID NOs: 22, 324, 337, 338, 345, 400, 404, 438, 460, 464 and 543.

The TAD1 polypeptide of some embodiments has an e-value≤0.05 to an amino acid sequence selected from the group consisting of SEQ ID NO: 22 and 323-577, each possibility represents a separate embodiment of the claimed invention.

According to specific embodiments, the TAD1 polypeptide has an e-value≤0.05 to an amino acid sequence selected from the group consisting of SEQ ID NOs: 22, 324, 337, 338, 345, 400, 404, 438, 460, 464 and 543.

According to specific embodiments, the anti-defense system polypeptides is a HAD1 polypeptide. A HAD1 polypeptide disables the anti-phage bacterial defense system Hachiman described in Doron, S. et al. Science 359, eaar4120 (2018); and International Application Publication No. WO2018/220616. Hence, expression of a HAD1 polypeptide in a B. subtilis BEST7003 comprising the Hachiman system as provided in SEQ ID NO: 742 increases sensitivity of the B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7, such as determined by a plaque assay e.g. as described in the Examples section which follows.

The HAD1 polypeptide of some embodiments has at least 80% identity to an amino acid sequence selected from the group consisting of SEQ ID NOs: 32 and 595-610, each possibility represents a separate embodiment of the claimed invention.

According to specific embodiments, the HAD1 polypeptide has at least 80% identity to an amino acid sequence selected from the group consisting of SEQ ID NOs: 32, 598, 604, 607 and 609.

The HAD1 polypeptide of some embodiments has an e-value≤0.05 to an amino acid sequence selected from the group consisting of SEQ ID NOs: 32 and 595-610, each possibility represents a separate embodiment of the claimed invention.

According to specific embodiments, the HAD1 polypeptide has an e-value≤0.05 to an amino acid sequence selected from the group consisting of SEQ ID NOs: 32, 598, 604, 607 and 609.

According to specific embodiments, the anti-defense system polypeptides is a GAD1 polypeptide. A GAD1 polypeptide disables the anti-phage bacterial defense system Gabija described in Doron, S. et al. Science 359, eaar4120 (2018); and International Application Publication No. WO2018/220616. Hence, expression of a GAD1 polypeptide in a B. subtilis BEST7003 comprising the Gabija system as provided in SEQ ID NO: 740 increases sensitivity of the B. subtilis BEST7003 to infection by at least one B. subtilis phage selected from the group consisting of SpBeta, SpBetaL6, SpBetaL7 and SpBetaL8, such as determined by a plaque assay e.g. as described in the Examples section which follows.

The GAD1 polypeptide of some embodiments has at least 80% identity to an amino acid sequence selected from the group consisting of SEQ ID NOs: 33 and 675-737, each possibility represents a separate embodiment of the claimed invention.

According to specific embodiments, the GAD1 polypeptide has at least 80% identity to an amino acid sequence selected from the group consisting of SEQ ID NOs: 33, 689, 723, 725 and 726.

The GAD1 polypeptide of some embodiments has e-value≤0.05 to an amino acid sequence selected from the group consisting of SEQ ID NOs: 33 and 675-737, each possibility represents a separate embodiment of the claimed invention.

According to specific embodiments, the GAD1 polypeptide embodiments has an e-value≤0.05 to an amino acid sequence selected from the group consisting of SEQ ID NOs: 33, 689, 723, 725 and 726.

According to specific embodiments, the anti-defense system polypeptides is a DSAD1 polypeptide. A DSAD1 polypeptide disables the anti-phage bacterial defense system DSR2, described in Gao et al. Science (2020) 369(6507):1077-1084, doi: 10.1126/science.aba0372. Hence, expression of a DSAD1 polypeptide in a B. subtilis BEST7003 comprising the DSR2 system as provided in SEQ ID NO: 746 increases sensitivity of the B. subtilis BEST7003 to infection by at a B. subtilis phage SPR, such as determined by a plaque assay or a liquid infection growth curves, e.g. as described in the Examples section which follows.

According to specific embodiments, the DSAD1 polypeptide inhibits DSR2 defense by binding a DSR2 polypeptide (e.g. SEQ ID NO: 753) and inhibiting its NADase activity in response to phage infection, such as determined by measuring the binding of the polypeptide to DSR2, e.g. as described in the Examples section which follows, and measuring the NAD+ concentrations in infected cells that contain DSR2 when DSAD1, e.g. as described in the Examples section which follows.

The DSAD1 polypeptide of some embodiments has an e-value≤0.05 to SEQ ID NO: 739.

The DSAD1 polypeptide of some embodiments has at least 80% identity to SEQ ID NO: 739.

The DSAD1 polypeptide of some embodiments has at least 35% identity and an e-value≤0.05 to SEQ ID NO: 739.

According to specific embodiments, the anti-defense system polypeptides is a TAD2 polypeptide. A TAD2 polypeptide disables one or more representations of the anti-phage bacterial defense system Thoeris described in Doron, S. et al. Science 359, eaar4120 (2018); and International Application Publication No. WO2018/220616. Hence, expression of a TAD2 polypeptide in a B. subtilis BEST7003 comprising the Thoeris system as provided in SEQ ID NO: 741 or 1300 increases sensitivity of the B. subtilis BEST7003 to infection by at least one B. subtilis phage selected from the group consisting of SPO1L1, SPO1L2, SPO1L4 and SPO1L5, such as determined by a plaque assay or liquid culture infection e.g. as described in the Examples section which follows.

According to specific embodiments, the TAD2 polypeptide inhibits Thoeris defense by binding and chelating the TIR-derived signaling molecule, thus disallowing Thoeris effector activation and preventing the Thoeris-mediated premature cell death, such as determined by measuring the ability of filtered cell lysates derived from Thoeris-infected cells to activate the Thoeris effector ThsA.

The TAD2 polypeptide of some embodiments has at least 80% identity to an amino acid sequence selected from the group consisting of SEQ ID Nos: 1046-1295, each possibility represents a separate embodiment of the claimed invention.

According to specific embodiments, the TAD2 polypeptide has at least 80% identity to an amino acid sequence selected from the group consisting of SEQ ID NOs: 1046-1051.

The TAD2 polypeptide of some embodiments has an e-value≤0.05 to an amino acid sequence selected from the group consisting of SEQ ID NO: 1046-1295, each possibility represents a separate embodiment of the claimed invention.

According to specific embodiments, the TAD2 polypeptide has an e-value≤0.05 to an amino acid sequence selected from the group consisting of SEQ ID NOs: 1046-1051.

As used herein, “percent identity”, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Sequence identity (or homology) can be determined using any protein or nucleic acid sequence alignment algorithm such as Blast, ClustalW, MUSCLE, Mmseqs2, and HHpred.

In some embodiments, the sequence alignment program is a basic local alignment program, e.g., BLAST.

According to some embodiments of the invention, the identity is a global identity, i.e., an identity over the entire sequences of the invention and not over portions thereof.

According to specific embodiments, the polypeptide has at least 35% identity to the recited SEQ ID NO.

According to specific embodiments, the at least 35% identity comprises at least 40%, at least 45%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% or 100% identity.

According to specific embodiments, the polypeptide has at least 80% identity to the recited SEQ ID NO.

According to specific embodiments, the at least 80% identity comprises at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% or 100% identity.

According to specific embodiments, the at least 80% identity comprises at least 90% identity.

According to specific embodiments, the at least 80% identity comprises at least 95% identity.

As used herein, “e-value” refers to a parameter that describes the number of hits one can expect to see by chance in a sequence alignment (or homology) search to the recited sequence using a database of a particular size. It decreases exponentially as the Score (S) of the match increases. Essentially, the E value describes the random background noise. An E value of I assigned to a hit can be interpreted as meaning that in a database of the current size one might expect to see I match with a similar score simply by chance.

According to specific embodiments, the e-value represents a statistically significant homology or identity to the recited sequence.

Thus, according to specific embodiments, the e-value is ≤0.05.

According to specific embodiments, the e-value is ≤0.01, ≤0.001 or ≤0.0001.

Thus, the “TAD1 polypeptide”, “TAD2 polypeptide”, “HAD1 polypeptide”, “GAD1 polypeptide” and/or “DSAD1 polypeptide” may comprise a homolog, an ortholog, a deletion, insertion, or substitution variant, including an amino acid substitution of the recited amino acid sequence.

According to a specific embodiment, the “TAD1 polypeptide”, “TAD2 polypeptide” “HAD1 polypeptide”, “GAD1 polypeptide” and/or “DSAD1 polypeptide” comprises the recited amino acid sequence.

According to a specific embodiment, the “TAD1 polypeptide”, “TAD2 polypeptide”, “HAD1 polypeptide”, “GAD1 polypeptide” and/or “DSAD1 polypeptide” consists of the recited amino acid sequence.

According to specific embodiments, the terms “TAD1 polypeptide”, “TAD2 polypeptide”, “HAD1 polypeptide”, “GAD1 polypeptide” and/or “DSAD1 polypeptide” refer to a fragment of the amino acid sequence of “TAD1 polypeptide”, “TAD2 polypeptide”, “HAD1 polypeptide”, “GAD1 polypeptide” and/or “DSAD1 polypeptide” respectively, provided herein, which maintains the activity as described herein.

According to specific embodiments, the TAD1 polypeptide is 100-250 amino acids long, or 100-200 amino acids long.

According to specific embodiments, the HAD1 polypeptide is 40-150 amino acids long or 40-100 amino acids long.

According to specific embodiments, the GAD1 polypeptide is 100-400 amino acids long or 200-350 amino acids long.

According to specific embodiments, the TAD2 polypeptide is 30-300 amino acids long, 40-280 amino acids long, 80-280 amino acids long or 80-200 amino acids long.

According to specific embodiments, the TAD2 polypeptide is at least 80 amino acids long.

According to specific embodiments, the phage comprises a polynucleotide encoding one of the anti-defense polypeptides (i)-(v) described herein.

According to specific embodiments, the phage comprises a polynucleotide encoding one of the anti-defense polypeptides (i)-(iv) described herein.

According to specific embodiments, the phage comprises a polynucleotide encoding at least two, at least three, at least four or five of the anti-defense polypeptides (i)-(v) described herein.

Hence, according to specific embodiments, the phage comprises a polynucleotide encoding (i)+(ii), (i)+(iii), (i)+(iv), (i)+(v), (ii)+(iii), (ii)+(iv), (ii)+(v), (iii)+(iv), (iii)+(v), (i)+(ii)+(iii), (i)+(ii)+(iv), (i)+(ii)+(v), (i)+(iii)+(iv), (i)+(iii)+(v), (ii)+(iii)+(iv), (ii)+(iii)+(v), (ii)+(iv)+(v), (iii)+(iv)+(v), (i)+(ii)+(iii)+(iv), (i)+(ii)+(iii)+(v), (i)+(ii)+(iv)+(v), (i)+(iii)+(iv)+(v), (ii)+(iii)+(iv)+(v) or (i)+(ii)+(iii)+(iv)+(v), each possibility represents a separate embodiment of the present invention.

According to specific embodiments, the phage comprises a polynucleotide encoding at least two, at least three or four of the anti-defense polypeptides (i)-(iv) described herein.

Hence, according to specific embodiments, the phage comprises a polynucleotide encoding (i)+(ii), (i)+(iii), (i)+(iv), (ii)+(iii), (ii)+(iv), (iii)+(iv), (i)+(ii)+(iii), (i)+(ii)+(iv), (i)+(iii)+(iv) or (i)+(ii)+(iii)+(iv), each possibility represents a separate embodiment of the present invention.

Non-limiting examples of polynucleotides encoding TAD1 are provided in SEQ ID NOs: 5, 35-322.

Non-limiting examples of polynucleotides encoding HAD1 are provided in SEQ ID NOs: 15, 578-594.

Non-limiting examples of polynucleotides encoding GAD1 are provided in SEQ ID NOs: 16, 611-674.

Non-limiting examples of polynucleotides encoding DSAD1 are provided in SEQ ID NO: 738.

Non-limiting examples of polynucleotides encoding TAD2 are provided in SEQ ID NOs: 757-1045.

According to specific embodiments, the polynucleotide encoding the “TAD1 polypeptide”, “TAD2 polypeptide”, “HAD1 polypeptide”, “GAD1 polypeptide” or “DSAD1 polypeptide” is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the SEQ ID NOs: 5, 35-322; 757-1045; 15, 578-594; 16, 611-674; or 738, respectively.

Various modalities may be used to introduce or express a heterologous polynucleotide encoding the anti-defense polypeptide in the phage, as further described hereinabove and below.

Thus, according to an aspect of the present invention there is provided a method of producing a phage, the method comprising contacting the phage with a polynucleotide encoding the anti-defense polypeptide, under conditions which allow integration of said polynucleotide in a genome of said phage, thereby producing the phage.

Various methods known within the art can be used to contacting the phage with the polynucleotide. Such methods are described for example in Gibb et al. (2021) Pharmaceuticals, 14, 634; Chen et al. (2019) Front. Microbiol. 10:954; and Rustad, et al. (2018) Synthetic Biology, 3(1) ysy002, doidotorg/10.1093/synbio/ysy002, the contents of which are fully incorporated herein by reference.

As used herein the term “polynucleotide” or “nucleic acid sequence”, which are interchangeably used herein, refers to a single or double stranded nucleic acid sequence provided e.g., in the form of an RNA sequence, a DNA and/or a composite polynucleotide sequences (e.g., a combination of the above).

According to specific embodiments, the polynucleotide of the present invention encodes no more than 20, no more than 15, no more than 10 genes expression products.

According to specific embodiments, the polynucleotide encodes one of the anti-defense polypeptides (i)-(v) described herein.

According to specific embodiments, the polynucleotide encodes one of the anti-defense polypeptides (i)-(iv) described herein.

According to specific embodiments, the polynucleotide comprises a nucleic acid sequence encoding one of the anti-defense polypeptides (i)-(v) described herein, whereby a plurality of polynucleotides can be used to assemble several anti-defense polypeptides, as described below.

According to specific embodiments, the polynucleotide comprises a nucleic acid sequence encoding one of the anti-defense polypeptides (i)-(iv) described herein, whereby a plurality of polynucleotides can be used to assemble several anti-defense polypeptides, as described below.

According to other specific embodiments, a single polynucleotide encodes at least two, at least three, at least four or five of the anti-defense polypeptides (i)-(v) described herein. Further description on expression of multiple polypeptides from a single polynucleotide is provided hereinbelow.

Hence, according to specific embodiments, the polynucleotide encodes (i)+(ii), (i)+(iii), (i)+(iv), (i)+(v), (ii)+(iii), (ii)+(iv), (ii)+(v), (iii)+(iv), (iii)+(v), (i)+(ii)+(iii), (i)+(ii)+(iv), (i)+(ii)+(v), (i)+(iii)+(iv), (i)+(iii)+(v), (ii)+(iii)+(iv), (ii)+(iii)+(v), (ii)+(iv)+(v), (iii)+(iv)+(v), (i)+(ii)+(iii)+(iv), (i)+(ii)+(iii)+(v), (i)+(ii)+(iv)+(v), (i)+(iii)+(iv)+(v), (ii)+(iii)+(iv)+(v) or (i)+(ii)+(iii)+(iv)+(v), each possibility represents a separate embodiment of the present invention.

According to other specific embodiments, a single polynucleotide encodes at least two, at least three or four of the anti-defense polypeptides (i)-(iv) described herein. Further description on expression of multiple polypeptides from a single polynucleotide is provided hereinbelow.

Hence, according to specific embodiments, the polynucleotide encodes (i)+(ii), (i)+(iii), (i)+(iv), (ii)+(iii), (ii)+(iv), (iii)+(iv), (i)+(ii)+(iii), (i)+(ii)+(iv), (i)+(iii)+(iv) or (i)+(ii)+(iii)+(iv), each possibility represents a separate embodiment of the present invention.

According to specific embodiments, in order to allow expression of the polypeptides described herein in an infected bacteria, the polynucleotides described herein are part of a nucleic acid construct (also referred to herein as an “expression vector” or a “vector”) which facilitates expression and/or integration of the polynucleotide in a phage genome.

Hence, according to an aspect of the present invention, there is provided a nucleic acid construct comprising a polynucleotide encoding an anti-defense polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of said TAD1 polypeptide in a B. subtilis BEST7003 comprising SEQ ID NO: 741 increases sensitivity of said B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of said HAD1 polypeptide in a B. subtilis BEST7003 comprising SEQ ID NO: 742 increases sensitivity of said B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of said GAD1 polypeptide in a B. subtilis BEST7003 comprising SEQ ID NO: 740 increases sensitivity of said B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of said DSAD1 polypeptide in a B. subtilis BEST7003 comprising SEQ ID NO: 746 increases sensitivity of said B. subtilis BEST7003 to infection by a B. subtilis phage SPR; and
(v) a TAD2 polypeptide having at least 80% identity and/or an e-value≤0.05 to an amino acid sequence selected from the group consisting of SEQ ID NO: 1046-1295, and wherein expression of said TAD2 polypeptide in a B. subtilis BEST7003 comprising SEQ ID NO: 741 or 1300 increases sensitivity of said B. subtilis BEST7003 to infection by at least one B. subtilis phage selected from the group consisting of SPO1L1, SPO1L2, SPO1L4 and SPO1L5; and a nucleic acid sequence heterologous to said polynucleotide which facilitates expression and/or integration of said polynucleotide in a phage genome.

According to an additional or an alternative aspect of the present invention, there is provided a nucleic acid construct comprising a polynucleotide encoding an anti-defense polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of said TAD1 polypeptide in a B. subtilis BEST7003 comprising SEQ ID NO: 741 increases sensitivity of said B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of said HAD1 polypeptide in a B. subtilis BEST7003 comprising SEQ ID NO: 742 increases sensitivity of said B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of said GAD1 polypeptide in a B. subtilis BEST7003 comprising SEQ ID NO: 740 increases sensitivity of said B. subtilis BEST7003 to infection by at least one B. subtilis phage selected from the group consisting of SpBeta, SpBetaL6, SpBetaL7, SpBetaL8; and
(iv) a DSAD1 polypeptide having at least 80% identity and/or an e-value≤0.05 to SEQ ID NO: 739, and wherein expression of said DSAD1 polypeptide in a B. subtilis BEST7003 comprising SEQ ID NO: 746 increases sensitivity of said B. subtilis BEST7003 to infection by a B. subtilis phage SPR;
   and a nucleic acid sequence heterologous to said polynucleotide which facilitates expression and/or integration of said polynucleotide in a phage genome.

Such a nucleic acid construct includes at least one cis-acting regulatory element for directing expression of the nucleic acid sequence.

According to specific embodiments, the nucleic acid sequence heterologous to the polynucleotide encoding the anti-defense polypeptide is a cis-acting regulatory element for directing expression of the polynucleotide in an infected bacteria.

Cis-acting regulatory sequences include those that direct constitutive expression of a nucleotide sequence as well as those that direct inducible expression of the polynucleotide only under certain conditions. Thus, for example, a promoter sequence for directing transcription of the polynucleotide sequence in the bacteria in a constitutive or inducible manner is included in the nucleic acid construct.

Non-limiting examples of constitutive promoters suitable for use with some embodiments of the invention include T7, Sp6 and T3. Non-limiting Examples of inducible promoters suitable for use with some embodiments of the invention include the tetracycline-inducible promoter (Zabala M, et al., Cancer Res. 2004, 64(8): 2799-804), the arabinose metabolic operon promoter (araBAD) or pathogen-inducible promoters. Such promoters include those from pathogenesis-related proteins (PR proteins), which are induced following infection by a pathogen.

According to specific embodiments the promoter is a bacterial promoter.

A bacterial promoter is any DNA sequence capable of binding bacterial RNA polymerase and initiating the downstream (3′) transcription of a coding sequence into mRNA. A promoter can have a transcription initiation region, which is usually placed proximal to the 5′ end of the coding sequence. This transcription initiation region typically includes an RNA polymerase binding site and a transcription initiation site. A bacterial promoter can also have a second domain called an operator, which can overlap an adjacent RNA polymerase binding site at which RNA synthesis begins. The operator permits negative regulated (inducible) transcription, as a gene repressor protein can bind the operator and thereby inhibit transcription of a specific gene. Constitutive expression can occur in the absence of negative regulatory elements, such as the operator. In addition, positive regulation can be achieved by a gene activator protein binding sequence, which, if present is usually proximal (5′) to the RNA polymerase binding sequence.

An example of a gene activator protein is the catabolite activator protein (CAP), which helps initiate transcription of the lac operon in Escherichia coli (Raibaud et al. (1984) Annu. Rev. Genet. 18:173). Regulated expression can therefore be either positive or negative, thereby either enhancing or reducing transcription. Other examples of positive and negative regulatory elements are well known in the art. Various promoters that can be included in the protein expression system include, but are not limited to, a T7/LacO hybrid promoter, a trp promoter, a T7 promoter, a lac promoter, and a bacteriophage lambda promoter.

Sequences encoding metabolic pathway enzymes provide particularly useful promoter sequences. Examples include promoter sequences derived from sugar metabolizing enzymes, such as galactose, lactose (lac) (Chang et al. (1987) Nature 198:1056), and maltose. Additional examples include promoter sequences derived from biosynthetic enzymes such as tryptophan (trp) (Goeddel et al. (1980) Nucleic Acids Res. 8:4057; Yelverton et al. (1981) Nucleic Acids Res. 9:731; U.S. Pat. No. 4,738,921; EPO Publication Nos. 36,776 and 121,775). The beta-lactamase (bla) promoter system (Weissmann, (1981) “The Cloning of Interferon and Other Mistakes,” in Interferon 3 (ed. I. Gresser); bacteriophage lambda PL (Shimatake et al. (1981) Nature 292:128); the arabinose-inducible araB promoter (U.S. Pat. No. 5,028,530); and T5 (U.S. Pat. No. 4,689,406) promoter systems also provide useful promoter sequences. See also Balbas (2001) Mol. Biotech. 19:251-267, where E. coli expression systems are discussed.

In addition, synthetic promoters that do not occur in nature also function as bacterial promoters. For example, transcription activation sequences of one bacterial or phage promoter can be joined with the operon sequences of another bacterial or phage promoter, creating a synthetic hybrid promoter (U.S. Pat. No. 4,551,433). For example, the tac (Amann et al. (1983) Gene 25:167; de Boer et al. (1983) Proc. Natl. Acad. Sci. 80:21) and trc (Brosius et al. (1985) J. Biol. Chem. 260:3539-3541) promoters are hybrid trp-lac promoters comprised of both trp promoter and lac operon sequences that are regulated by the lac repressor. The tac promoter has the additional feature of being an inducible regulatory sequence. Thus, for example, expression of a coding sequence operably linked to the tac promoter can be induced in a cell culture by adding isopropyl-1-thio-.beta.-D-galactoside (IPTG). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The phage T7 RNA polymerase/promoter system is an example of a coupled promoter system (Studier et al. (1986) J. Mol. Biol. 189:113; Tabor et al. (1985) Proc. Natl. Acad. Sci. 82:1074). In addition, a hybrid promoter can also be comprised of a phage promoter and an E. coli operator region (EPO Publication No. 267,851).

In the construction of the construct, the promoter is preferably positioned approximately the same distance from the heterologous transcription start site as it is from the transcription start site in its natural setting. As is known in the art, however, some variation in this distance can be accommodated without loss of promoter function.

Alternatively or additionally, the nucleic acid construct is designed to allow integration of the polynucleotide in a location enabling expression of the polynucleotide from a native phage promoter.

Hence, according to specific embodiments, the nucleic acid construct is devoid of a promoter.

In order to avoid negatively affecting phage infectivity and specificity, the polynucleotide sequence is typically not inserted inside an existing phage open reading frame.

The nucleic acid construct can additionally contain a nucleic acid sequence encoding the repressor (or inducer) for the promoter. For example, an inducible construct of the present invention can regulate transcription from the Lac operator (LacO) by expressing the nucleotide sequence encoding the LacI repressor protein. Other examples include the use of the lexA gene to regulate expression of pRecA, and the use of trpO to regulate ptrp. Alleles of such genes that increase the extent of repression (e.g., laclq) or that modify the manner of induction (e.g., lambda CI857, rendering lambda pL thermo-inducible, or lambda CI+, rendering lambda pL chemo-inducible) can be employed.

Various construct schemes can be utilized to express few genes from a single nucleic acid construct. According to specific embodiments, the construct has an operon structure. Alternatively, each two nucleic acid sequence segments can be translationally fused via a protease recognition site cleavable by a protease expressed by the cell to be transformed with the nucleic acid construct. In this case, a chimeric polypeptide translated will be cleaved by the cell expressed protease. Still alternatively, the nucleic acid construct of some embodiments of the invention can include at least two Cis acting regulatory elements (e.g. promoter) each being for separately expressing a distinct polynucleotide. These at least two Cis acting regulatory elements can be identical or distinct.

Hence, according to specific embodiments, the nucleic acid sequence heterologous to the polynucleotide encoding the anti-defense system polypeptide is an element for expression of multiple polynucleotides from a single construct.

The nucleic acid construct of some embodiments of the invention includes additional sequences which render this construct suitable for replication and integration in prokaryotes, eukaryotes, or preferably both (e.g., shuttle vectors).

According to specific embodiments, the nucleic acid sequence heterologous to the polynucleotide encoding the anti-defense system polypeptide is a transmissible genetic element.

In other words, according to specific embodiments, the polynucleotide is on a transmissible genetic element, hence specific embodiments of the invention contemplates a transmissible element comprising a polynucleotide encoding the anti-defense system polypeptide.

As used herein the term “transmissible element” or “transmissible genetic element”, which are interchangeably used, refers to a nucleic acid sequence that allows the transfer of the polynucleotide from one cell to another, e.g. a plasmid.

According to specific embodiments, the construct comprises a recombination element for integrating the polynucleotide into a genome of a phage transfected with the construct.

According to specific embodiments, the nucleic acid sequence heterologous to the polynucleotide encoding the anti-defense system polypeptide is a recombination element.

According to a specific embodiment, the nucleic acid construct comprises a plurality of cloning sites for ligating a nucleic acid sequence of the invention such that it is under transcriptional regulation of the regulatory elements.

Selectable marker genes that ensure maintenance of the construct in the cell can also be included in the construct.

According to specific embodiments, the nucleic acid sequence heterologous to the polynucleotide encoding the anti-defense system polypeptide is a selectable marker.

Preferred selectable markers include those which confer resistance to drugs such as ampicillin, chloramphenicol, erythromycin, kanamycin (neomycin), and tetracycline (Davies et al. (1978) Annu. Rev. Microbiol. 32:469). Selectable markers can also allow a cell to grow on minimal medium, or in the presence of toxic metabolite and can include biosynthetic genes, such as those in the histidine, tryptophan, and leucine biosynthetic pathways.

Where appropriate, the nucleic acid sequences may be optimized for increased expression in the transformed organism. For example, the nucleic acid sequences can be synthesized using preferred codons for improved expression.

The present invention also contemplates uses of the phages disclosed herein. Hence, the phages disclosed herein comprising the polynucleotide encoding an anti-defense system polypeptide can be used for infecting a bacteria and/or treating a bacterial infection.

Thus, according to an aspect of the present invention there is provided a method comprising contacting the bacteria with the genetically modified phage disclosed herein, thereby infecting the bacteria.

According to specific embodiments, the contacting is effected in-vitro or ex-vivo.

According to other specific embodiments, the contacting is effected in-vivo.

According to an additional or an alternative aspect of the present invention there is provided a method of treating a bacterial infection in a subject in need thereof, the method comprising administering to the subject a therapeutically effective amount of a phage comprising a polynucleotide encoding an anti-defense system polypeptide, thereby treating the bacterial infection in the subject.

According to an additional or an alternative aspect of the present invention there is provided a phage comprising a polynucleotide encoding an anti-defense system polypeptide, for use in treating a bacterial infection in the subject in need thereof According to the treatment aspects, the phage may be a native phage comprising a polynucleotide encoding an anti-defense system polypeptide or a genetically modified phage, as further described herein.

As used herein, the term “treating” refers to curing, reversing, attenuating, alleviating, minimizing, suppressing or halting the deleterious effects of a bacterial infection. Those of skill in the art will understand that various methodologies and assays can be used to assess the development of bacterial infection, and similarly, various methodologies and assays may be used to assess the reversal, attenuation, alleviation or suppression of the pathology. Thus, for example, bacterial infection may be assessed by, but not limited to, clinical evaluation, urine dipstick tests, throat culture, sputum tests, histology, indirect non-culture-based tests, including C-reactive protein and procalcitonin tests, serological tests and/or nucleic acid amplification tests.

As used herein, the phrase “subject in need thereof” includes mammals, preferably human beings of any gender and at any age which suffer from bacterial infection.

According to specific embodiments, the bacteria is a Gram-negative bacteria or Negativicutes that stain negative in Gram stain.

Non-limiting examples of Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

According to specific embodiments, the bacteria is gammaproteobacteria (e.g. Escherichia coli, pseudomonas, vibrio and klebsiella) or a Firmicutes (belonging to class Negativicutes that stain negative in Gram stain).

Non-limiting examples of Gram-positive bacteria include, but are not limited to, Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

According to specific embodiments the bacteria is a species selected from the group consisting of Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, Brucella, Campylobacter, Klebsiella, Frankia, Bartonella, Rickettsia, Shewanella, Serratia, Enterobacter, Proteus, Providencia, Brochothrix, and Brevibacterium.

According to specific embodiments, the bacteria is selected from the group consisting of Escherichia coli, Klebsiella pneumoniae, Clostridium difficile, Pseudomonas aeruginosa.

According to specific embodiments, the bacteria expresses the bacterial defense system the anti-defense system polypeptide is directed against e.g. Thoeris, Hachiman, Gabija and/or DSR2.

According to specific embodiments, the method comprises determining expression of the bacterial defense system in the bacteria prior to the contacting or the treating.

Methods of determining expression are well known in the art and include e.g. sequencing, PCR, Western blot etc.

The phage therapy of some embodiments of the invention may be combined with one or more non-phage therapeutic and/or prophylactic agents, useful for the treatment and/or prevention of bacterial infections, as described herein and/or known in the art (e.g. one or more traditional antibiotic agents). Other therapeutic and/or prophylactic agents that may be used in combination with the phage(s) of some embodiments of the invention include, but are not limited to, antibiotic agents, anti-inflammatory agents, antiviral agents, antifungal agents, or local anesthetic agents.

Thus, according to specific embodiments, the methods of the present invention further comprise administering to the subject a therapeutically effective amount of an antibiotic or contacting the bacteria with an antibiotic.

According to specific embodiments, the uses of the present invention further comprise an antibiotic.

Exemplary antibiotics include, but are not limited to aminoglycoside antibiotics, cephalosporins, quinolone antibiotics, macrolide antibiotics, penicillins, sulfonamides, tetracyclines and carbapenems.

Standard or traditional antibiotic agents that can be administered with the phages described herein include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef. ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof.

According to another aspect there is provided an article of manufacture or a kit comprising as active ingredients a phage comprising a polynucleotide encoding an anti-defense system polypeptide; and an antibiotic.

According to specific embodiments, the article of manufacture is identified for treating a bacterial infection.

According to specific embodiments, the phage and the antibiotic are in separate formulations.

Thus, according to specific embodiments, the phage and the antibiotic are packaged in separate containers.

According to yet other specific embodiments the phage and the antibiotic are in a co-formulation.

According to other specific embodiments, the phage therapy is the only active agent administered to the subject, e.g. in the absence of a standard or traditional effective antibiotic agent.

The phages and antibiotic describe herein may be used per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the bacteriophage and/or antibiotic accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient. In one embodiment, the phage may be administered directly into the an infected area or tissue of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping, spray drying, coating or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (phage, antibiotic) effective to prevent, alleviate or ameliorate symptoms of a disorder (i.e. bacterial infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

In some embodiments, the pharmaceutical composition is delivered to a subject in need thereof so as to provide one or more phages in an amount corresponding to a multiplicity of infection (MOI) of about 0.001 to about 10. MOI is determined by assessing the approximate bacterial load, or using an estimate for a given type of infection; and then providing phage in an amount calculated to give the desired MOL.

According to a specific embodiment the composition comprises at least about 10⁶ PFU, 10⁷ PFU, 108 PFU, 10⁹ PFU, or even 10¹⁰ PFU or more of the phage disclosed herein.

In other embodiments, the amount of phage is provided so as to reduce the amount of bacteria by at least 10%, 20%, 30%, 40%, 50%, 60%, 70%, 80%, 90% or even 100%.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that since the phages of embodiments of this invention may enhance the anti-bacterial effect of an antibiotic, doses of the antibiotic may be lower (e.g. 20% lower, 30% lower, 40% lower, 50% lower, 60% lower, 70% lower, 80% lower or even 90% lower) than their gold standard dose or in a sub-efficacious dose when administered as a single agent.

Compositions described herein be comprise a single phage strain or a cocktail of multiple distinct phages wherein as least one of the phages is the phage disclosed herein.

According to specific embodiments, the compositions described herein comprise more than one phage strain. In one embodiment, the composition comprises 2 phage strains, 3 phage strains, 4 phage strains, 5 phage strains or more.

The phage cocktails of some embodiments comprise phages that target a single bacteria species or subspecies.

According to other specific embodiments, the phage cocktail comprises phages that target multiple bacteria species or subspecies, each phage with a distinct host range.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The phages, phage cocktails and articles of manufacture of some embodiments of the invention can be also used in anti-infective compositions for controlling the growth of bacteria on a surface contacted therewith. Thus, the phages of the invention may be incorporated into compositions that are formulated for application to biological surfaces, such as the skin and mucus membranes, as well as for application to non-biological surfaces.

Anti-infective formulations for use on biological surfaces include, but are not limited to, gels, creams, ointments, sprays, and the like. In particular embodiments, the anti-infective formulation is used to sterilize a surgical field, or the hands and/or exposed skin of healthcare workers and/or patients.

Anti-infective formulations for use on non-biological surfaces include sprays, solutions, suspensions, wipes impregnated with a solution or suspension and the like. In particular embodiments, the anti-infective formulation is used on solid surfaces in hospitals, nursing homes, ambulances, etc., including, e.g., appliances, countertops, and medical devices, hospital equipment. In preferred embodiments, the non-biological surface is a surface of a hospital apparatus or piece of hospital equipment. In particularly preferred embodiments, the non-biological surface is a surgical apparatus or piece of surgical equipment.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

TABLE 6
TAD1 IMG homologs

| | DNA | AA |
|---|---|---|
| | sequence | sequence |
| | SEQ ID | SEQ ID |
| Species | NO | NO |
| Clostridioides mangenotii | 185 | 376 |
| Acinetobacter pittii | 170 | 539 |
| Acinetobacter baumannii | 205 | 473 |
| Acinetobacter baumannii | 205 | 473 |
| Clostridium botulinum | 322 | 323 |
| Acinetobacter baumannii | 205 | 473 |
| Acinetobacter baumannii | 94 | 460 |
| Acinetobacter baumannii | 205 | 473 |
| Clostridioides difficile | 281 | 338 |
| Acinetobacter baumannii | 198 | 473 |
| Acinetobacter baumannii | 94 | 460 |
| Acinetobacter baumannii | 168 | 542 |
| Acinetobacter baumannii | 94 | 460 |
| Acinetobacter baumannii | 198 | 473 |
| Acinetobacter calcoaceticus | 170 | 539 |
| Bacillus virus Bcp1 | 271 | 334 |
| Acinetobacter baumannii | 168 | 542 |
| unclassified | 205 | 473 |
| Clostridium botulinum | 76 | 392 |
| Acinetobacter baumannii | 205 | 473 |
| Acinetobacter baumannii | 173 | 540 |
| Clostridium botulinum | 306 | 573 |
| Acinetobacter baumannii | 199 | 473 |
| Bacillus virus Riley | 139 | 552 |
| Tenacibaculum ovolyticum | 46 | 441 |
| Acinetobacter baumannii | 205 | 473 |
| Acinetobacter baumannii | 205 | 473 |
| Clostridium botulinum | 78 | 403 |
| Acinetobacter pittii | 125 | 459 |
| Acinetobacter baumannii | 205 | 473 |
| Acinetobacter baumannii | 200 | 476 |
| Acinetobacter baumannii | 205 | 473 |
| Opitutaceae bacterium TAV5 | 53 | 404 |
| Acinetobacter baumannii | 199 | 473 |
| Clostridium botulinum | 322 | 323 |
| Clostridium botulinum | 322 | 323 |
| Acinetobacter baumannii | 198 | 473 |
| Clostridium botulinum | 305 | 575 |
| Acinetobacter baumannii | 194 | 467 |
| Acinetobacter baumannii | 205 | 473 |
| Clostridium sporogenes | 70 | 389 |
| Acinetobacter baumannii | 198 | 473 |
| Acinetobacter baumannii | 94 | 460 |
| Acinetobacter baumannii | 94 | 460 |
| Acinetobacter pittii | 205 | 473 |
| Acinetobacter pittii | 194 | 467 |
| Acinetobacter baumannii | 94 | 460 |
| Acinetobacter baumannii | 198 | 473 |
| Acinetobacter baumannii | 205 | 473 |
| Acinetobacter baumannii | 198 | 473 |
| Clostridium botulinum | 304 | 574 |
| Acinetobacter baumannii | 94 | 460 |
| Acinetobacter baumannii | 194 | 467 |
| unclassified | 205 | 473 |
| Acinetobacter baumannii | 205 | 473 |
| Acinetobacter baumannii | 199 | 473 |
| Acinetobacter baumannii | 199 | 473 |
| Runella limosa | 209 | 482 |
| Ruminococcus sp. NK3A76 | 161 | 490 |
| Acinetobacter baumannii | 199 | 473 |
| Methylobacterium sp. | 237 | 484 |
| Acinetobacter baumannii | 205 | 473 |
| Agrobacterium sp. SUL3 | 214 | 527 |
| Acinetobacter baumannii | 205 | 473 |
| Acinetobacter baumannii | 205 | 473 |
| Bacillus virus JBP901 | 274 | 336 |
| Acinetobacter baumannii | 205 | 473 |
| Acinetobacter baumannii | 205 | 473 |
| Bacillus virus B4 | 133 | 556 |
| Burkholderia stagnalis | 234 | 530 |
| Acinetobacter baumannii | 197 | 473 |
| Acinetobacter baumannii | 199 | 473 |
| Clostridium botulinum | 85 | 400 |
| Clostridium haemolyticum | 190 | 481 |
| Acinetobacter baumannii | 94 | 460 |
| Acinetobacter baumannii | 198 | 473 |
| Acinetobacter pittii | 170 | 539 |
| Acinetobacter baumannii | 94 | 460 |
| Acinetobacter baumannii | 205 | 473 |
| Acinetobacter pittii | 205 | 473 |
| Bacillus virus Spock | 132 | 555 |
| Acinetobacter pittii | 194 | 467 |
| Acinetobacter baumannii | 205 | 473 |
| Acinetobacter baumannii | 94 | 460 |
| Acinetobacter baumannii | 205 | 473 |
| Acinetobacter baumannii | 205 | 473 |
| Acinetobacter baumannii | 205 | 473 |
| Acinetobacter baumannii | 195 | 468 |
| Acinetobacter baumannii | 205 | 473 |
| Clostridium sp. Marseille-P299 | 162 | 488 |
| Pseudomonas linyingensis | 174 | 543 |
| Acinetobacter baumannii | 205 | 473 |
| Acinetobacter baumannii | 94 | 460 |
| Clostridium botulinum | 85 | 400 |
| Acinetobacter baumannii | 198 | 473 |
| Acinetobacter baumannii | 205 | 473 |
| Acinetobacter baumannii | 94 | 460 |
| Acinetobacter baumannii | 205 | 473 |
| Acinetobacter baumannii | 205 | 473 |
| Acinetobacter baumannii | 199 | 473 |
| Bacillus cereus | 49 | 438 |
| Acinetobacter baumannii | 168 | 542 |
| Clostridioides difficile | 281 | 338 |
| Acinetobacter baumannii | 205 | 473 |
| Acinetobacter baumannii | 198 | 473 |
| Acinetobacter pittii | 170 | 539 |
| Acinetobacter baumannii | 198 | 473 |
| Clostridium roseum | 87 | 380 |
| Acinetobacter pittii | 170 | 539 |
| Acinetobacter pittii | 170 | 539 |
| Fibrobacteria bacterium GUT31 | 249 | 372 |
| Acinetobacter baumannii | 200 | 476 |
| Leptolyngbya sp. PCC 7375 | 189 | 465 |
| Clostridioides difficile | 280 | 339 |
| Acinetobacter baumannii | 199 | 473 |
| Acinetobacter baumannii | 205 | 473 |
| Acinetobacter baumannii | 94 | 460 |
| Clostridium botulinum | 305 | 575 |
| Bacillus weihaiensis | 124 | 463 |
| Clostridium botulinum | 305 | 575 |
| Acinetobacter baumannii | 205 | 473 |
| Acinetobacter pittii | 170 | 539 |
| Acinetobacter baumannii | 205 | 473 |
| Acinetobacter baumannii | 205 | 473 |
| Clostridium botulinum | 305 | 575 |
| Acinetobacter baumannii | 198 | 473 |
| Clostridium botulinum | 79 | 399 |
| Clostridium botulinum | 81 | 396 |
| Acinetobacter baumannii | 168 | 542 |
| Acinetobacter nosocomialis | 194 | 467 |
| Clostridium botulinum | 322 | 323 |
| Acinetobacter baumannii | 198 | 473 |
| Achromobacter sp. 2789STDY5608606 | 130 | 562 |
| Acinetobacter baumannii | 205 | 473 |
| Clostridium botulinum | 322 | 323 |
| Acinetobacter baumannii | 206 | 472 |
| Clostridium botulinum | 306 | 573 |
| Acinetobacter baumannii | 168 | 542 |
| Acinetobacter baumannii | 202 | 475 |
| Acinetobacter baumannii | 205 | 473 |
| Bacillus cereus | 49 | 438 |
| Acinetobacter baumannii | 204 | 470 |
| Acinetobacter baumannii | 205 | 473 |
| Acinetobacter baumannii | 198 | 473 |
| Bacillus virus Shbh1 | 95 | 406 |
| Clostridium botulinum | 306 | 573 |
| Acinetobacter baumannii | 205 | 473 |
| Clostridioides difficile | 281 | 338 |
| Acinetobacter baumannii | 205 | 473 |
| Acinetobacter baumannii | 205 | 473 |
| Acinetobacter baumannii | 205 | 473 |
| Acinetobacter baumannii | 94 | 460 |
| Clostridium botulinum | 322 | 323 |
| Acinetobacter baumannii | 94 | 460 |
| Clostridium botulinum | 81 | 396 |
| Acinetobacter baumannii | 205 | 473 |
| Acinetobacter baumannii | 205 | 473 |
| Clostridium botulinum | 322 | 323 |
| Acinetobacter baumannii | 205 | 473 |
| Acinetobacter baumannii | 94 | 460 |
| Burkholderia stagnalis | 235 | 529 |
| Acinetobacter baumannii | 198 | 473 |
| Clostridium botulinum | 306 | 573 |
| Acinetobacter baumannii | 205 | 473 |
| Acinetobacter baumannii | 205 | 473 |
| Acinetobacter baumannii | 94 | 460 |
| Acinetobacter baumannii | 205 | 473 |
| Acinetobacter baumannii | 205 | 473 |
| Acinetobacter baumannii | 205 | 473 |
| Acinetobacter baumannii | 198 | 473 |
| Acinetobacter baumannii | 94 | 460 |
| Acinetobacter baumannii | 168 | 542 |
| Bacillus virus AvesoBmore | 139 | 552 |
| Leptolyngbya sp. PCC 7375 | 188 | 464 |
| Acinetobacter baumannii | 94 | 460 |
| Acinetobacter baumannii | 205 | 473 |
| Acinetobacter nosocomialis | 205 | 473 |
| Clostridium beijerinckii | 86 | 398 |
| Clostridium botulinum | 322 | 323 |
| Acetobacterium wieringae | 288 | 373 |
| Acinetobacter baumannii | 205 | 473 |
| Clostridium botulinum | 306 | 573 |
| Acinetobacter baumannii | 94 | 460 |
| Acinetobacter baumannii | 205 | 473 |
| Acinetobacter baumannii | 205 | 473 |
| Acinetobacter baumannii | 205 | 473 |
| Acinetobacter baumannii | 205 | 473 |
| Clostridium botulinum | 305 | 575 |
| Acinetobacter baumannii | 205 | 473 |
| Acinetobacter baumannii | 205 | 473 |
| Acinetobacter baumannii | 205 | 473 |
| Acinetobacter baumannii | 199 | 473 |
| Acinetobacter baumannii | 198 | 473 |
| Acinetobacter baumannii | 197 | 473 |
| Acinetobacter pittii | 167 | 542 |
| Clostridium botulinum | 306 | 573 |
| Acinetobacter baumannii | 168 | 542 |
| Bacillus cereus | 49 | 438 |
| Acinetobacter baumannii | 205 | 473 |
| Bacillus virus BCP78 | 115 | 450 |
| Acinetobacter baumannii | 194 | 467 |
| Acinetobacter baumannii | 94 | 460 |
| Clostridium botulinum | 306 | 573 |
| Clostridium botulinum | 306 | 573 |
| Clostridium botulinum | 322 | 323 |
| Acinetobacter sp. NRRL B-65365 | 128 | 559 |
| Clostridium botulinum | 75 | 390 |
| Clostridium botulinum | 306 | 573 |
| Acinetobacter baumannii | 94 | 460 |
| Acinetobacter baumannii | 204 | 470 |
| Acinetobacter baumannii | 94 | 460 |
| Acinetobacter pittii | 171 | 538 |
| Acinetobacter baumannii | 205 | 473 |
| Acinetobacter pittii | 170 | 539 |
| Acinetobacter baumannii | 205 | 473 |
| Gammaproteobacteria bacterium | 213 | 531 |
| RIFCSPHIGHO2_12_FULL_63_22 | | |
| Acinetobacter baumannii | 205 | 473 |
| Clostridium botulinum | 306 | 573 |
| Sinorhizobium meliloti | 215 | 528 |
| Clostridium thermopalmarium | 54 | 430 |
| Acetobacterium dehalogenans | 287 | 374 |
| Acinetobacter baumannii | 205 | 473 |
| Acinetobacter baumannii | 205 | 473 |
| Acinetobacter baumannii | 198 | 473 |
| Clostridium botulinum | 305 | 575 |
| unclassified | 205 | 473 |
| Acinetobacter nosocomialis | 194 | 467 |
| Acinetobacter baumannii | 199 | 473 |
| Acinetobacter baumannii | 199 | 473 |
| Acinetobacter baumannii | 205 | 473 |
| Acinetobacter baumannii | 203 | 473 |
| Acinetobacter pittii | 168 | 542 |
| Clostridium botulinum | 83 | 402 |
| Clostridium botulinum | 305 | 575 |
| Acinetobacter baumannii | 205 | 473 |
| Acinetobacter baumannii | 199 | 473 |
| Clostridium botulinum | 305 | 575 |
| Acinetobacter baumannii | 193 | 478 |
| unclassified | 175 | 534 |
| Acinetobacter baumannii | 199 | 473 |
| Acinetobacter pittii | 310 | 535 |
| Clostridium botulinum | 306 | 573 |
| Acinetobacter baumannii | 205 | 473 |
| Acinetobacter baumannii | 198 | 473 |
| Acinetobacter baumannii | 205 | 473 |
| Acinetobacter baumannii | 205 | 473 |
| Mycobacterium mucogenicum | 314 | 576 |
| Acinetobacter baumannii | 170 | 539 |
| Acinetobacter baumannii | 198 | 473 |
| Acinetobacter baumannii | 205 | 473 |
| Acinetobacter baumannii | 205 | 473 |
| Acinetobacter baumannii | 94 | 460 |
| Acinetobacter baumannii | 205 | 473 |
| Acinetobacter baumannii | 198 | 473 |
| Acinetobacter baumannii | 199 | 473 |
| Clostridium botulinum | 321 | 324 |
| Acinetobacter baumannii | 201 | 471 |
| Clostridium botulinum | 305 | 575 |
| Acinetobacter baumannii | 198 | 473 |
| Acinetobacter baumannii | 205 | 473 |
| Acinetobacter baumannii | 94 | 460 |
| Acinetobacter baumannii | 94 | 460 |
| Acinetobacter baumannii | 205 | 473 |
| Clostridium acetobutylicum | 181 | 533 |
| Acinetobacter baumannii | 205 | 473 |
| Acinetobacter baumannii | 205 | 473 |
| Acinetobacter baumannii | 198 | 473 |
| Acinetobacter baumannii | 205 | 473 |
| Acinetobacter pittii | 169 | 541 |
| Acinetobacter baumannii | 197 | 473 |
| Clostridium botulinum | 322 | 323 |
| Acinetobacter baumannii | 205 | 473 |
| Clostridium botulinum | 306 | 573 |
| Acinetobacter baumannii | 205 | 473 |
| Acinetobacter baumannii | 205 | 473 |
| Acinetobacter baumannii | 205 | 473 |
| Clostridium botulinum | 305 | 575 |
| Acinetobacter baumannii | 198 | 473 |
| Acinetobacter baumannii | 205 | 473 |
| Acinetobacter baumannii | 205 | 473 |
| Clostridium perfringens | 279 | 355 |
| Acinetobacter baumannii | 205 | 473 |
| Acinetobacter baumannii | 205 | 473 |
| Acinetobacter baumannii | 198 | 473 |
| Acinetobacter nosocomialis | 194 | 467 |
| Acinetobacter baumannii | 94 | 460 |
| Clostridium botulinum | 322 | 323 |
| Acinetobacter baumannii | 205 | 473 |
| Clostridium botulinum | 322 | 323 |
| Acinetobacter baumannii | 205 | 473 |
| Clostridium botulinum | 322 | 323 |
| Paenibacillus sp. Mc5Re-14 | 282 | 345 |
| Acinetobacter baumannii | 198 | 473 |
| Acinetobacter sp. BMW17 | 140 | 560 |
| Acinetobacter baumannii | 205 | 473 |
| Acinetobacter baumannii | 200 | 476 |
| Clostridium botulinum | 322 | 323 |
| Acinetobacter baumannii | 199 | 473 |
| Paenibacillus sp. PDC88 | 277 | 329 |
| Acinetobacter baumannii | 205 | 473 |
| Acinetobacter pittii | 310 | 535 |
| Clostridium botulinum | 305 | 575 |
| Acinetobacter baumannii | 205 | 473 |
| Clostridium botulinum | 306 | 573 |
| Acinetobacter baumannii | 207 | 469 |
| Acinetobacter baumannii | 205 | 473 |
| Acinetobacter baumannii | 192 | 479 |
| Acinetobacter baumannii | 200 | 476 |
| Acinetobacter baumannii | 205 | 473 |
| Acinetobacter baumannii | 205 | 473 |
| Acinetobacter baumannii | 199 | 473 |
| Acinetobacter baumannii | 205 | 473 |
| Acinetobacter baumannii | 205 | 473 |
| Acinetobacter baumannii | 205 | 473 |
| Acinetobacter baumannii | 94 | 460 |
| Acinetobacter baumannii | 200 | 476 |
| Clostridium botulinum | 322 | 323 |
| Clostridium beijerinckii | 86 | 398 |
| Acinetobacter baumannii | 94 | 460 |
| Sphingomonas sp. Leaf29 | 217 | 526 |
| Acinetobacter baumannii | 94 | 460 |
| Acinetobacter baumannii | 205 | 473 |
| Clostridium botulinum | 80 | 395 |
| Acinetobacter nosocomialis | 205 | 473 |
| Burkholderia sp. GAS332 | 236 | 525 |
| Acinetobacter baumannii | 168 | 542 |
| Bacillus thuringiensis | 51 | 439 |
| Acinetobacter pittii | 205 | 473 |
| Acinetobacter baumannii | 94 | 460 |
| Bacillus cereus | 50 | 438 |
| Paenibacillus kribbensis | 276 | 346 |
| Sphingomonas sp. Leaf16 | 217 | 526 |
| Acinetobacter baumannii | 94 | 460 |
| Acinetobacter baumannii | 199 | 473 |
| Acinetobacter baumannii | 94 | 460 |
| Acinetobacter baumannii | 205 | 473 |
| Clostridium puniceum | 91 | 422 |
| Clostridium sporogenes | 268 | 358 |
| Acinetobacter baumannii | 94 | 460 |
| Acinetobacter baumannii | 205 | 473 |
| Bacillus phage Phrodo | 136 | 557 |
| Acinetobacter baumannii | 205 | 473 |
| Clostridium botulinum | 305 | 575 |
| Acinetobacter baumannii | 205 | 473 |
| Acinetobacter baumannii | 205 | 473 |
| Acinetobacter baumannii | 199 | 473 |
| Ruminococcus albus | 240 | 351 |
| Acinetobacter baumannii | 199 | 473 |
| Acinetobacter pittii | 172 | 537 |
| Acinetobacter baumannii | 205 | 473 |
| Acinetobacter baumannii | 199 | 473 |
| Acinetobacter baumannii | 94 | 460 |
| Acinetobacter baumannii | 205 | 473 |
| Acinetobacter baumannii | 168 | 542 |
| Acinetobacter baumannii | 168 | 542 |
| Clostridium botulinum | 84 | 401 |
| unclassified | 205 | 473 |
| Acinetobacter pittii | 170 | 539 |
| Acinetobacter sp. ZOR0008 | 205 | 473 |
| Acinetobacter baumannii | 198 | 473 |
| Clostridium botulinum | 305 | 575 |
| Acinetobacter baumannii | 199 | 473 |
| Acinetobacter baumannii | 205 | 473 |
| Acinetobacter nosocomialis | 196 | 477 |
| Clostridium botulinum | 85 | 400 |
| Acinetobacter baumannii | 198 | 473 |
| Acinetobacter baumannii | 198 | 473 |
| Bacillus cereus | 49 | 438 |
| Acinetobacter baumannii | 205 | 473 |
| Acinetobacter baumannii | 205 | 473 |
| Acinetobacter baumannii | 198 | 473 |
| Acinetobacter baumannii | 193 | 478 |
| Acinetobacter baumannii | 205 | 473 |
| Acinetobacter baumannii | 205 | 473 |
| Acinetobacter baumannii | 205 | 473 |
| Acinetobacter baumannii | 205 | 473 |
| Acinetobacter baumannii | 205 | 473 |
| Acinetobacter baumannii | 205 | 473 |
| Acinetobacter pittii | 170 | 539 |
| Achromobacter sp. NFACC18-2 | 131 | 561 |
| Acinetobacter baumannii | 205 | 473 |
| Acinetobacter baumannii | 205 | 473 |
| Acinetobacter baumannii | 205 | 473 |
| Acinetobacter baumannii | 94 | 460 |
| sp. NFACC18-2 | 205 | 473 |
| Acinetobacter pittii | 205 | 473 |
| Acinetobacter oleivorans | 208 | 466 |
| Acinetobacter baumannii | 94 | 460 |
| Acinetobacter baumannii | 94 | 460 |
| Acinetobacter baumannii | 94 | 460 |
| Acinetobacter baumannii | 200 | 476 |
| Acinetobacter baumannii | 94 | 460 |
| Acinetobacter baumannii | 205 | 473 |
| Bacillus phage LH-2015 | 272 | 332 |
| Bacillus cereus | 49 | 438 |
| Clostridium botulinum | 322 | 323 |
| Clostridium botulinum | 74 | 394 |
| Bacillus thuringiensis | 52 | 439 |
| Clostridium botulinum | 322 | 323 |
| Acinetobacter baumannii | 205 | 473 |
| unclassified | 191 | 480 |
| Acinetobacter baumannii | 94 | 460 |
| Acinetobacter baumannii | 94 | 460 |
| Acinetobacter baumannii | 94 | 460 |
| Acinetobacter baumannii | 205 | 473 |
| Acinetobacter baumannii | 205 | 473 |
| Bacillus virus Bigbertha | 138 | 553 |
| Clostridium botulinum | 322 | 323 |
| Acinetobacter baumannii | 206 | 472 |
| Gemmata massiliana | 218 | 485 |
| Clostridium botulinum | 321 | 324 |
| Bacillus virus BCP82 | 270 | 333 |
| Acinetobacter baumannii | 94 | 460 |
| Clostridium botulinum | 305 | 575 |
| Acinetobacter baumannii | 198 | 473 |
| Acinetobacter baumannii | 205 | 473 |
| Candidatus Lokiarchaeota archaeon CR_4 | 308 | 544 |
| Acinetobacter baumannii | 199 | 473 |
| Clostridium roseum | 87 | 380 |
| Clostridium botulinum | 305 | 575 |
| Acinetobacter baumannii | 198 | 473 |
| Clostridium botulinum | 71 | 391 |
| Acinetobacter pittii | 168 | 542 |
| Acinetobacter baumannii | 195 | 468 |
| Acinetobacter baumannii | 199 | 473 |
| Acinetobacter baumannii | 198 | 473 |
| Acinetobacter baumannii | 199 | 473 |
| Acinetobacter baumannii | 205 | 473 |
| Acinetobacter baumannii | 205 | 473 |
| Acinetobacter baumannii | 168 | 542 |
| Acinetobacter baumannii | 205 | 473 |
| Acinetobacter baumannii | 94 | 460 |
| Acinetobacter nosocomialis | 200 | 476 |
| Clostridium botulinum | 305 | 575 |
| Acinetobacter baumannii | 205 | 473 |
| Clostridium botulinum | 306 | 573 |
| Acinetobacter baumannii | 205 | 473 |
| Acinetobacter baumannii | 94 | 460 |
| Acinetobacter baumannii | 94 | 460 |
| Clostridium botulinum | 305 | 575 |
| Acinetobacter baumannii | 205 | 473 |
| Acinetobacter baumannii | 198 | 473 |
| Acinetobacter baumannii | 206 | 472 |
| Acinetobacter baumannii | 205 | 473 |
| Acinetobacter baumannii | 94 | 460 |
| Acinetobacter baumannii | 205 | 473 |
| Acinetobacter baumannii | 205 | 473 |
| Acinetobacter baumannii | 198 | 473 |
| Acinetobacter baumannii | 94 | 460 |
| Clostridium botulinum | 321 | 324 |
| Bacillus virus TsarBomba | 114 | 451 |
| Acinetobacter baumannii | 205 | 473 |
| Acinetobacter baumannii | 205 | 473 |
| Ideonella sakaiensis | 211 | 492 |
| Acinetobacter baumannii | 205 | 473 |
| Acinetobacter baumannii | 200 | 476 |
| Acinetobacter baumannii | 205 | 473 |
| Acinetobacter baumannii | 205 | 473 |
| Acinetobacter baumannii | 205 | 473 |
| Paenibacillus polymyxa | 275 | 347 |
| Acinetobacter baumannii | 94 | 460 |
| Acinetobacter baumannii | 198 | 473 |
| Acinetobacter baumannii | 205 | 473 |
| Acinetobacter baumannii | 198 | 473 |
| Acinetobacter baumannii | 94 | 460 |
| Clostridium botulinum | 306 | 573 |
| Acinetobacter baumannii | 199 | 473 |
| Acinetobacter baumannii | 94 | 460 |
| Clostridium botulinum | 77 | 393 |
| Acinetobacter baumannii | 205 | 473 |
| Acinetobacter pittii | 170 | 539 |
| Noviherbaspirillum sp. Root189 | 216 | 493 |
| Acinetobacter baumannii | 198 | 473 |
| Acinetobacter baumannii | 94 | 460 |
| Acinetobacter baumannii | 205 | 473 |
| Spirochaetes bacterium GWB1_59_5 | 278 | 330 |
| Acinetobacter baumannii | 198 | 473 |
| Clostridium botulinum | 85 | 400 |
| Clostridium botulinum | 306 | 573 |
| Acinetobacter baumannii | 205 | 473 |
| Acinetobacter baumannii | 94 | 460 |
| Clostridium beijerinckii | 86 | 398 |
| Psychrobacter cibarius | 165 | 483 |
| Acinetobacter baumannii | 205 | 473 |
| Acinetobacter baumannii | 205 | 473 |
| Acinetobacter baumannii | 205 | 473 |
| Acinetobacter baumannii | 205 | 473 |
| Clostridium botulinum | 305 | 575 |
| Acinetobacter baumannii | 94 | 460 |
| Acinetobacter sp. | 168 | 542 |
| Acinetobacter baumannii | 94 | 460 |
| Clostridium botulinum | 316 | 577 |
| Bacillus cereus | 49 | 438 |
| Acinetobacter baumannii | 205 | 473 |
| Acinetobacter baumannii | 94 | 460 |
| Bacillus cereus | 49 | 438 |
| Acinetobacter baumannii | 199 | 473 |
| Fibrobacter sp. UWCM | 89 | 379 |
| Achromobacter sp. 2789STDY5608615 | 129 | 563 |
| Clostridioides mangenotii | 269 | 337 |
| Acinetobacter baumannii | 198 | 473 |
| Bacillus virus Troll | 137 | 554 |
| Acinetobacter baumannii | 205 | 473 |
| Acinetobacter baumannii | 179 | 536 |
| Clostridium acetobutylicum | 181 | 533 |
| Acinetobacter baumannii | 205 | 473 |
| Acinetobacter baumannii | 168 | 542 |
| Acinetobacter baumannii | 168 | 542 |
| Acinetobacter baumannii | 94 | 460 |
| Acinetobacter baumannii | 94 | 460 |
| Acinetobacter baumannii | 205 | 473 |
| Acinetobacter baumannii | 198 | 473 |
| Candidatus Amesbacteria bacterium | 312 | 445 |
| GW2011_GWB1_47_19 | | |
| Acinetobacter pittii | 168 | 542 |
| Acinetobacter baumannii | 205 | 473 |
| Clostridium botulinum | 74 | 394 |
| Acinetobacter baumannii | 205 | 473 |
| Acinetobacter baumannii | 94 | 460 |
| Sphingomonas sp. Leaf32 | 217 | 526 |
| Acinetobacter pittii | 168 | 542 |
| Clostridium botulinum | 306 | 573 |
| Acinetobacter baumannii | 205 | 473 |
| Clostridium botulinum | 82 | 397 |
| Acinetobacter baumannii | 198 | 473 |
| Acinetobacter baumannii | 205 | 473 |
| Acinetobacter nosocomialis | 194 | 467 |
| Acinetobacter baumannii | 199 | 473 |
| Clostridium botulinum | 304 | 574 |
| Bacillus krulwichiae | 317 | 325 |
| Acinetobacter pittii | 170 | 539 |
| Acinetobacter baumannii | 198 | 473 |
| Clostridium botulinum | 306 | 573 |
| Acinetobacter baumannii | 94 | 460 |
| Acinetobacter baumannii | 205 | 473 |
| Sporomusa ovata | 126 | 449 |
| Bacillus virus Bc431 | 273 | 335 |
| unclassified | 286 | 326 |

TABLE 7
TAD1 MGV homologs

| | | | | | | | | | | DNA | AA |
|---|---|---|---|---|---|---|---|---|---|---|---|
| | | | | | | | | | | sequence | sequence |
| | | | | | | | | | | SEQ | SEQ |
| ictv_order | ictv_family | ictv_genus | host_domain | host_phylum | host_class | host_order | host_family | host_genus | host_species | ID NO | ID NO |
| Caudovirales | | | | | | | | | | 73 | 382 |
| Caudovirales | | | Bacteria | Firmicutes_A | Clostridia | Oscillospirales | Oscillospiraceae | CAG-110 | GUT_GENOME025308 | 88 | 388 |
| Caudovirales | Myoviridae | | Bacteria | Firmicutes_A | Clostridia | Lachnospirales | Lachnospiraceae | UBA11774 | GUT_GENOME009178 | 187 | 378 |
| Caudovirales | | | | | | | | | | 187 | 378 |
| Caudovirales | | | Bacteria | Firmicutes_A | Clostridia | Oscillospirales | Oscillospiraceae | CAG-110 | GUT_GENOME125250 | 108 | 386 |
| Caudovirales | | | Bacteria | Firmicutes_A | Clostridia | Oscillospirales | Oscillospiraceae | CAG-110 | GUT_GENOME125250 | 108 | 386 |
| Caudovirales | | | Bacteria | Firmicutes_A | Clostridia | Oscillospirales | Oscillospiraceae | CAG-110 | GUT_GENOME125250 | 108 | 386 |
| Caudovirales | | | Bacteria | Firmicutes_A | Clostridia | Oscillospirales | Oscillospiraceae | CAG-110 | GUT_GENOME125250 | 108 | 386 |
| Caudovirales | | | Bacteria | Firmicutes_A | Clostridia | Oscillospirales | Oscillospiraceae | CAG-110 | GUT_GENOME125250 | 110 | 384 |
| Caudovirales | | | Bacteria | Firmicutes_A | Clostridia | Oscillospirales | Oscillospiraceae | CAG-110 | GUT_GENOME125250 | 109 | 387 |
| Caudovirales | | | Bacteria | Firmicutes_A | Clostridia | Oscillospirales | Oscillospiraceae | CAG-110 | GUT_GENOME125250 | 107 | 385 |
| Caudovirales | | | Bacteria | Firmicutes_A | Clostridia | Oscillospirales | Oscillospiraceae | CAG-110 | GUT_GENOME125250 | 113 | 385 |
| Caudovirales | | | Bacteria | Firmicutes_A | Clostridia | Oscillospirales | Oscillospiraceae | CAG-110 | GUT_GENOME125250 | 107 | 385 |
| Caudovirales | | | Bacteria | Firmicutes_A | Clostridia | Oscillospirales | Oscillospiraceae | CAG-110 | GUT_GENOME125250 | 112 | 385 |
| Caudovirales | | | Bacteria | Firmicutes_A | Clostridia | Oscillospirales | Oscillospiraceae | CAG-110 | GUT_GENOME125250 | 111 | 383 |
| Caudovirales | | | Bacteria | Firmicutes_A | Clostridia | Oscillospirales | Oscillospiraceae | CAG-110 | GUT_GENOME125250 | 111 | 383 |
| Caudovirales | | | Bacteria | Firmicutes_A | Clostridia | Oscillospirales | Oscillospiraceae | CAG-110 | GUT_GENOME125250 | 111 | 383 |
| Caudovirales | | | | | | | | | | 72 | 381 |
| Caudovirales | | | Bacteria | Cyanobacteria | Vampirovibrionia | Gastranaerophilales | Gastranaerophilaceae | Zag111 | GUT_GENOME009256 | 145 | 524 |
| Caudovirales | | | | | | | | | | 146 | 523 |
| Caudovirales | | | Bacteria | Cyanobacteria | Vampirovibrionia | Gastranaerophilales | Gastranaerophilaceae | CAG-196 | GUT_GENOME018819 | 180 | 545 |
| Caudovirales | | | Bacteria | Cyanobacteria | Vampirovibrionia | Gastranaerophilales | Gastranaerophilaceae | CAG-196 | GUT_GENOME018819 | 180 | 545 |
| Caudovirales | | | | | | | | | | 311 | 446 |
| Caudovirales | | | | | | | | | | 182 | 375 |
| Caudovirales | | | Bacteria | Firmicutes_A | Clostridia | Oscillospirales | Oscillospiraceae | ER4 | GUT_GENOME258268 | 225 | 510 |
| Caudovirales | | | Bacteria | Firmicutes_A | Clostridia | Oscillospirales | Oscillospiraceae | ER4 | GUT_GENOME258268 | 225 | 510 |
| Caudovirales | Myoviridae | | Bacteria | Firmicutes_A | Clostridia | 4C28d-15 | CAG-727 | | GUT_GENOME166385 | 239 | 350 |
| Caudovirales | | | Bacteria | Firmicutes_A | Clostridia | | | | | 315 | 340 |
| Caudovirales | | | Bacteria | Firmicutes_A | Clostridia | Oscillospirales | | | | 176 | 548 |
| Caudovirales | | | Bacteria | Firmicutes_A | Clostridia | Oscillospirales | | | | 178 | 547 |
| Caudovirales | | | Bacteria | Firmicutes_A | Clostridia | Oscillospirales | | | | 177 | 547 |
| Caudovirales | Siphoviridae | | Bacteria | Firmicutes_A | Clostridia | 4C28d-15 | CAG-727 | QALS01 | GUT_GENOME009249 | 134 | 550 |
| Caudovirales | Siphoviridae | | Bacteria | Firmicutes_A | Clostridia | Oscillospirales | Oscillospiraceae | ER4 | | 232 | 512 |
| Caudovirales | Siphoviridae | | Bacteria | Firmicutes_A | Clostridia | Oscillospirales | Oscillospiraceae | ER4 | | 220 | 499 |
| Caudovirales | Siphoviridae | | Bacteria | Firmicutes_A | Clostridia | Oscillospirales | Oscillospiraceae | ER4 | | 232 | 512 |
| Caudovirales | Siphoviridae | | Bacteria | Firmicutes_A | Clostridia | Oscillospirales | Oscillospiraceae | ER4 | GUT_GENOME258268 | 230 | 512 |
| Caudovirales | Siphoviridae | | Bacteria | Firmicutes_A | Clostridia | Oscillospirales | Oscillospiraceae | ER4 | GUT_GENOME258268 | 230 | 512 |
| Caudovirales | Siphoviridae | | Bacteria | Firmicutes_A | Clostridia | Oscillospirales | Oscillospiraceae | ER4 | | 219 | 504 |
| Caudovirales | Siphoviridae | | | | | | | | | 93 | 453 |
| Caudovirales | | | Bacteria | Firmicutes_A | Clostridia | Oscillospirales | Oscillospiraceae | CAG-170 | GUT_GENOME007990 | 248 | 341 |
| Caudovirales | | | Bacteria | Firmicutes_A | Clostridia | Oscillospirales | Oscillospiraceae | CAG-170 | | 248 | 341 |
| Caudovirales | Siphoviridae | | Bacteria | Firmicutes_A | Clostridia | Oscillospirales | Oscillospiraceae | ER4 | | 231 | 511 |
| Caudovirales | | | Bacteria | Firmicutes_A | Clostridia | Oscillospirales | | | | 247 | 343 |
| Caudovirales | | | Bacteria | Firmicutes_A | Clostridia | Oscillospirales | | | | 247 | 343 |
| Caudovirales | | | Bacteria | Firmicutes_A | Clostridia | Oscillospirales | Oscillospiraceae | CAG-170 | GUT_GENOME007990 | 298 | 571 |
| Caudovirales | | | Bacteria | Firmicutes_A | Clostridia | Oscillospirales | Oscillospiraceae | CAG-170 | GUT_GENOME158868 | 247 | 343 |
| Caudovirales | Siphoviridae | | Bacteria | Firmicutes_A | Clostridia | Oscillospirales | Oscillospiraceae | ER4 | | 221 | 505 |
| Caudovirales | | | Bacteria | Firmicutes_A | Clostridia | Oscillospirales | Oscillospiraceae | ER4 | GUT_GENOME258268 | 223 | 509 |
| Caudovirales | Siphoviridae | | Bacteria | Firmicutes_A | Clostridia | Oscillospirales | Oscillospiraceae | ER4 | | 222 | 505 |
| Caudovirales | Siphoviridae | | Bacteria | Firmicutes_A | Clostridia | Oscillospirales | Oscillospiraceae | ER4 | GUT_GENOME258268 | 223 | 509 |
| Caudovirales | | | Bacteria | Firmicutes_A | Clostridia | Oscillospirales | Oscillospiraceae | ER4 | GUT_GENOME258268 | 221 | 505 |
| Caudovirales | Siphoviridae | | Bacteria | Firmicutes_A | Clostridia | Oscillospirales | Oscillospiraceae | ER4 | | 222 | 505 |
| Caudovirales | | | | | | | | | | 261 | 366 |
| Caudovirales | | | | | | | | | | 265 | 369 |
| Caudovirales | | | Bacteria | Firmicutes_A | Clostridia | Oscillospirales | Oscillospiraceae | | | 301 | 565 |
| Caudovirales | Siphoviridae | | Bacteria | Firmicutes_A | Clostridia | Lachnospirales | Lachnospiraceae | Eisenbergiella | GUT_GENOME239799 | 289 | 567 |
| Caudovirales | Siphoviridae | | Bacteria | Firmicutes_A | Clostridia | Oscillospirales | Ruminococcaceae | Faecalibacterium | | 37 | 420 |
| Caudovirales | | | Bacteria | Firmicutes_A | Clostridia | Oscillospirales | Oscillospiraceae | | | 301 | 565 |
| Caudovirales | Siphoviridae | | Bacteria | Firmicutes_A | Clostridia | Oscillospirales | Oscillospiraceae | CAG-170 | GUT_GENOME007990 | 284 | 328 |
| Caudovirales | | | Bacteria | Firmicutes_A | Clostridia | Oscillospirales | Oscillospiraceae | ER4 | | 227 | 513 |
| Caudovirales | Siphoviridae | | Bacteria | Firmicutes_A | Clostridia | Oscillospirales | Oscillospiraceae | ER4 | | 227 | 513 |
| Caudovirales | | | Bacteria | Firmicutes_A | Clostridia | 4C28d-15 | CAG-727 | UBA10281 | GUT_GENOME269798 | 241 | 349 |
| Caudovirales | | | | | | | | | | 259 | 362 |
| Caudovirales | | | | | | | | | | 266 | 357 |
| Caudovirales | | | | | | | | | | 252 | 360 |
| Caudovirales | Siphoviridae | | | | | | | | | 256 | 361 |
| Caudovirales | | | | | | | | | | 260 | 362 |
| Caudovirales | | | | | | | | | | 262 | 367 |
| Caudovirales | | | | | | | | | | 253 | 360 |
| Caudovirales | | | | | | | | | | 257 | 362 |
| Caudovirales | | | | | | | | | | 262 | 367 |
| Caudovirales | | | | | | | | | | 183 | 447 |
| Caudovirales | | | | | | | | | | 183 | 447 |
| Caudovirales | | | Bacteria | Firmicutes_A | Clostridia | Oscillospirales | Oscillospiraceae | CAG-170 | GUT_GENOME007990 | 290 | 569 |
| Caudovirales | | | Bacteria | Firmicutes_A | Clostridia | Oscillospirales | Oscillospiraceae | CAG-170 | | 299 | 570 |
| Caudovirales | | | Bacteria | Firmicutes_A | Clostridia | Oscillospirales | Oscillospiraceae | CAG-170 | GUT_GENOME007990 | 293 | 569 |
| Caudovirales | | | | | | | | | | 290 | 569 |
| Caudovirales | Siphoviridae | | Bacteria | Firmicutes_A | Clostridia | Oscillospirales | Oscillospiraceae | CAG-170 | GUT_GENOME007990 | 300 | 566 |
| Caudovirales | Siphoviridae | | Bacteria | Firmicutes_A | Clostridia | Oscillospirales | Oscillospiraceae | CAG-170 | | 294 | 569 |
| Caudovirales | Siphoviridae | | Bacteria | Firmicutes_A | Clostridia | Oscillospirales | Oscillospiraceae | | | 290 | 569 |
| Caudovirales | Siphoviridae | | Bacteria | Firmicutes_A | Clostridia | Oscillospirales | Oscillospiraceae | CAG-170 | | 296 | 569 |
| Caudovirales | Siphoviridae | | Bacteria | Firmicutes_A | Clostridia | Oscillospirales | Oscillospiraceae | | | 291 | 569 |
| Caudovirales | | | Bacteria | Firmicutes_A | Clostridia | Oscillospirales | Oscillospiraceae | CAG-170 | | 297 | 569 |
| Caudovirales | Siphoviridae | | Bacteria | Firmicutes_A | Clostridia | Oscillospirales | Oscillospiraceae | | | 290 | 569 |
| Caudovirales | | | Bacteria | Firmicutes_A | Clostridia | Oscillospirales | Oscillospiraceae | | | 292 | 569 |
| Caudovirales | | | Bacteria | Firmicutes_A | Clostridia | Oscillospirales | Oscillospiraceae | CAG-170 | | 292 | 569 |
| Caudovirales | Siphoviridae | | Bacteria | Firmicutes_A | Clostridia | Oscillospirales | Oscillospiraceae | | | 302 | 572 |
| Caudovirales | | | Bacteria | Firmicutes_A | Clostridia | Oscillospirales | Oscillospiraceae | CAG-170 | GUT_GENOME007990 | 300 | 566 |
| Caudovirales | Siphoviridae | | Bacteria | Firmicutes_A | Clostridia | Oscillospirales | Oscillospiraceae | | | 290 | 569 |
| Caudovirales | Siphoviridae | | Bacteria | Firmicutes_A | Clostridia | Oscillospirales | Oscillospiraceae | | | 291 | 569 |
| Caudovirales | Siphoviridae | | Bacteria | Firmicutes_A | Clostridia | Oscillospirales | Oscillospiraceae | | | 290 | 569 |
| Caudovirales | Siphoviridae | | Bacteria | Firmicutes_A | Clostridia | Oscillospirales | Oscillospiraceae | CAG-170 | | 299 | 570 |
| Caudovirales | | | Bacteria | Firmicutes_A | Clostridia | Oscillospirales | Oscillospiraceae | CAG-170 | | 296 | 569 |
| Caudovirales | | | Bacteria | Firmicutes_A | Clostridia | Oscillospirales | Oscillospiraceae | CAG-170 | | 294 | 569 |
| Caudovirales | Siphoviridae | | Bacteria | Firmicutes_A | Clostridia | Oscillospirales | Oscillospiraceae | | | 302 | 572 |
| Caudovirales | | | Bacteria | Firmicutes_A | Clostridia | Oscillospirales | Oscillospiraceae | CAG-170 | | 299 | 570 |
| Caudovirales | Siphoviridae | | Bacteria | Firmicutes_A | Clostridia | Oscillospirales | Oscillospiraceae | ER4 | GUT_GENOME258268 | 229 | 508 |
| Caudovirales | Siphoviridae | | Bacteria | Firmicutes_A | Clostridia | Oscillospirales | Oscillospiraceae | ER4 | GUT_GENOME258268 | 229 | 508 |
| Caudovirales | | | Bacteria | Firmicutes_A | Clostridia | 4C28d-15 | | | | 242 | 348 |
| Caudovirales | | | | | | | | | | 242 | 348 |
| Caudovirales | | | Bacteria | Firmicutes_A | Clostridia | Oscillospirales | Oscillospiraceae | CAG-170 | GUT_GENOME007990 | 245 | 342 |
| Caudovirales | | | Bacteria | Firmicutes_A | Clostridia | Oscillospirales | Oscillospiraceae | CAG-170 | | 243 | 342 |
| Caudovirales | | | Bacteria | Firmicutes_A | Clostridia | Oscillospirales | Oscillospiraceae | CAG-170 | | 246 | 342 |
| Caudovirales | | | Bacteria | Firmicutes_A | Clostridia | Oscillospirales | Oscillospiraceae | CAG-170 | | 245 | 342 |
| Caudovirales | Siphoviridae | | Bacteria | Firmicutes_A | Clostridia | Oscillospirales | Oscillospiraceae | CAG-170 | GUT_GENOME007990 | 246 | 342 |
| Caudovirales | | | Bacteria | Firmicutes_A | Clostridia | | | | | 245 | 342 |
| Caudovirales | | | Bacteria | Firmicutes_A | Clostridia | Oscillospirales | Oscillospiraceae | CAG-170 | GUT_GENOME007990 | 285 | 327 |
| Caudovirales | | | Bacteria | Firmicutes_A | Clostridia | Oscillospirales | Oscillospiraceae | CAG-170 | | 244 | 342 |
| Caudovirales | | | Bacteria | Firmicutes_A | Clostridia | Oscillospirales | Oscillospiraceae | ER4 | | 224 | 507 |
| Caudovirales | Siphoviridae | | Bacteria | Firmicutes_A | Clostridia | Oscillospirales | Oscillospiraceae | ER4 | GUT_GENOME258268 | 233 | 500 |
| Caudovirales | | | Bacteria | Firmicutes_A | Clostridia | Oscillospirales | Oscillospiraceae | ER4 | | 224 | 507 |
| Caudovirales | | | | | | | | | | 36 | 421 |
| Caudovirales | | | Bacteria | Firmicutes_A | Clostridia | Oscillospirales | Oscillospiraceae | ER4 | | 228 | 506 |
| Caudovirales | Siphoviridae | | Bacteria | Firmicutes_A | Clostridia | Oscillospirales | Oscillospiraceae | ER4 | | 226 | 503 |
| Caudovirales | | | | | | | | | | 258 | 364 |
| Caudovirales | | | | | | | | | | 267 | 356 |
| Caudovirales | | | | | | | | | | 255 | 371 |
| Caudovirales | | | | | | | | | | 92 | 454 |
| Caudovirales | | | | | | | | | | 263 | 365 |
| Caudovirales | Myoviridae | | | | | | | | | 135 | 551 |
| Caudovirales | | | Bacteria | Firmicutes_A | Clostridia | Oscillospirales | Oscillospiraceae | CAG-170 | GUT_GENOME007990 | 295 | 568 |
| Caudovirales | | | Bacteria | Firmicutes_A | Clostridia | Oscillospirales | Oscillospiraceae | CAG-170 | GUT_GENOME007990 | 295 | 568 |
| Caudovirales | | | Bacteria | Firmicutes_A | Clostridia | Oscillospirales | Oscillospiraceae | Oscillibacter | GUT_GENOME124777 | 144 | 496 |
| Caudovirales | Siphoviridae | | | | | | | | | 48 | 443 |
| Caudovirales | Siphoviridae | | | | | | | | | 264 | 359 |
| Caudovirales | | | | | | | | | | 250 | 363 |
| Caudovirales | | | | | | | | | | 251 | 368 |
| Caudovirales | | | | | | | | | | 47 | 442 |
| Caudovirales | | | | | | | | | | 47 | 442 |
| Caudovirales | Siphoviridae | | | | | | | | | 264 | 359 |
| Caudovirales | Siphoviridae | | Bacteria | Patescibacteria | Saccharimonadia | Saccharimonadales | Saccharimonadaceae | TM7x | GUT_GENOME063140 | 47 | 442 |
| Caudovirales | | | | | | | | | | 307 | 546 |
| Caudovirales | | | | | | | | | | 45 | 440 |
| Caudovirales | | Triavirus | Bacteria | Firmicutes_A | Clostridia | Lachnospirales | Lachnospiraceae | | | 42 | 409 |
| Caudovirales | | | Bacteria | Firmicutes_A | Clostridia | Lachnospirales | Lachnospiraceae | | | 42 | 409 |
| Caudovirales | | | Bacteria | Firmicutes_A | Clostridia | Lachnospirales | Lachnospiraceae | | | 65 | 435 |
| Caudovirales | Siphoviridae | | Bacteria | Firmicutes_A | Clostridia | Lachnospirales | Lachnospiraceae | Dorea | GUT_GENOME000149 | 41 | 414 |
| Caudovirales | | | Bacteria | Firmicutes_A | Clostridia | Lachnospirales | Lachnospiraceae | | | 39 | 411 |
| Caudovirales | | | Bacteria | Firmicutes_A | Clostridia | Lachnospirales | Lachnospiraceae | | | 65 | 435 |
| Caudovirales | | | Bacteria | Firmicutes_A | Clostridia | Lachnospirales | Lachnospiraceae | Dorea | GUT_GENOME000149 | 40 | 417 |
| Caudovirales | | | Bacteria | Firmicutes_A | Clostridia | Lachnospirales | Lachnospiraceae | Dorea | GUT_GENOME000149 | 39 | 411 |
| Caudovirales | | | Bacteria | Firmicutes_A | Clostridia | Lachnospirales | Lachnospiraceae | Dorea | GUT_GENOME000149 | 39 | 411 |
| Caudovirales | | | Bacteria | Firmicutes_A | Clostridia | Lachnospirales | Lachnospiraceae | Dorea | GUT_GENOME000149 | 40 | 417 |
| Caudovirales | | | Bacteria | Firmicutes_A | Clostridia | Lachnospirales | Lachnospiraceae | Dorea | GUT_GENOME000149 | 40 | 417 |
| Caudovirales | | | Bacteria | Firmicutes_A | Clostridia | Lachnospirales | Lachnospiraceae | | | 43 | 408 |
| Caudovirales | Siphoviridae | | Bacteria | Firmicutes_A | Clostridia | Lachnospirales | Lachnospiraceae | Clostridium_M | | 164 | 489 |
| Caudovirales | Siphoviridae | | Bacteria | Firmicutes_A | Clostridia | Lachnospirales | Lachnospiraceae | Clostridium_M | | 166 | 491 |
| Caudovirales | | | Bacteria | Firmicutes_A | Clostridia | Lachnospirales | Lachnospiraceae | Dorea | GUT_GENOME000149 | 64 | 434 |
| Caudovirales | | | Bacteria | Firmicutes_A | Clostridia | Lachnospirales | Lachnospiraceae | | | 97 | 415 |
| Caudovirales | | | | | | | | | | 35 | 405 |
| Caudovirales | | | | | | | | | | 238 | 344 |
| Caudovirales | Siphoviridae | | Bacteria | Firmicutes_A | Clostridia | Lachnospirales | Lachnospiraceae | Faecalicatena | | 68 | 433 |
| Caudovirales | | | Bacteria | Firmicutes_A | Clostridia | Lachnospirales | Lachnospiraceae | | | 66 | 436 |
| Caudovirales | Siphoviridae | | Bacteria | Firmicutes_A | Clostridia | Lachnospirales | Lachnospiraceae | | | 69 | 433 |
| Caudovirales | Siphoviridae | | Bacteria | Firmicutes_A | Clostridia | Lachnospirales | Lachnospiraceae | | | 67 | 437 |
| Caudovirales | | | Bacteria | Firmicutes_A | Clostridia | Lachnospirales | Lachnospiraceae | | | 99 | 412 |
| Caudovirales | | | Bacteria | Firmicutes_A | Clostridia | Lachnospirales | Lachnospiraceae | | | 101 | 412 |
| Caudovirales | | | Bacteria | Firmicutes_A | Clostridia | Lachnospirales | Lachnospiraceae | | | 101 | 412 |
| Caudovirales | Siphoviridae | | Bacteria | Firmicutes_A | Clostridia | Lachnospirales | Lachnospiraceae | | | 63 | 431 |
| Caudovirales | Siphoviridae | | Bacteria | Firmicutes_A | Clostridia | Lachnospirales | Lachnospiraceae | | | 67 | 437 |
| Caudovirales | | | Bacteria | Firmicutes_A | Clostridia | Lachnospirales | Lachnospiraceae | Faecalicatena | | 68 | 433 |
| Caudovirales | | | Bacteria | Firmicutes_A | Clostridia | Lachnospirales | Lachnospiraceae | | | 100 | 412 |
| Caudovirales | | | Bacteria | Firmicutes_A | Clostridia | Lachnospirales | Lachnospiraceae | | | 100 | 412 |
| Caudovirales | | | Bacteria | Firmicutes_A | Clostridia | Lachnospirales | Lachnospiraceae | | | 66 | 436 |
| Caudovirales | Siphoviridae | | Bacteria | Firmicutes_A | Clostridia | Lachnospirales | Lachnospiraceae | | | 69 | 433 |
| Caudovirales | | | Bacteria | Firmicutes_A | Clostridia | Lachnospirales | Lachnospiraceae | | | 63 | 431 |
| Caudovirales | Siphoviridae | | Bacteria | Firmicutes_A | Clostridia | Lachnospirales | Lachnospiraceae | | | 67 | 437 |
| Caudovirales | | | Bacteria | Firmicutes_A | Clostridia | Lachnospirales | Lachnospiraceae | | | 102 | 410 |
| Caudovirales | Siphoviridae | | Bacteria | Firmicutes_A | Clostridia | Lachnospirales | Lachnospiraceae | | | 63 | 431 |
| Caudovirales | | | Bacteria | Firmicutes_A | Clostridia | Lachnospirales | Lachnospiraceae | | | 96 | 416 |
| Caudovirales | Siphoviridae | | Bacteria | Firmicutes_A | Clostridia | Clostridiales | Clostridiaceae | Clostridium | GUT_GENOME096036 | 303 | 564 |
| Caudovirales | Podoviridae | | Bacteria | Cyanobacteria | Vampirovibrionia | Gastranaerophilales | Gastranaerophilaceae | Zag111 | GUT_GENOME009256 | 127 | 549 |
| Caudovirales | | | | | | | | | | 254 | 370 |
| Caudovirales | Myoviridae | | Bacteria | | | | | | | 158 | 498 |
| Caudovirales | | | | | | | | | | 313 | 419 |
| Caudovirales | Siphoviridae | | Bacteria | Firmicutes_A | Clostridia | Oscillospirales | Ruminococcaceae | Ruminococcus_C | | 320 | 353 |
| Caudovirales | | | Bacteria | Firmicutes_A | Clostridia | Lachnospirales | Lachnospiraceae | | | 106 | 432 |
| Caudovirales | Podoviridae | | | | | | | | | 309 | 532 |
| Caudovirales | | | | | | | | | | 120 | 455 |
| Caudovirales | Siphoviridae | | Bacteria | Firmicutes_A | Clostridia | Oscillospirales | Ruminococcaceae | Ruminococcus_C | | 318 | 354 |
| Caudovirales | Siphoviridae | | Bacteria | Firmicutes_A | Clostridia | Oscillospirales | Ruminococcaceae | Ruminococcus_C | | 318 | 354 |
| Caudovirales | Siphoviridae | | Bacteria | Firmicutes_A | Clostridia | Oscillospirales | Ruminococcaceae | Ruminococcus_C | GUT_GENOME066545 | 318 | 354 |
| Caudovirales | Siphoviridae | | Bacteria | Firmicutes_A | Clostridia | Oscillospirales | Ruminococcaceae | Ruminococcus_C | | 318 | 354 |
| Caudovirales | Siphoviridae | | Bacteria | Firmicutes_A | Clostridia | Lachnospirales | Lachnospiraceae | Blautia | | 159 | 515 |
| Caudovirales | Siphoviridae | | Bacteria | Firmicutes_A | Clostridia | Lachnospirales | Lachnospiraceae | Blautia | | 159 | 515 |
| Caudovirales | | | Bacteria | Proteobacteria | Gammaproteobacteria | Pseudomonadales | Moraxellaceae | Acinetobacter | GUT_GENOME141315 | 205 | 474 |
| Caudovirales | | | | | | | | | | 121 | 457 |
| Caudovirales | | | | | | | | | | 117 | 456 |
| Caudovirales | | | | | | | | | | 117 | 456 |
| Caudovirales | | | | | | | | | | 117 | 456 |
| Caudovirales | | | | | | | | | | 119 | 452 |
| Caudovirales | | | | | | | | | | 118 | 458 |
| Caudovirales | | | | | | | | | | 116 | 458 |
| Caudovirales | Siphoviridae | | | | | | | | | 90 | 444 |
| Caudovirales | Myoviridae | | Bacteria | Firmicutes_A | Clostridia | | | | | 142 | 494 |
| Caudovirales | Myoviridae | | Bacteria | | | | | | | 143 | 495 |
| Caudovirales | Siphoviridae | | Bacteria | Firmicutes_A | Clostridia | Lachnospirales | Lachnospiraceae | | | 98 | 418 |
| Caudovirales | Siphoviridae | | Bacteria | Firmicutes_A | Clostridia | Lachnospirales | Lachnospiraceae | | | 98 | 418 |
| Caudovirales | Siphoviridae | | Bacteria | Firmicutes_A | Clostridia | Oscillospirales | Ruminococcaceae | Ruminococcus_C | | 319 | 352 |
| Caudovirales | | | Bacteria | Firmicutes_A | Clostridia | Lachnospirales | Lachnospiraceae | Dorea | GUT_GENOME000149 | 44 | 413 |
| Caudovirales | | | Bacteria | Firmicutes_A | Clostridia | Oscillospirales | Oscillospiraceae | ER4 | | 186 | 377 |
| Caudovirales | | | Bacteria | Firmicutes_A | Clostridia | Lachnospirales | Lachnospiraceae | Agathobacter | GUT_GENOME143712 | 104 | 425 |
| Caudovirales | | | Bacteria | Firmicutes_A | Clostridia | Lachnospirales | Lachnospiraceae | Agathobacter | GUT_GENOME143712 | 62 | 425 |
| Caudovirales | | | Bacteria | Firmicutes_A | Clostridia | Lachnospirales | Lachnospiraceae | Agathobacter | GUT_GENOME143712 | 62 | 425 |
| Caudovirales | | | Bacteria | Firmicutes_A | Clostridia | Lachnospirales | Lachnospiraceae | | | 105 | 425 |
| Caudovirales | | | Bacteria | Firmicutes_A | Clostridia | Lachnospirales | Lachnospiraceae | Agathobacter | GUT_GENOME143712 | 104 | 425 |
| Caudovirales | | | Bacteria | Firmicutes_A | Clostridia | Lachnospirales | Lachnospiraceae | | | 105 | 425 |
| Caudovirales | | | Bacteria | Firmicutes_A | Clostridia | Lachnospirales | Lachnospiraceae | Agathobacter | GUT_GENOME143712 | 62 | 425 |
| Caudovirales | | | Bacteria | Firmicutes_A | Clostridia | Lachnospirales | Lachnospiraceae | Agathobacter | GUT_GENOME143712 | 61 | 424 |
| Caudovirales | | | Bacteria | Firmicutes_A | Clostridia | Lachnospirales | Lachnospiraceae | Agathobacter | GUT_GENOME143712 | 103 | 425 |
| Caudovirales | | | Bacteria | Firmicutes_A | Clostridia | Lachnospirales | Lachnospiraceae | Agathobacter | GUT_GENOME143712 | 62 | 425 |
| Caudovirales | | | Bacteria | Firmicutes_A | Clostridia | Lachnospirales | Lachnospiraceae | Agathobacter | GUT_GENOME143712 | 61 | 424 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | Bacteria | Firmicutes_A | Clostridia | Peptostreptococcales | Peptostreptococcaceae | Intestinibacter | | 163 | 487 |
| Caudovirales | | | | | | | | | | 55 | 423 |
| Caudovirales | | | Bacteria | Firmicutes_A | Clostridia | Clostridiales | Clostridiaceae | Clostridium | GUT_GENOME058212 | 157 | 497 |
| Caudovirales | | | Bacteria | Firmicutes_A | Clostridia | Lachnospirales | Lachnospiraceae | Agathobacter | GUT_GENOME143712 | 184 | 448 |
| Caudovirales | | | Bacteria | Firmicutes_A | Clostridia | Lachnospirales | Lachnospiraceae | Agathobacter | GUT_GENOME143712 | 58 | 426 |
| Caudovirales | | | Bacteria | Firmicutes_A | Clostridia | Lachnospirales | Lachnospiraceae | Agathobacter | GUT_GENOME143712 | 56 | 427 |
| Caudovirales | | | Bacteria | Firmicutes_A | Clostridia | Lachnospirales | Lachnospiraceae | Agathobacter | GUT_GENOME143712 | 58 | 426 |
| Caudovirales | | | Bacteria | Firmicutes_A | Clostridia | Lachnospirales | Lachnospiraceae | Agathobacter | GUT_GENOME143712 | 56 | 427 |
| Caudovirales | | | Bacteria | Firmicutes_A | Clostridia | Lachnospirales | Lachnospiraceae | Agathobacter | GUT_GENOME143712 | 60 | 428 |
| Caudovirales | | | Bacteria | Firmicutes_A | Clostridia | Lachnospirales | Lachnospiraceae | Agathobacter | GUT_GENOME143712 | 59 | 429 |
| Caudovirales | | | Bacteria | Firmicutes_A | Clostridia | Lachnospirales | Lachnospiraceae | Agathobacter | GUT_GENOME143712 | 56 | 427 |
| Caudovirales | | | Bacteria | Firmicutes_A | Clostridia | Lachnospirales | Lachnospiraceae | Agathobacter | GUT_GENOME143712 | 59 | 429 |
| Caudovirales | | | Bacteria | Firmicutes_A | Clostridia | Lachnospirales | Lachnospiraceae | Agathobacter | GUT_GENOME143712 | 58 | 426 |
| Caudovirales | | | Bacteria | Firmicutes_A | Clostridia | Lachnospirales | Lachnospiraceae | Agathobacter | GUT_GENOME143712 | 57 | 426 |
| Caudovirales | | | Bacteria | Firmicutes_A | Clostridia | Lachnospirales | Lachnospiraceae | Agathobacter | GUT_GENOME143712 | 58 | 426 |
| Caudovirales | Siphoviridae | | Bacteria | Firmicutes_A | Clostridia | Peptostreptococcales | Peptostreptococcaceae | Intestinibacter | GUT_GENOME000245 | 122 | 461 |
| Caudovirales | Siphoviridae | | Bacteria | | | | | | | 122 | 461 |
| Caudovirales | | | | | | | | | | 141 | 558 |
| Caudovirales | Siphoviridae | | Bacteria | | | | | | | 123 | 462 |
| Caudovirales | Siphoviridae | | Bacteria | Firmicutes_A | Clostridia | Clostridiales | Clostridiaceae | Clostridium_P | GUT_GENOME142391 | 160 | 514 |
| Caudovirales | Siphoviridae | | Bacteria | Firmicutes_A | Clostridia | | | | | 148 | 516 |
| Caudovirales | Siphoviridae | | Bacteria | Firmicutes_A | Clostridia | Peptostreptococcales | Peptostreptococcaceae | Intestinibacter | GUT_GENOME000245 | 283 | 331 |
| Caudovirales | Siphoviridae | | Bacteria | Firmicutes_A | Clostridia | Peptostreptococcales | Peptostreptococcaceae | | | 212 | 486 |
| Caudovirales | | | Bacteria | Firmicutes_A | Clostridia | Peptostreptococcales | Peptostreptococcaceae | Intestinibacter | | 210 | 502 |
| Caudovirales | Myoviridae | | Bacteria | Firmicutes_A | Clostridia | Peptostreptococcales | Peptostreptococcaceae | Intestinibacter | GUT_GENOME000245 | 156 | 522 |
| Caudovirales | | | Bacteria | Firmicutes_A | Clostridia | Peptostreptococcales | Peptostreptococcaceae | Intestinibacter | GUT_GENOME000245 | 149 | 517 |
| Caudovirales | Siphoviridae | | Bacteria | Firmicutes_A | Clostridia | Peptostreptococcales | Peptostreptococcaceae | Intestinibacter | GUT_GENOME000245 | 147 | 501 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | | | Bacteria | Firmicutes_A | Clostridia | Peptostreptococcales | Peptostreptococcaceae | Intestinibacter | GUT_GENOME000245 | 150 | 518 |
| Caudovirales | Siphoviridae | | Bacteria | Firmicutes_A | Clostridia | Peptostreptococcales | Peptostreptococcaceae | Terrisporobacter | GUT_GENOME000534 | 38 | 407 |
| Caudovirales | | | Bacteria | Firmicutes_A | Clostridia | Peptostreptococcales | Peptostreptococcaceae | Intestinibacter | GUT_GENOME000245 | 151 | 521 |
| Caudovirales | Myoviridae | | Bacteria | Firmicutes_A | Clostridia | Peptostreptococcales | Peptostreptococcaceae | Intestinibacter | GUT_GENOME000245 | 153 | 519 |
| Caudovirales | | | Bacteria | Firmicutes_A | Clostridia | Peptostreptococcales | Peptostreptococcaceae | Intestinibacter | GUT_GENOME000245 | 151 | 521 |
| Caudovirales | Myoviridae | | Bacteria | Firmicutes | Bacilli | RF39 | CAG-1000 | CAG-1000 | GUT_GENOME273237 | 152 | 520 |
| Caudovirales | Myoviridae | | | | | | | | | 153 | 519 |
| Caudovirales | Myoviridae | | Bacteria | Firmicutes | Bacilli | RF39 | CAG-1000 | CAG-1000 | GUT_GENOME273237 | 154 | 519 |
| Caudovirales | Myoviridae | | | | | | | | | 153 | 519 |

TABLE 8
HAD1 IMG homologs

| | DNA sequence | AA sequence |
|---|---|---|
| Species | SEQ ID NO | SEA ID NO |
| Bacillus virus HoodyT | 592 | 605 |
| Paenibacillus pinihumi | 594 | 595 |
| Bacillus toyonensis | 593 | 609 |
| Paenibacillus pinihumi | 594 | 595 |
| Bacillus virus Evoli | 588 | 607 |
| Paenibacillus odorifer | 580 | 603 |
| Bacillus virus Bastille | 587 | 608 |
| Bacillus virus CAM003 | 586 | 606 |
| Bacillus virus Troll | 584 | 600 |
| Fontibacillus phaseoli | 578 | 604 |
| Bacillus phage Phrodo | 582 | 599 |
| Bacillus toyonensis | 593 | 609 |
| Bacillus virus Riley | 583 | 600 |
| Paenibacillus sp. FSL H8-237 | 580 | 603 |
| Paenibacillus sp. FSL R7-269 | 579 | 602 |
| Bacillus virus AvesoBmore | 583 | 600 |
| Bacillus virus BCP78 | 590 | 596 |
| Paenibacillus odorifer | 580 | 603 |
| Bacillus virus Spock | 585 | 601 |
| Bacillus virus B4 | 591 | 610 |
| Bacillus virus Bigbertha | 581 | 598 |
| Bacillus toyonensis | 589 | 597 |

TABLE 9
GAD1 IMG homologs

| | DNA | AA |
|---|---|---|
| | sequence | sequence |
| | SEQ ID | SEQ ID |
| Species | NO | NO |
| Clostridioides difficile | 643 | 719 |
| Bacillus velezensis | 624 | 701 |
| Paenibacillus sp. HJL G12 | 669 | 677 |
| Xenorhabdus griffiniae | 649 | 684 |
| Bacillus subtilis | 622 | 709 |
| Bacillus subtilis | 631 | 703 |
| Bacillus vallismortis | 616 | 694 |
| Bacillus subtilis | 623 | 710 |
| Bacillus amyloliquefaciens | 625 | 700 |
| Proteus sp. G2664 | 651 | 686 |
| Bacillus subtilis | 626 | 698 |
| Cedecea lapagei | 655 | 680 |
| Bacillus subtilis | 621 | 705 |
| Proteus sp. G2664 | 651 | 686 |
| Xenorhabdus sp. KK7.4 | 653 | 682 |
| Bacillus subtilis | 629 | 708 |
| Bacillus velezensis | 615 | 696 |
| Bacillus subtilis | 620 | 706 |
| Bacillus subtilis | 671 | 679 |
| Bacillus subtilis | 628 | 707 |
| unclassified | 620 | 706 |
| Photorhabdus khanii | 652 | 683 |
| Shewanella sp. phage 1/4 | 644 | 726 |
| Bacillus vallismortis | 616 | 694 |
| Bacillus nakamurai | 612 | 716 |
| Bacillus subtilis | 668 | 676 |
| Nitrobacter hamburgensis | 634 | 711 |
| Bacillus amyloliquefaciens | 629 | 708 |
| Proteus sp. G2664 | 651 | 686 |
| Proteus sp. G2670 | 651 | 686 |
| Blautia wexlerae | 663 | 733 |
| Clostridioides difficile | 642 | 720 |
| Bacillus amyloliquefaciens | 629 | 708 |
| Bacillus subtilis | 668 | 676 |
| Lachnospiraceae bacterium Marseille-P3773 | 674 | 675 |
| Enterobacter roggenkampii | 645 | 723 |
| Paenibacillus sp. EKM202P | 672 | 678 |
| Cedecea lapagei | 654 | 681 |
| Xenorhabdus sp. GDc328 | 649 | 684 |
| Pectinatus haikarae | 611 | 715 |
| Bacillus subtilis | 632 | 704 |
| Bacillus amyloliquefaciens | 633 | 693 |
| Proteus sp. G2670 | 651 | 686 |
| Bacillus amyloliquefaciens | 633 | 693 |
| Photorhabdus temperata | 652 | 683 |
| Escherichia coli | 647 | 725 |
| [Clostridium] methoxybenzovorans | 670 | 718 |
| Proteus sp. G2661 | 650 | 685 |
| Proteus sp. G2664 | 651 | 686 |
| Bacillus amyloliquefaciens | 633 | 693 |
| Bacillus subtilis | 630 | 702 |
| Bacillus xiamenensis | 635 | 695 |
| Bacillus amyloliquefaciens | 629 | 708 |
| Bacillus subtilis | 622 | 709 |
| Bacillus camelliae | 659 | 730 |
| Bacillus amyloliquefaciens | 629 | 708 |
| Bacillus subtilis | 648 | 717 |
| Bacillus amyloliquefaciens | 627 | 699 |
| Bacillus amyloliquefaciens | 633 | 693 |
| Bacillus subtilis | 623 | 710 |
| Proteus sp. G2670 | 651 | 686 |
| Proteus sp. G2670 | 651 | 686 |
| Bacillus atrophaeus | 636 | 697 |
| Brevibacillus gelatini | 617 | 689 |
| Clostridioides difficile | 642 | 720 |
| Bacillus inaquosorum | 620 | 706 |

TABLE 10
GAD1 MGV homologs

| | | | | | | | | | | DNA | AA |
|---|---|---|---|---|---|---|---|---|---|---|---|
| | | | | | | | | | | sequence | sequence |
| | | | | | | | | | | SEQ | SEQ |
| ictv_order | ictv_family | ictv_genus | host_domain | host_phylum | host_class | host_order | host_family | host_genus | host_species | ID NO | ID NO |
| Caudovirales | | Light- | | | | | | | | 656 | 727 |
| | | bulb | | | | | | | | | |
| | | virus | | | | | | | | | |
| Caudovirales | Siphoviridae | | Bacteria | Firmicutes_A | Clostridia | Lachnospirales | Lachnospiraceae | | | 662 | 736 |
| Caudovirales | Siphoviridae | | Bacteria | Firmicutes_A | Clostridia | Lachnospirales | Lachnospiraceae | | | 666 | 732 |
| Caudovirales | Siphoviridae | | Bacteria | Firmicutes_A | Clostridia | Lachnospirales | Lachnospiraceae | | | 660 | 737 |
| Caudovirales | | | Bacteria | Proteobacteria | Gammaproteo- | Enterobacterales | Enterobacteriaceae | Escherichia | GUT_GENOME144544 | 646 | 724 |
| | | | | | bacteria | | | | | | |
| Caudovirales | | | Bacteria | Firmicutes_A | Clostridia | Lachnospirales | Lachnospiraceae | | | 673 | 687 |
| Caudovirales | | | Bacteria | Firmicutes_A | Clostridia | Lachnospirales | Lachnospiraceae | | | 673 | 687 |
| Caudovirales | | | Bacteria | Firmicutes_A | Clostridia | Lachnospirales | Lachnospiraceae | | | 664 | 734 |
| Caudovirales | | | Bacteria | Firmicutes_A | Clostridia | Lachnospirales | Lachnospiraceae | | | 664 | 734 |
| Caudovirales | | | Bacteria | Firmicutes_A | Clostridia | Lachnospirales | Lachnospiraceae | | | 664 | 734 |
| Caudovirales | Siphoviridae | | Bacteria | Firmicutes_A | Clostridia | Lachnospirales | Lachnospiraceae | Blautia_A | | 657 | 729 |
| Caudovirales | Siphoviridae | | Bacteria | Firmicutes_A | Clostridia | Lachnospirales | Lachnospiraceae | Blautia_A | | 665 | 731 |
| Caudovirales | | | Bacteria | Firmicutes_A | Clostridia | Lachnospirales | Lachnospiraceae | | | 667 | 688 |
| Caudovirales | | | Bacteria | Firmicutes_A | Clostridia | Lachnospirales | Lachnospiraceae | | | 667 | 688 |
| Caudovirales | Siphoviridae | | Bacteria | Firmicutes_A | Clostridia | Lachnospirales | Lachnospiraceae | | | 658 | 728 |
| Caudovirales | | | Bacteria | Firmicutes_A | Clostridia | Lachnospirales | Lachnospiraceae | | | 658 | 728 |
| Caudovirales | | | Bacteria | Firmicutes_A | Clostridia | Lachnospirales | Lachnospiraceae | | | 661 | 735 |
| Caudovirales | Siphoviridae | | Bacteria | | | | | | | 637 | 713 |
| Caudovirales | Siphoviridae | | Bacteria | Firmicutes_A | Clostridia | Peptostreptococcales | Peptostreptococcaceae | Intestinibacter | GUT_GENOME065434 | 638 | 713 |
| Caudovirales | Siphoviridae | | Bacteria | Firmicutes_A | Clostridia | Oscillospirales | Ruminococcaceae | CAG-353 | GUT_GENOME000448 | 640 | 722 |
| Caudovirales | Siphoviridae | | Bacteria | Firmicutes_A | Clostridia | | | | | 618 | 690 |
| Caudovirales | Siphoviridae | | Bacteria | Firmicutes_A | Clostridia | Oscillospirales | Ruminococcaceae | CAG-353 | GUT_GENOME000448 | 618 | 690 |
| Caudovirales | Siphoviridae | | Bacteria | Firmicutes_A | Clostridia | Peptostreptococcales | Peptostreptococcaceae | Intestinibacter | GUT_GENOME065434 | 639 | 714 |
| Caudovirales | Siphoviridae | | Bacteria | Firmicutes_A | Clostridia | | | | | 619 | 712 |
| Caudovirales | Siphoviridae | | Bacteria | Firmicutes_A | Clostridia | Oscillospirales | Ruminococcaceae | CAG-353 | GUT_GENOME000448 | 641 | 721 |
| Caudovirales | Siphoviridae | | Bacteria | Firmicutes | Bacilli | RF39 | | | | 613 | 691 |
| Caudovirales | Siphoviridae | | Bacteria | Firmicutes | Bacilli | RF39 | | | | 613 | 691 |
| Caudovirales | Siphoviridae | | Bacteria | Firmicutes | Bacilli | RF39 | | | | 614 | 692 |

TABLE 11
TAD2 IMG homologs

| | DNA | AA |
|---|---|---|
| | sequence | sequence |
| | SEQ ID | SEQ ID |
| Species | NO | NO |
| Bacillus virus SPO1 | 757 | 1046 |
| Maridesulfovibrio bastinii DSM 16055 | 758 | 1047 |
| Ruminococcus callidus ATCC 27760 | 759 | 1048 |
| Nitrosospira sp. 1 Nsp1 | 760 | 1049 |
| Escherichia coli AIEC_UM149 | 761 | 1050 |
| Myroides odoratus XSfan | 762 | 1051 |
| Epibacterium mobile 45A6 | 763 | 1052 |
| Lactiplantibacillus plantarum VBLLa 11-47 | 764 | 1053 |
| Myroides odoratus DSM 2801 | 765 | 1054 |
| Synechococcus sp. PCC 6312 | 766 | 1055 |
| Clostridium pasteurianum BC1 | 767 | 1056 |
| Bacteroides xylanisolvens NLAE-zl-P352 | 768 | 1057 |
| Bacteroides xylanisolvens NLAE-zl-P393 | 780 | 1057 |
| Bacteroides xylanisolvens NLAE-zl-P727 | 780 | 1057 |
| Bacteroides xylanisolvens NLAE-zl-P732 | 780 | 1057 |
| Cohnella panacarvi Gsoil 349, DSM 18696 | 781 | 1058 |
| Rudanella lutea DSM 19387 | 782 | 1059 |
| Bacillus sp. 105MF | 783 | 1060 |
| Thermomonas fusca DSM 15424 | 784 | 1061 |
| Aurantimonas coralicida DSM 14790 | 785 | 1062 |
| Halomonas jeotgali Hwa | 786 | 1063 |
| Sporomusa ovata DSM 2662 | 787 | 1064 |
| Clostridium cadaveris AGR2141 | 788 | 1065 |
| Oceanicola sp. S124 | 789 | 1066 |
| Ruminococcaceae bacterium LM158 | 790 | 1067 |
| Methylobacterium sp. EUR3 AL-11 | 791 | 1068 |
| Microvirgula aerodenitrificans DSM 15089 | 792 | 1069 |
| Acinetobacter tandoii DSM 14970 | 793 | 1070 |
| Megasphaera elsdenii T81 | 794 | 1071 |
| Psychrobacter phenylpyruvicus DSM 7000 | 795 | 1072 |
| Liquorilactobacillus nagelii DSM 13675 | 796 | 1073 |
| Methylomicrobium agile ATCC 35068 | 797 | 1074 |
| Methylomicrobium agile ATCC 35068 | 798 | 1075 |
| Azoarcus communis DSM 12120 | 799 | 1076 |
| Methylobacterium sp. UNCCL110 | 800 | 1077 |
| Marinomonas polaris DSM 16579 | 801 | 1078 |
| Rhizobium sp. CF258 | 802 | 1079 |
| Clostridium intestinale DSM 6191 | 803 | 1080 |
| Anaerosporobacter mobilis DSM 15930 | 804 | 1081 |
| Mameliella alba DSM 26384 | 805 | 1082 |
| Methylobacterium sp. UNCCL125 | 800 | 1077 |
| Ancylobacter rudongensis CGMCC 1.1761 | 806 | 1083 |
| Klebsiella pneumoniae 440_1540 | 807 | 1084 |
| Klebsiella pneumoniae 646_1568 | 807 | 1084 |
| Klebsiella pneumoniae 500_1420 | 807 | 1084 |
| Klebsiella pneumoniae 540_1460 | 808 | 1085 |
| Klebsiella pneumoniae 361_1301 | 807 | 1084 |
| Yersinia pseudotuberculosis B-7195 | 809 | 1086 |
| Levilactobacillus brevis ATCC 14869 | 810 | 1087 |
| Paenibacillus gorillae G1 | 811 | 1088 |
| Pasteurella multocida multocida P1062 | 812 | 1089 |
| Campylobacter concisus UNSWCS | 813 | 1090 |
| Prevotella disiens DNF00882 | 814 | 1091 |
| Campylobacter concisus UNSW2 | 815 | 1092 |
| Methylobacterium sp. UNCCL126 | 842 | 1077 |
| Novosphingobium pentaromativorans US6-1 | 816 | 1093 |
| Escherichia coli 2-474-04_S1_C1 | 817 | 1094 |
| Escherichia coli 4-203-08_S1_C1 | 818 | 1095 |
| Klebsiella pneumoniae UCI 61 | 796 | 1085 |
| Klebsiella pneumoniae BWH 47 | 796 | 1085 |
| Klebsiella pneumoniae UCI 55 | 796 | 1085 |
| Klebsiella pneumoniae MGH-73 | 796 | 1085 |
| Klebsiella pneumoniae CHS 31 | 796 | 1085 |
| Klebsiella pneumoniae UCI 59 | 795 | 1084 |
| Klebsiella pneumoniae UCI 63 | 795 | 1084 |
| Klebsiella pneumoniae MGH-79 | 796 | 1085 |
| Klebsiella pneumoniae KP4-R | 796 | 1085 |
| Bacillus thuringiensis NBIN-866 | 807 | 1096 |
| Bacillus thuringiensis sv. kurstaki HD-1 | 808 | 1097 |
| Bacillus thuringiensis sv. kurstaki HD-1 | 809 | 1098 |
| Clostridium botulinum sv. C/D BKT75002 | 810 | 1099 |
| Klebsiella pneumoniae 7699 | 795 | 1084 |
| Lactiplantibacillus paraplantarum L-ZS9 | 811 | 1100 |
| Burkholderia sp. YR281 | 812 | 1101 |
| Salipiger marinus DSM 26424 | 813 | 1102 |
| Maribius pelagius DSM 26893 | 814 | 1103 |
| bacterium YEK0313 | 815 | 1104 |
| Xenorhabdus bovienii oregonense | 816 | 1105 |
| Trichococcus collinsii DSM 14526 | 817 | 1106 |
| Klebsiella pneumoniae pneumoniae ST258-K26BO | 796 | 1085 |
| Klebsiella pneumoniae pneumoniae ST258-K28BO | 796 | 1085 |
| Clostridium jeddahense JCD | 818 | 1107 |
| Paenibacillus durus ATCC 35681 | 819 | 1108 |
| Lysinibacillus boronitolerans | 820 | 1109 |
| JCM 21713 = 10a = NBRC 103108 | | |
| Klebsiella pneumoniae pneumoniae KPNIH30 | 795 | 1084 |
| Pontibacillus litoralis JSM 072002 | 821 | 1110 |
| Bacillus cereus 03BB87 | 822 | 1111 |
| Yersinia pseudotuberculosis 4284 | 797 | 1086 |
| Klebsiella pneumoniae pneumoniae KPNIH32 | 796 | 1085 |
| Bacillus thuringiensis HD1002 | 823 | 1112 |
| Bacillus thuringiensis HD1002 | 824 | 1113 |
| Bacillus thuringiensis HD1002 | 825 | 1114 |
| Bacillus thuringiensis HD1002 | 826 | 1115 |
| Paenibacillus polymyxa NRRL B-30509 | 827 | 1116 |
| Lactiplantibacillus herbarum TCF032-E4 | 828 | 1117 |
| Yersinia pseudotuberculosis IP 32953 | 797 | 1086 |
| Lactiplantibacillus plantarum PS128 | 828 | 1117 |
| Neisseria arctica KH1503 | 829 | 1118 |
| Escherichia coli JT2 | 830 | 1094 |
| Methyloceanibacter caenitepidi Gela4 | 831 | 1119 |
| Bacillus thuringiensis sv. galleriae 4G5 | 808 | 1097 |
| Bacillus thuringiensis sv. galleriae 4G5 | 809 | 1098 |
| Bacillus thuringiensis sv. galleriae 4G5 | 808 | 1097 |
| Bacillus thuringiensis sv. galleriae 4G5 | 809 | 1098 |
| Bacillus sp. GeD10 | 832 | 1120 |
| Tenacibaculum sp. Mar_2009_124 | 833 | 1121 |
| Bacillus cereus G9241 | 822 | 1111 |
| Bacillus thuringiensis sv. Kurstaki HD 1 | 809 | 1098 |
| Bacillus thuringiensis sv. Kurstaki HD 1 | 808 | 1097 |
| Klebsiella pneumoniae pneumoniae KPNIH33 | 796 | 1085 |
| Levilactobacillus brevis DmCS_003 | 834 | 1122 |
| Levilactobacillus brevis DmCS_003 | 835 | 1123 |
| Escherichia coli G3/10 | 836 | 1124 |
| Tatumella morbirosei LMG 23360 | 837 | 1125 |
| Bacillus thuringiensis israelensis ATCC 35646 | 823 | 1112 |
| Escherichia coli ISC56 | 797 | 1085 |
| Levilactobacillus brevis WK12 | 838 | 1126 |
| Lactiplantibacillus pentosus FL0421 | 839 | 1127 |
| Lactiplantibacillus pentosus FL0421 | 840 | 1128 |
| Variovorax sp. 278MFTsu5.1 | 841 | 1129 |
| Methylobacterium sp. CL129 | 788 | 1077 |
| Paenibacillus sp. Soil724D2 | 842 | 1130 |
| Bacillus sp. Root11 | 824 | 1113 |
| Bacillus sp. Root11 | 823 | 1112 |
| Bacillus sp. Root11 | 826 | 1115 |
| Bacillus sp. Root11 | 825 | 1114 |
| Bacillus sp. Root131 | 824 | 1113 |
| Bacillus sp. Root131 | 823 | 1112 |
| Bacillus sp. Root131 | 826 | 1115 |
| Bacillus sp. Root131 | 825 | 1114 |
| Sphingobium sp. Leaf26 | 843 | 1131 |
| Pseudomonas sp. Leaf127 | 844 | 1132 |
| Comamonas thiooxydans DS1 | 845 | 1133 |
| Lactiplantibacillus plantarum Nizo2877 | 846 | 1134 |
| Klebsiella pneumoniae pneumoniae KK207/1 | 796 | 1085 |
| Bacillus thuringiensis sv. Morrisoni HD 600 | 847 | 1135 |
| Bacillus thuringiensis 147 | 824 | 1113 |
| Bacillus thuringiensis 147 | 823 | 1112 |
| Comamonas testosteroni KF712 | 848 | 1136 |
| Lacticaseibacillus paracasei NRIC 0644 | 849 | 1137 |
| Haemophilus influenzae HI1426 | 850 | 1138 |
| Bacillus thuringiensis AK47 | 809 | 1098 |
| Bacillus thuringiensis AK47 | 808 | 1097 |
| Bacillus cereus B4158 | 851 | 1139 |
| Bacillus cereus B4158 | 823 | 1112 |
| Bacillus cereus B4158 | 808 | 1097 |
| Bacillus cereus B4158 | 809 | 1098 |
| Bacillus cereus 6/27/S | 809 | 1098 |
| Bacillus cereus 6/27/S | 808 | 1097 |
| Bacillus cereus 6/27/S | 852 | 1140 |
| Bacillus cereus 6/27/S | 852 | 1140 |
| Mesorhizobium plurifarium ORS1032 | 853 | 1141 |
| Klebsiella pneumoniae MRSN 6902 | 795 | 1084 |
| Bacillus cereus B4080 | 854 | 1142 |
| Bacillus cereus RIVM BC 934 | 809 | 1098 |
| Bacillus cereus RIVM BC 934 | 808 | 1097 |
| Bacillus cereus RIVM BC 934 | 852 | 1140 |
| Bacillus cereus RIVM BC 934 | 852 | 1140 |
| Betaproteobacteria FNEB7 bin_39 | 855 | 1143 |
| Bacteroidetes FNEB6 bin_58 | 856 | 1144 |
| Bacillus thuringiensis BMB3201 | 851 | 1139 |
| Bacillus thuringiensis BMB3201 | 823 | 1112 |
| Comamonas thiooxydans KY3 | 857 | 1145 |
| Lactiplantibacillus plantarum HFC8 | 858 | 1146 |
| Lacticaseibacillus paracasei NRIC 1917 | 849 | 1137 |
| Bacillus thuringiensis YC-10 | 808 | 1097 |
| Bacillus thuringiensis YC-10 | 809 | 1098 |
| Clostridium innocuum NLAE-zl-C381 | 859 | 1147 |
| Klebsiella pneumoniae UCI81 | 796 | 1085 |
| Klebsiella pneumoniae BWH53 | 796 | 1085 |
| Klebsiella pneumoniae 50518740 | 796 | 1085 |
| Klebsiella pneumoniae S_KpVA-11 | 796 | 1085 |
| Klebsiella oxytoca CHS143 | 860 | 1148 |
| Loigolactobacillus bifermentans DSM 20003 | 861 | 1149 |
| Liquorilactobacillus nagelii DSM 13675 | 784 | 1073 |
| Klebsiella pneumoniae S_KpVA-7 | 796 | 1085 |
| Aurantimonas coralicida DSM 14790 | 773 | 1062 |
| Lactococcus lactis lactis M20 | 862 | 1150 |
| Klebsiella pneumoniae S_KpVA-13 | 796 | 1085 |
| Klebsiella pneumoniae K52-74 | 796 | 1085 |
| Klebsiella pneumoniae S_KpVA-9 | 796 | 1085 |
| Klebsiella pneumoniae S_KpVA-4 | 796 | 1085 |
| Bacillus amyloliquefaciens JRS8 | 863 | 1151 |
| Klebsiella pneumoniae S_KpVA-12 | 796 | 1085 |
| Klebsiella pneumoniae S_KpVA-5 | 796 | 1085 |
| Klebsiella pneumoniae 50675619 | 864 | 1152 |
| Klebsiella pneumoniae K52-36 | 796 | 1085 |
| Mameliella alba CGMCC 1.7290 | 793 | 1082 |
| Klebsiella pneumoniae K52-10 | 796 | 1085 |
| Klebsiella pneumoniae UCI91 | 796 | 1085 |
| Escherichia coli 165 | 865 | 1153 |
| Klebsiella pneumoniae S_KpVA-6 | 796 | 1085 |
| Acinetobacter baumannii ABBL071 | 866 | 1154 |
| Salmonella enterica enterica sv. Weltevreden | 867 | 1094 |
| 2013 2776 | | |
| Klebsiella pneumoniae 50834782 | 796 | 1085 |
| Klebsiella pneumoniae K47-25 | 796 | 1085 |
| Klebsiella pneumoniae 50509588 | 796 | 1085 |
| Klebsiella pneumoniae S_KpVA-1 | 796 | 1085 |
| Lactiplantibacillus plantarum 38 | 828 | 1117 |
| Lactiplantibacillus plantarum CMPG5300 | 868 | 1155 |
| Acinetobacter tandoii KCTC 12417 | 781 | 1070 |
| Klebsiella pneumoniae FDAARGOS_91 | 869 | 1156 |
| Klebsiella pneumoniae FDAARGOS_91 | 795 | 1084 |
| Klebsiella pneumoniae K54-05 | 796 | 1085 |
| Klebsiella pneumoniae K48-58 | 796 | 1085 |
| Klebsiella pneumoniae 50732159 | 796 | 1085 |
| Klebsiella pneumoniae K66-62 | 796 | 1085 |
| Fructilactobacillus fructivorans ATCC 27394 | 870 | 1157 |
| Klebsiella pneumoniae S_50BG | 796 | 1085 |
| Bacillus thuringiensis YWC2-8 | 808 | 1097 |
| Bacillus thuringiensis YWC2-8 | 809 | 1098 |
| Klebsiella pneumoniae pneumoniae ATCC 13883 | 796 | 1085 |
| Klebsiella pneumoniae S_103BO | 796 | 1085 |
| Mesorhizobium sp. LSHC414A00 | 871 | 1158 |
| Klebsiella pneumoniae S_12PV | 796 | 1085 |
| Klebsiella pneumoniae S_86SGR | 796 | 1085 |
| Levilactobacillus brevis DSM 20054 | 798 | 1087 |
| Klebsiella pneumoniae S_24PV | 796 | 1085 |
| Nitrosovibrio sp. Nv6 | 872 | 1159 |
| Klebsiella pneumoniae S_KpVA-10 | 796 | 1085 |
| Phaeobacter sp. S60 | 873 | 1160 |
| Klebsiella pneumoniae pneumoniae TGH8 | 796 | 1085 |
| Lactobacillus nodensis NBRC 107160 | 874 | 1161 |
| Klebsiella variicola MGH114 | 875 | 1162 |
| Klebsiella pneumoniae S_102BO | 796 | 1085 |
| Klebsiella pneumoniae S_104BO | 796 | 1085 |
| Klebsiella pneumoniae S_23PV | 796 | 1085 |
| Klebsiella pneumoniae S_30BO | 796 | 1085 |
| Klebsiella pneumoniae S_43AVR | 796 | 1085 |
| Klebsiella pneumoniae S_73RE | 796 | 1085 |
| Klebsiella pneumoniae KP-45D | 876 | 1163 |
| Klebsiella pneumoniae M098451 | 796 | 1085 |
| Companilactobacillus farciminis 8-2 | 877 | 1164 |
| Magnetospirillum sp. XM-1 | 878 | 1165 |
| Acinetobacter baumannii LAC-4 | 879 | 1166 |
| Klebsiella pneumoniae 32192 | 795 | 1084 |
| Bacillus thuringiensis sv. alesti BGSC 4C1 | 880 | 1167 |
| Sporosarcina psychrophila DSM 6497 | 88 | 1168 |
| Klebsiella pneumoniae pneumoniae TGH10 | 796 | 1085 |
| Klebsiella pneumoniae FDAARGOS_127 | 795 | 1084 |
| Klebsiella pneumoniae 38547 | 796 | 1085 |
| Lactobacillus nodensis DSM 19682 | 874 | 1161 |
| Klebsiella pneumoniae 160_1080 1134709401395 | 795 | 1084 |
| Klebsiella variicola 06-268 | 882 | 1169 |
| Klebsiella pneumoniae S_42AVR | 796 | 1085 |
| Escherichia coli AZ71 | 883 | 1170 |
| Klebsiella pneumoniae K52-43 | 796 | 1085 |
| Klebsiella pneumoniae S_75RE | 796 | 1085 |
| Stenotrophomonas maltophilia LMG 22072 | 884 | 1171 |
| Klebsiella pneumoniae K66-73 | 796 | 1085 |
| Escherichia coli AY62 | 885 | 1172 |
| Klebsiella pneumoniae S_KpVA-2 | 796 | 1085 |
| Escherichia coli 505545 | 830 | 1094 |
| Klebsiella pneumoniae S_KpVA-14 | 796 | 1085 |
| Klebsiella pneumoniae S_72RE | 796 | 1085 |
| Acinetobacter baumannii ABBL070 | 866 | 1154 |
| Acinetobacter baumannii ABBL070 | 866 | 1154 |
| Acinetobacter baumannii ABBL070 | 866 | 1154 |
| Klebsiella pneumoniae S_74RE | 796 | 1085 |
| Klebsiella pneumoniae KLP41 | 795 | 1084 |
| Rhodobacter sp. JGI CrystG Aug3-1-F17 | 886 | 1173 |
| (unscreened) | | |
| Companilactobacillus bobalius DSM 19674 | 887 | 1174 |
| Salmonella enterica enterica sv. Weltevreden | 888 | 1094 |
| 08 2437 | | |
| Klebsiella pneumoniae S_KpVA-8 | 796 | 1085 |
| Klebsiella pneumoniae S_89SGR | 796 | 1085 |
| Salmonella enterica enterica sv. Weltevreden 840K | 888 | 1094 |
| Klebsiella pneumoniae K53-64 | 796 | 1085 |
| Klebsiella pneumoniae S_87SGR | 796 | 1085 |
| Klebsiella pneumoniae S_88SGR | 796 | 1085 |
| Klebsiella pneumoniae S_KpVA-15 | 796 | 1085 |
| Klebsiella pneumoniae S_KpVA-16 | 796 | 1085 |
| Klebsiella pneumoniae S_90SGR | 796 | 1085 |
| Klebsiella pneumoniae S_71RE | 796 | 1085 |
| Klebsiella pneumoniae S_KpVA-3 | 796 | 1085 |
| Mesorhizobium sp. LNJC391B00 | 889 | 1175 |
| Escherichia coli 503130_aEPEC | 830 | 1094 |
| Liquorilactobacillus uvarum DSM 19971 | 890 | 1176 |
| Lactiplantibacillus plantarum plantarum ATCC | 846 | 1134 |
| 14917 | | |
| Lactiplantibacillus plantarum 8 RA-3 | 828 | 1117 |
| Klebsiella pneumoniae S_101BO | 796 | 1085 |
| Klebsiella pneumoniae S_11PV | 796 | 1085 |
| Klebsiella pneumoniae S_13PV | 796 | 1085 |
| Klebsiella pneumoniae S_29BO | 796 | 1085 |
| Klebsiella pneumoniae S_31AVR | 796 | 1085 |
| Klebsiella pneumoniae S_48AVR | 796 | 1085 |
| Klebsiella pneumoniae S_44AVR | 796 | 1085 |
| Klebsiella pneumoniae S_54BG | 796 | 1085 |
| Klebsiella pneumoniae S_56BG | 796 | 1085 |
| Klebsiella pneumoniae S_57BG | 796 | 1085 |
| Klebsiella pneumoniae S_58BG | 796 | 1085 |
| Klebsiella pneumoniae S_97SGR | 796 | 1085 |
| Klebsiella pneumoniae pneumoniae | 891 | 1156 |
| ST323:941530379 | | |
| Bacillus thuringiensis GOE1 | 892 | 1177 |
| Bacillus thuringiensis GOE2 | 892 | 1177 |
| Klebsiella pneumoniae S_95SGR | 876 | 1163 |
| Neorhizobium galegae bv. orientalis HAMBI 2605 | 893 | 1178 |
| Yersinia similis Y228 | 894 | 1179 |
| Alphaproteobacteria bacterium | 895 | 1180 |
| RIFCSPHIGHO2_12_FULL_63_12 | | |
| Alphaproteobacteria bacterium | 896 | 1181 |
| RIFCSPHIGHO2_12_FULL_63_12 | | |
| Escherichia coli E830 | 888 | 1094 |
| Klebsiella pneumoniae MRSN6920 | 795 | 1084 |
| Klebsiella pneumoniae MRSN8157 | 795 | 1084 |
| Klebsiella pneumoniae M097273 | 796 | 1085 |
| Klebsiella pneumoniae M098513 | 796 | 1085 |
| Klebsiella pneumoniae M098440 | 796 | 1085 |
| Klebsiella pneumoniae M098441 | 796 | 1085 |
| Klebsiella pneumoniae M098442 | 796 | 1085 |
| Klebsiella pneumoniae M104145 | 796 | 1085 |
| Bacillus glycinifermentans KJ-17 | 897 | 1182 |
| Bacillus glycinifermentans GO-13 | 897 | 1182 |
| Lysinibacillus sp. F5 | 898 | 1183 |
| Klebsiella oxytoca CAV1335 | 899 | 1184 |
| Klebsiella oxytoca CAV1099 | 899 | 1184 |
| Klebsiella pneumoniae CAV1016 | 900 | 1185 |
| Klebsiella pneumoniae KPC-3 | 796 | 1085 |
| Acinetobacter baumannii LAC4 | 879 | 1166 |
| Tardibacter chloracetimidivorans JJ-A5 | 901 | 1186 |
| Klebsiella pneumoniae KPNIH36 | 796 | 1085 |
| Klebsiella pneumoniae CAV1453 | 795 | 1084 |
| Enterobacter kobei DSM 13645 | 902 | 1187 |
| Salmonella enterica enterica sv. Newport | 903 | 1188 |
| CFSAN003890 | | |
| Secundilactobacillus paracollinoides TMW 1.1994 | 904 | 1189 |
| Levilactobacillus brevis D6 | 905 | 1190 |
| Yersinia pseudotuberculosis IP33038 | 797 | 1086 |
| Yersinia mollaretii IP22404 | 906 | 1191 |
| Yersinia kristensenii IP27925 | 907 | 1192 |
| Clostridium botulinum 211 | 908 | 1193 |
| Cupriavidus sp. USMAA2-4 | 909 | 1194 |
| Haloactinopolyspora alba DSM 45211 | 910 | 1195 |
| Actinoplanes italicus DSM 43146 | 911 | 1196 |
| Bacillus sp. JCM 19041 | 912 | 1197 |
| Lysinibacillus boronitolerans 10a | 820 | 1109 |
| Dysgonomonas alginatilytica DSM 100214 | 913 | 1198 |
| Mongoliibacter ruber DSM 27929 | 914 | 1199 |
| Psychrobacter phenylpyruvicus NBRC 102152 | 783 | 1072 |
| Novosphingobium rosa NBRC 15208 | 915 | 1200 |
| Lactiplantibacillus plantarum Lp1610 | 916 | 1201 |
| Paenibacillus sp. JCM 16163 | 917 | 1202 |
| Escherichia coli CVM N33922PS | 918 | 1203 |
| Escherichia coli CVM N33574PS | 918 | 1203 |
| Comamonas sp. 26 | 919 | 1204 |
| Nitrosovibrio sp. Nv12 | 872 | 1159 |
| Escherichia coli sv. 104 FHI19 | 920 | 1205 |
| Klebsiella pneumoniae 143500500 | 796 | 1085 |
| Klebsiella pneumoniae 143500532 | 796 | 1085 |
| Klebsiella pneumoniae 143500506 | 796 | 1085 |
| Klebsiella pneumoniae 143500534 | 796 | 1085 |
| Klebsiella pneumoniae 143500531 | 796 | 1085 |
| Klebsiella pneumoniae 143500505 | 796 | 1085 |
| Klebsiella pneumoniae 143500529 | 796 | 1085 |
| Klebsiella pneumoniae 143500510 | 796 | 1085 |
| Klebsiella pneumoniae 143500512 | 796 | 1085 |
| Klebsiella pneumoniae 143500519 | 796 | 1085 |
| Klebsiella pneumoniae 143500528 | 796 | 1085 |
| Klebsiella pneumoniae 143500525 | 796 | 1085 |
| Klebsiella pneumoniae 143500514 | 796 | 1085 |
| Klebsiella pneumoniae 143500517 | 796 | 1085 |
| Klebsiella pneumoniae 143500509 | 796 | 1085 |
| Klebsiella pneumoniae 143500537 | 796 | 1085 |
| Klebsiella pneumoniae 143500535 | 796 | 1085 |
| Klebsiella pneumoniae 143500536 | 796 | 1085 |
| Klebsiella pneumoniae 143500540 | 796 | 1085 |
| Klebsiella pneumoniae 143500541 | 796 | 1085 |
| Klebsiella pneumoniae 143500538 | 796 | 1085 |
| Klebsiella pneumoniae 143500545 | 796 | 1085 |
| Klebsiella pneumoniae 143500543 | 796 | 1085 |
| Klebsiella pneumoniae 143500542 | 796 | 1085 |
| Klebsiella pneumoniae 143500546 | 796 | 1085 |
| Klebsiella pneumoniae 143500549 | 796 | 1085 |
| Klebsiella pneumoniae 143500551 | 796 | 1085 |
| Klebsiella pneumoniae 143500550 | 796 | 1085 |
| Klebsiella pneumoniae 143500548 | 796 | 1085 |
| Klebsiella pneumoniae 143500523 | 796 | 1085 |
| Klebsiella pneumoniae 143500554 | 796 | 1085 |
| Klebsiella pneumoniae 143500555 | 796 | 1085 |
| Klebsiella pneumoniae 143500552 | 796 | 1085 |
| Klebsiella pneumoniae ST258 | 796 | 1085 |
| Klebsiella pneumoniae OC648 | 796 | 1085 |
| Methylorubrum populi CD11_7 | 921 | 1206 |
| Escherichia coli APEC O2 | 922 | 1094 |
| Lactiplantibacillus plantarum Nizo2262 | 923 | 1207 |
| Lactiplantibacillus plantarum Nizo2776 | 924 | 1208 |
| Lactiplantibacillus plantarum Nizo2855 | 846 | 1134 |
| Lactiplantibacillus plantarum Nizo3894 | 923 | 1207 |
| Lactiplantibacillus plantarum ER | 923 | 1207 |
| Lactiplantibacillus plantarum 19.1 | 828 | 1117 |
| Moraxella catarrhalis CCUG 353 | 925 | 1209 |
| Tenacibaculum dicentrarchi AY7486TD | 926 | 1210 |
| Tenacibaculum dicentrarchi AY7486TD | 926 | 1210 |
| Cellvibrionaceae bacterium 017 | 927 | 1211 |
| Morganella psychrotolerans DI-20 | 928 | 1212 |
| Burkholderia territorii RF23-BP82 | 929 | 1213 |
| Sediminispirochaeta smaragdinae DSM 11293 | 930 | 1214 |
| Sediminispirochaeta smaragdinae DSM 11293 | 931 | 1215 |

TABLE 12
TAD2 MGV homologs

| | | | | | | | | | | AA | NA |
|---|---|---|---|---|---|---|---|---|---|---|---|
| | | | | | | | | | | sequence | sequence |
| | | | | | | | | | | SEQ | SEQ |
| ictv_order | ictv_family | ictv_genus | host_domain | host_phylum | host_class | host_order | host_family | host_genus | host_species | ID NO | ID NO |
| Caudovirales | Siphoviridae | | Bacteria | Proteobacteria | Gammaproteobacteria | Enterobacterales | Enterobacteriaceae | Escherichia | GUT_GENOME144544 | 1216 | 805 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1217 | 932 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1217 | 932 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1218 | 933 |
| Caudovirales | | Triavirus | Bacteria | Firmicutes_A | Clostridia | Oscillospirales | Ruminococcaceae | Ruminococcus_C | | 1219 | 934 |
| Caudovirales | | | Bacteria | Bacteroidota | Bacteroidia | Bacteroidales | Bacteroidaceae | | | 1220 | 935 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1216 | 805 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1221 | 936 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1222 | 937 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1218 | 933 |
| Caudovirales | | Uetakevirus | Bacteria | Proteobacteria | Gammaproteobacteria | Enterobacterales | Enterobacteriaceae | Klebsiella_A | | 1223 | 938 |
| Caudovirales | Siphoviridae | | Bacteria | Bacteroidota | Bacteroidia | Bacteroidales | Bacteroidaceae | | | 1224 | 939 |
| Caudovirales | | | Bacteria | Firmicutes_A | Clostridia | Lachnospirales | Lachnospiraceae | | | 1225 | 940 |
| Caudovirales | Myoviridae | | Bacteria | | | | | | | 1226 | 941 |
| Caudovirales | Siphoviridae | | Bacteria | Firmicutes_A | Clostridia | Lachnospirales | Lachnospiraceae | Dorea | GUT_GENOME000149 | 1227 | 942 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1228 | 943 |
| Caudovirales | crAss-phage | | Bacteria | Bacteroidota | Bacteroidia | Bacteroidales | Bacteroidaceae | | | 1229 | 944 |
| Caudovirales | | | Bacteria | Bacteroidota | Bacteroidia | Bacteroidales | Tannerellaceae | Parabacteroides | GUT_GENOME143131 | 1230 | 945 |
| Caudovirales | | | Bacteria | Firmicutes_A | Clostridia | Lachnospirales | Lachnospiraceae | | | 1231 | 946 |
| Caudovirales | | | Bacteria | Proteobacteria | Gammaproteobacteria | Enterobacterales | Enterobacteriaceae | Escherichia | GUT_GENOME144544 | 1216 | 947 |
| Caudovirales | | Triavirus | Bacteria | Firmicutes_A | Clostridia | Oscillospirales | Ruminococcaceae | Ruminococcus_C | | 1232 | 948 |
| Caudovirales | | | Bacteria | Bacteroidota | Bacteroidia | Bacteroidales | Tannerellaceae | Parabacteroides | GUT_GENOME143131 | 1233 | 949 |
| Caudovirales | | | Bacteria | Firmicutes_A | Clostridia | Lachnospirales | Lachnospiraceae | Dorea | GUT_GENOME000149 | 1234 | 950 |
| Caudovirales | | | Bacteria | Bacteroidota | Bacteroidia | Bacteroidales | Tannerellaceae | Parabacteroides | GUT_GENOME143131 | 1233 | 949 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1235 | 951 |
| Caudovirales | Siphoviridae | | Bacteria | Bacteroidota | Bacteroidia | Bacteroidales | Muribaculaceae | UBA7173 | GUT_GENOME007566 | 1236 | 952 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | crAss-phage | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1233 | 949 |
| Caudovirales | | | Bacteria | Proteobacteria | Gammaproteobacteria | Enterobacterales | Enterobacteriaceae | Escherichia | GUT_GENOME144544 | 1216 | 805 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1221 | 936 |
| Caudovirales | Siphoviridae | | Bacteria | | | | | | | 1238 | 955 |
| Caudovirales | Siphoviridae | | Bacteria | Firmicutes | Bacilli | Haloplasmatales | Turicibacteraceae | Turicibacter | GUT_GENOME000677 | 1238 | 955 |
| Caudovirales | Podoviridae | Lederbergvirus | Bacteria | Proteobacteria | Gammaproteobacteria | Enterobacterales | Enterobacteriaceae | Escherichia | GUT_GENOME144544 | 1222 | 937 |
| Caudovirales | Siphoviridae | | Bacteria | Firmicutes_A | Clostridia | Lachnospirales | Lachnospiraceae | | | 1239 | 956 |
| Caudovirales | Siphoviridae | | Bacteria | Firmicutes_A | Clostridia | Lachnospirales | Lachnospiraceae | | | 1239 | 956 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | | | Bacteria | Bacteroidota | Bacteroidia | Bacteroidales | Tannerellaceae | Parabacteroides | GUT_GENOME143131 | 1233 | 949 |
| Caudovirales | | | Bacteria | Bacteroidota | Bacteroidia | Bacteroidales | Bacteroidaceae | | | 1229 | 944 |
| Caudovirales | | | Bacteria | Firmicutes_A | Clostridia | Lachnospirales | Lachnospiraceae | Clostridium_M | | 1240 | 957 |
| Caudovirales | | | Bacteria | Firmicutes_A | Clostridia | Lachnospirales | Lachnospiraceae | | | 1241 | 958 |
| Caudovirales | | | Bacteria | Proteobacteria | Gammaproteobacteria | Enterobacterales | Enterobacteriaceae | Escherichia | GUT_GENOME144544 | 1242 | 761 |
| Caudovirales | | | Bacteria | Firmicutes_A | Clostridia | Lachnospirales | Lachnospiraceae | | | 1243 | 959 |
| Caudovirales | | | Bacteria | Firmicutes_A | Clostridia | Lachnospirales | Lachnospiraceae | | | 1243 | 959 |
| Caudovirales | Siphoviridae | | Bacteria | Proteobacteria | Gammaproteobacteria | Enterobacterales | Enterobacteriaceae | Klebsiella | GUT_GENOME147598 | 1244 | 960 |
| Caudovirales | Siphoviridae | | Bacteria | Bacteroidota | Bacteroidia | Bacteroidales | Bacteroidaceae | Bacteroides | | 1224 | 939 |
| Caudovirales | Siphoviridae | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Firmicutes_A | Clostridia | Lachnospirales | Lachnospiraceae | Dorea | GUT_GENOME000149 | 1227 | 942 |
| Caudovirales | | | Bacteria | Firmicutes_A | Clostridia | Lachnospirales | Lachnospiraceae | Dorea | GUT_GENOME000149 | 1227 | 942 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Firmicutes_A | Clostridia | Lachnospirales | Lachnospiraceae | Dorea | GUT_GENOME000149 | 1245 | 961 |
| Caudovirales | | | Bacteria | Firmicutes_A | Clostridia | Lachnospirales | Lachnospiraceae | Dorea | GUT_GENOME000149 | 1246 | 962 |
| Caudovirales | | | Bacteria | Firmicutes_A | Clostridia | Lachnospirales | Lachnospiraceae | | | 1247 | 963 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Firmicutes_A | Clostridia | Lachnospirales | Lachnospiraceae | | | 1241 | 958 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1248 | 964 |
| Caudovirales | | | Bacteria | Bacteroidota | Bacteroidia | Bacteroidales | Bacteroidaceae | | | 1229 | 944 |
| Caudovirales | | | Bacteria | Bacteroidota | Bacteroidia | Bacteroidales | Bacteroidaceae | | | 1229 | 944 |
| Caudovirales | Siphoviridae | | Bacteria | Firmicutes_A | Clostridia | Lachnospirales | Lachnospiraceae | Dorea | GUT_GENOME000149 | 1249 | 965 |
| Caudovirales | | | Bacteria | Firmicutes_A | Clostridia | Lachnospirales | Lachnospiraceae | Dorea | GUT_GENOME000149 | 1234 | 950 |
| Caudovirales | | | Bacteria | Firmicutes_A | Clostridia | Lachnospirales | Lachnospiraceae | Dorea | GUT_GENOME000149 | 1234 | 950 |
| Caudovirales | Siphoviridae | | Bacteria | Firmicutes_A | Clostridia | Clostridiales | Clostridiaceae | Clostridium | GUT_GENOME036763 | 1250 | 966 |
| Caudovirales | Siphoviridae | | Bacteria | Firmicutes_A | Clostridia | Clostridiales | Clostridiaceae | Clostridium | GUT_GENOME036763 | 1250 | 966 |
| Caudovirales | | | Bacteria | Firmicutes_A | Clostridia | Lachnospirales | Lachnospiraceae | Dorea | GUT_GENOME000149 | 1227 | 942 |
| Caudovirales | | | Bacteria | Firmicutes_A | Clostridia | Lachnospirales | Lachnospiraceae | Dorea | GUT_GENOME000149 | 1227 | 942 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | crAss-phage | | Bacteria | Bacteroidota | Bacteroidia | Bacteroidales | Tannerellaceae | Parabacteroides | GUT_GENOME143131 | 1233 | 954 |
| Caudovirales | crAss-phage | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | | | | | | | | 1251 | 967 |
| Caudovirales | | | Bacteria | Bacteroidota | Bacteroidia | Bacteroidales | Tannerellaceae | Parabacteroides | GUT_GENOME143131 | 1233 | 949 |
| Caudovirales | | | Bacteria | Bacteroidota | Bacteroidia | Bacteroidales | Bacteroidaceae | Prevotella | GUT_GENOME140299 | 1252 | 968 |
| Caudovirales | | | Bacteria | Bacteroidota | Bacteroidia | Bacteroidales | Bacteroidaceae | Bacteroides | GUT_GENOME147854 | 1229 | 969 |
| Caudovirales | | | Bacteria | Bacteroidota | Bacteroidia | Bacteroidales | Tannerellaceae | Parabacteroides | GUT_GENOME143131 | 1233 | 954 |
| Caudovirales | | | Bacteria | Bacteroidota | Bacteroidia | Bacteroidales | Tannerellaceae | Parabacteroides | GUT_GENOME000192 | 1253 | 970 |
| Caudovirales | | | Bacteria | Bacteroidota | Bacteroidia | Bacteroidales | Tannerellaceae | Parabacteroides | GUT_GENOME000192 | 1253 | 970 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1256 | 973 |
| Caudovirales | | | Bacteria | Bacteroidota | Bacteroidia | Bacteroidales | Tannerellaceae | Parabacteroides | GUT_GENOME000192 | 1257 | 974 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1257 | 976 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 977 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1259 | 978 |
| Caudovirales | | | Bacteria | Bacteroidota | Bacteroidia | Bacteroidales | Tannerellaceae | Parabacteroides | GUT_GENOME000192 | 1260 | 979 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | | 1261 | 981 |
| Caudovirales | | | Bacteria | Bacteroidota | Bacteroidia | Bacteroidales | Tannerellaceae | Parabacteroides | GUT_GENOME000192 | 1262 | 982 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1264 | 984 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1265 | 986 |
| Caudovirales | | | Bacteria | Bacteroidota | Bacteroidia | Bacteroidales | Tannerellaceae | Parabacteroides | GUT_GENOME000192 | 1265 | 986 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1264 | 984 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1266 | 987 |
| Caudovirales | | | Bacteria | Bacteroidota | Bacteroidia | Bacteroidales | Tannerellaceae | Parabacteroides | GUT_GENOME000192 | 1267 | 988 |
| Caudovirales | | | Bacteria | Bacteroidota | Bacteroidia | Bacteroidales | Tannerellaceae | Parabacteroides | GUT_GENOME000192 | 1255 | 989 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 990 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1268 | 991 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 992 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1255 | 993 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1269 | 994 |
| Caudovirales | | | Bacteria | Bacteroidota | Bacteroidia | Bacteroidales | | | | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1265 | 995 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1270 | 996 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1271 | 998 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1255 | 999 |
| Caudovirales | | | Bacteria | Bacteroidota | Bacteroidia | Bacteroidales | Tannerellaceae | Parabacteroides | GUT_GENOME000192 | 1272 | 1000 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1273 | 1001 |
| Caudovirales | | | Bacteria | Bacteroidota | Bacteroidia | Bacteroidales | Tannerellaceae | Parabacteroides | GUT_GENOME000192 | 1271 | 998 |
| Caudovirales | crAss-phage | | Bacteria | Bacteroidota | Bacteroidia | Bacteroidales | Tannerellaceae | Parabacteroides | GUT_GENOME000192 | 1258 | 1002 |
| Caudovirales | | | Bacteria | Bacteroidota | Bacteroidia | Bacteroidales | Tannerellaceae | Parabacteroides | GUT_GENOME000192 | 1271 | 998 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1260 | 979 |
| Caudovirales | | | Bacteria | Bacteroidota | Bacteroidia | Bacteroidales | Tannerellaceae | Parabacteroides | | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1260 | 979 |
| Caudovirales | | | Bacteria | Bacteroidota | Bacteroidia | Bacteroidales | Tannerellaceae | Parabacteroides | GUT_GENOME000192 | 1274 | 1004 |
| Caudovirales | | | Bacteria | Bacteroidota | Bacteroidia | Bacteroidales | Tannerellaceae | Parabacteroides | GUT_GENOME000192 | 1259 | 1005 |
| Caudovirales | | | Bacteria | Bacteroidota | Bacteroidia | Bacteroidales | Tannerellaceae | Parabacteroides | GUT_GENOME000192 | 1275 | 1006 |
| Caudovirales | | | Bacteria | Bacteroidota | Bacteroidia | Bacteroidales | Tannerellaceae | Parabacteroides | GUT_GENOME000192 | 1264 | 1007 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1255 | 977 |
| Caudovirales | | | Bacteria | Bacteroidota | Bacteroidia | Bacteroidales | Tannerellaceae | Parabacteroides | | 1259 | 1008 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1276 | 1009 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1263 | 1010 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1255 | 1011 |
| Caudovirales | | | Bacteria | Bacteroidota | Bacteroidia | Bacteroidales | | | | 1262 | 1012 |
| Caudovirales | | | Bacteria | Bacteroidota | Bacteroidia | Bacteroidales | Tannerellaceae | Parabacteroides | GUT_GENOME000192 | 1277 | 1013 |
| Caudovirales | | | Bacteria | Bacteroidota | Bacteroidia | Bacteroidales | Tannerellaceae | Parabacteroides | GUT_GENOME000192 | 1277 | 1013 |
| Caudovirales | | | Bacteria | Bacteroidota | Bacteroidia | Bacteroidales | Tannerellaceae | Parabacteroides | GUT_GENOME000192 | 1259 | 1014 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1278 | 1015 |
| Caudovirales | | | Bacteria | Bacteroidota | Bacteroidia | Bacteroidales | Tannerellaceae | Parabacteroides | GUT_GENOME000192 | 1279 | 1016 |
| Caudovirales | | | Bacteria | Bacteroidota | Bacteroidia | Bacteroidales | Tannerellaceae | Parabacteroides | GUT_GENOME000192 | 1258 | 1017 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1263 | 1010 |
| Caudovirales | | | Bacteria | Bacteroidota | Bacteroidia | Bacteroidales | Tannerellaceae | Parabacteroides | GUT_GENOME000192 | 1280 | 1018 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1259 | 1005 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | | 1255 | 989 |
| Caudovirales | | | Bacteria | Bacteroidota | Bacteroidia | Bacteroidales | Tannerellaceae | Parabacteroides | GUT_GENOME000192 | 1281 | 1019 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1274 | 1004 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1282 | 1020 |
| Caudovirales | | | Bacteria | Bacteroidota | Bacteroidia | Bacteroidales | Tannerellaceae | Parabacteroides | GUT_GENOME000192 | 1282 | 1020 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1283 | 1021 |
| Caudovirales | | | Bacteria | Bacteroidota | Bacteroidia | Bacteroidales | Tannerellaceae | Parabacteroides | GUT_GENOME000192 | 1284 | 1022 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1263 | 1010 |
| Caudovirales | | | Bacteria | Bacteroidota | Bacteroidia | Bacteroidales | Tannerellaceae | Parabacteroides | GUT_GENOME000192 | 1285 | 1023 |
| Caudovirales | | | Bacteria | Bacteroidota | Bacteroidia | Bacteroidales | Tannerellaceae | Parabacteroides | GUT_GENOME000192 | 1286 | 1024 |
| Caudovirales | | | Bacteria | Bacteroidota | Bacteroidia | Bacteroidales | Tannerellaceae | Parabacteroides | GUT_GENOME000192 | 1255 | 1025 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1287 | 1026 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 993 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1264 | 1027 |
| Caudovirales | | | Bacteria | Bacteroidota | Bacteroidia | Bacteroidales | Tannerellaceae | Parabacteroides | GUT_GENOME000192 | 1280 | 1018 |
| Caudovirales | | | Bacteria | Bacteroidota | Bacteroidia | Bacteroidales | Tannerellaceae | Parabacteroides | GUT_GENOME000192 | 1260 | 1028 |
| Caudovirales | | | Bacteria | Bacteroidota | Bacteroidia | Bacteroidales | Tannerellaceae | Parabacteroides | GUT_GENOME000192 | 1256 | 1029 |
| Caudovirales | | | Bacteria | Bacteroidota | Bacteroidia | Bacteroidales | Tannerellaceae | Parabacteroides | GUT_GENOME000192 | 1263 | 1030 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | | 1258 | 975 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | | 1264 | 1007 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1284 | 1022 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1288 | 1031 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1259 | 1032 |
| Caudovirales | | | Bacteria | Bacteroidota | Bacteroidia | Bacteroidales | Tannerellaceae | Parabacteroides | GUT_GENOME000192 | 1272 | 1000 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1259 | 1032 |
| Caudovirales | | | Bacteria | Bacteroidota | Bacteroidia | Bacteroidales | Tannerellaceae | Parabacteroides | GUT_GENOME000192 | 1264 | 984 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 1033 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1263 | 1034 |
| Caudovirales | | | Bacteria | Bacteroidota | Bacteroidia | Bacteroidales | Tannerellaceae | Parabacteroides | GUT_GENOME000192 | 1270 | 996 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1281 | 1019 |
| Caudovirales | | | Bacteria | Bacteroidota | Bacteroidia | Bacteroidales | Tannerellaceae | Parabacteroides | GUT_GENOME000192 | 1289 | 1035 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | | | | 1259 | 978 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1290 | 1036 |
| Caudovirales | | | Bacteria | Bacteroidota | Bacteroidia | Bacteroidales | Tannerellaceae | Parabacteroides | GUT_GENOME000192 | 1264 | 984 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | | | | 1255 | 1037 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1265 | 1038 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1265 | 1038 |
| Caudovirales | | | Bacteria | Bacteroidota | Bacteroidia | Bacteroidales | Tannerellaceae | Parabacteroides | GUT_GENOME000192 | 1265 | 985 |
| Caudovirales | | | Bacteria | Bacteroidota | Bacteroidia | Bacteroidales | Tannerellaceae | Parabacteroides | GUT_GENOME000192 | 1291 | 1039 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1262 | 997 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1255 | 1040 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1255 | 1003 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | Bacteria | Bacteroidota | Bacteroidia | Bacteroidales | Tannerellaceae | Parabacteroides | GUT_GENOME000192 | 1263 | 983 |
| Caudovirales | | | Bacteria | Bacteroidota | Bacteroidia | Bacteroidales | Tannerellaceae | Parabacteroides | GUT_GENOME000192 | 1258 | 975 |
| Caudovirales | | | Bacteria | Bacteroidota | Bacteroidia | Bacteroidales | Tannerellaceae | Parabacteroides | GUT_GENOME000192 | 1255 | 980 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | Bacteria | Bacteroidota | Bacteroidia | Bacteroidales | Tannerellaceae | Parabacteroides | GUT_GENOME000192 | 1292 | 1041 |
| Caudovirales | | | Bacteria | Bacteroidota | Bacteroidia | Bacteroidales | Tannerellaceae | Parabacteroides | GUT_GENOME000192 | 1265 | 1042 |
| Caudovirales | | | Bacteria | Bacteroidota | Bacteroidia | Bacteroidales | Tannerellaceae | Parabacteroides | GUT_GENOME000192 | 1255 | 972 |
| Caudovirales | | | | | | | | | | 1293 | 1043 |
| Caudovirales | | | Bacteria | Bacteroidota | Bacteroidia | Bacteroidales | Tannerellaceae | Parabacteroides | GUT_GENOME000192 | 1259 | 1014 |
| Caudovirales | | | Bacteria | Bacteroidota | Bacteroidia | Bacteroidales | Tannerellaceae | Parabacteroides | GUT_GENOME000192 | 1254 | 971 |
| Caudovirales | | | | | | | | | | 1294 | 1044 |
| Caudovirales | | | | | | | | | | 1294 | 1044 |
| Caudovirales | | | Bacteria | Bacteroidota | Bacteroidia | Bacteroidales | Tannerellaceae | Parabacteroides | GUT_GENOME143131 | 1237 | 953 |
| Caudovirales | Siphoviridae | | Bacteria | Firmicutes_A | Clostridia | Lachnospirales | Lachnospiraceae | Dorea | GUT_GENOME000149 | 1295 | 1045 |

| | | | | | | | | | |
|---|---|---|---|---|---|---|---|---|---|
| Caudovirales | Bacteria | Bacteroidota | Bacteroidia | Bacteroidales | Tannerellaceae | Parabacteroides | GUT_GENOME000192 | 1255 | 972 |
| Caudovirales | Bacteria | Bacteroidota | Bacteroidia | Bacteroidales | Tannerellaceae | Parabacteroides | GUT_GENOME000192 | 1260 | 979 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods for Examples 1-4

Phage strains, isolation and cultivation—B. subtilis phages, phi3T (BGSCID 1L1), SPO (BGSCID 1L5) and SPR (BGSCID 1L56) were obtained from the Bacillus Genetic Stock Center (BGSC). Other phages used in the study were isolated from soil samples on B. subtilis BEST7003 culture as described in Doron et al¹⁹. To this end, soil samples were added to a log phase B. subtilis BEST7003 culture and incubated overnight to enrich for B. subtilis phages. The enriched sample was centrifuged and filtered through 0.2 μm filters, and the filtered supernatant was used to perform double layer plaque assays as described in Kropinski et al.⁴⁰. Single plaques that appeared after overnight incubation were picked and re-isolated 3 times, and amplified as described below. Phages were propagated by picking a single phage plaque into a liquid culture of B. subtilis BEST7003 grown at 37° C. to OD600 of 0.3 in MMB medium until culture collapse. The culture was then centrifuged for 10 minutes at 4000 rpm and the supernatant was filtered through a 0.2 μm filter to get rid of remaining bacteria and bacterial debris. Phage titer was determined using the small drop plaque assay method⁴¹. Briefly, 300 μl of overnight culture of bacteria were mixed with 25 ml MMB, 0.5% agar supplemented with 1 mM IPTG solution and poured into a 10 cm square plate followed by incubation for 1 hour at room temperature. The phage stock was tenfold serially diluted allowing 10 μl drops of diluted phage lysate to be placed on the solidified agar. After the drops have dried up, the plates were inverted and incubated at room temperature overnight, and plaques were counted to determine phage titer. Efficiency of plating (EOP) was measured by performing small drop plaque assay with the same phage lysate on control bacteria and bacteria containing the candidate anti-defense gene and defense system, and comparing the ratio of plaque formation. When individual plaques could not be counted due to small size, a faint lysis zone across the drop area was considered to be 10 plaques.

B. subtilis BEST7003 transformation with defense systems—B. subtilis BEST7003 cells (Doron et al. Science (2018) 359(6379): eaar4120) containing a heterologous Gabija, Thoeris (specifically, from Bacillus cereus MSX-D12), Hachiman, Septu, Lamassu and Shedu or DSR2 defense systems (SEQ ID NO: 740-746, respectively), integrated into the amyE locus, were generated. As a negative control, a transformant with plasmid containing GFP was used. Transformation to B. subtilis was performed using MC medium as previously described (Wilson, G. A. & Bott, K. F. J. Bacteriol. 95, 1439-1449 (1968)]. MC medium was composed of 80 mM K2HPO4, 30 mM KH2PO4, 2% Glucose, 30 mM Trisodium citrate, 22 μg/ml Ferric ammonium citrate, 0.1% Casein Hydrolysate (CAA) and 0.2% potassium glutamate. From an overnight starter of bacteria, 30 μl were diluted in 3 ml of MC medium supplemented with 30 μl 1M MgSO4. When the culture reached 0.6 OD (37° C., 200 rpm), 300 μl was transferred to a new 15 ml tube and ˜300 ng of plasmid DNA was added. The tube was incubated for another 3 hours (37° C., 200 rpm) and the entire reaction was plated on LB agar plates supplemented with 5 μg/ml Chloramphenicol and 100 μg/ml spectinomycin and incubated overnight at 30° C. Whole-genome sequencing was then applied to all transformed B. subtilis to verify system's integrity and lack of mutations.

Cloning of candidate systems into B. subtilis BEST7003 containing defense systems —The DNA of each anti-defense gene was synthesized (Genscript Corp.) or amplified from the source phage genome using KAPA HiFi HotStart ReadyMix (Roche cat #KK2601). Following, the anti-defense gene was cloned into pSG-thrC-phSpank vector. The vector contains a p15a origin of replication and ampicillin resistance for plasmid propagation in E. coli; and a thrC integration cassette with Chloramphenicol resistance for genomic integration into B. subtilis. Systems amplified from genomic DNA were cloned using NEBuilder HiFi DNA Assembly cloning kit (NEB E5520S) and were transformed to DH5q competent cells, resulting in a plasmid containing an anti-defense gene. The cloned vector was transformed into B. subtilis BEST7003 cells containing Gabija, Thoeris and Hachiman defense systems, integrated into the amyE locus, as described hereinabove.

DNA-seq—DNA was extracted from bacteria and phages using Qiagen DNeasy blood and tissue kit (Qiagen 69504). DNA libraries were constructed using the Nextera library preparation protocol as previously published⁴². All libraries were sequenced using the Illumina NextSeq500. The sequencing reads were aligned to the reference genomes of B. subtilis BEST7003 (Genbank: AP012496) and Bacillus phages.

GAD1 Knockout in phi3T lysogenic strain—Plasmid vector pJmp3 was used for generating the gene knockout plasmid. This vector was also used as a template for generating the Spectinomycin resistance gene cassette. The upstream homologous arm and downstream homologous arm of GAD1 (SEQ ID NO: 16) were amplified from phi3T phage genome by PCR using the following primers: Primers ‘AL_GAD1_A_F’ (SEQ ID NO: 747) and ‘AL_GAD1_A_R’ (SEQ ID NO: 748) were used to amplify the upstream homologous arm of GAD1 from phi3T phage genome. Primers ‘AL_GAD1_B_F’ (SEQ ID NO: 749) and ‘AL_GAD1_B_R’ (SEQ ID NO: 750) were used to amplify the downstream homologous arm of GAD1 from phi3T phage genome. Primers ‘AL-SPEC_F’ (SEQ ID NO: 751) and ‘AL_spec_R’ (SEQ ID NO: 752) were used to amplify Spectinomycin resistance gene cassette from Jmp3 plasmid. These three parts were cloned into pJmp3 backbone using NEBuilder HiFI DNA Assembly cloning kit (NEB E5520S) and transformed to DH5q competent cells. The cloned vector was transformed into phi3T lysogenic strain (BGSCID 1L1).

NAD levels measurements—Overnight cultures were diluted 1: 100 in 350 ml MMB supplemented with 1 mM IPTG and grown at 37° C. while shaking at 200 rpm for 90 minutes. The culture was then incubated at 25° C. while shaking at 200 rpm until reaching an OD600 of 0.3. At OD600 of 0.3 a sample of 50 ml for uninfected culture (time 0) was then removed. Following, SBSphiJ phage stock was added to the culture to reach MOI of 5. Flasks were incubated at 25° C. while shaking at 200 rpm for the duration of the experiment. 50 ml samples were collected at times 75, 90, 105, 120 minutes post infection. Immediately upon sample removal (including time 0), the sample tubes were placed on ice and centrifuged at 4° C. for 10 minutes to pellet the cells. The supernatant was discarded and the tube was flesh freezed and stored at −80° C. To extract the metabolites, 600 μl of 100 mM phosphate buffer, pH 8.0, supplemented with 4 mg/ml lysozyme (Sigma cat #L6876) was added to each pellet. Following, the tubes were incubated for 10 minutes at 25° C., and returned to ice. The samples were transferred to a FastPrep Lysing Matrix B in a 2 ml tube (MP Biomedicals cat #116911100) and lysed using FastPrep bead beater for 2×40 seconds at 6 m/s. Tubes were then centrifuged at 4° C. for 10 minutes at 15,000 g. The supernatant was transferred to Amicon Ultra-0.5 Centrifugal Filter Unit 3 kDa (Merck Millipore cat #UFC500396) and centrifuged for 45 minutes at 4° C. at 12,000 g. Filtrates were taken for LC-MS analysis or for detection using an enzymatic NADase activity assay.

NADase activity assay—NADase assay was performed using the ThsA enzyme as a reporter for the presence of variant-cyclic ADPr²⁸. The ThsA protein, was expressed under the control of T7 promoter together with a C-terminal Twin-Strep tag for subsequent purification. Expression was performed in LB medium supplemented with chloramphenicol (34 mg/ml) in E. coli BL21(DE3) cells. Induction was performed with 200 μM IPTG at 15° C. overnight. The culture was harvested and lysed by a cooled cell disrupter (Constant Systems) in lysis buffer composed of 20 mM Hepes 7.5, 0.3 M NaCl, 10% glycerol and 5 mM β-mercaptoethanol, 200 KU/100 ml lysozyme, 20 μg/ml DNase, 1 mM MgCl2, 1 mM phenylmethylsulfonyl fluoride (PMSF) and protease inhibitor cocktail (Millipore cat #539134, EDTA-free Protease Inhibitor Cocktail Set III). Cell debris was sedimented by centrifugation, and the lysate supernatant was incubated with washed StrepTactin XT beads (IBA cat #2-5030-025) for 1 hour at 4° C. The sedimented beads were then packed into a column connected to an FPLC allowing the lysate to pass through the column at 1 ml/min. The column was washed with 20 ml lysis buffer. The ThsA variants were eluted from the column using elution buffer containing 50 mM Biotin, 100 mM Tris 8, 150 mM NaCl, and 1 mM EDTA. Peaks containing ThsA were injected to a size exclusion (SEC) column (Superdex 200_16/60, GE Healthcare cat #28-9893-35) equilibrated with SEC buffer (20 mM HEPES pH 7.5, 200 mM NaCl, 2 mM DTT). Monomer peak was collected from the SEC column, aliquoted and frozen at −80° C. to be used for subsequent experiments. NADase reaction was performed in black 96-well half area plates (Coming cat #3694). In each reaction microwell, ThsA purified protein was added to cell lysate or 100 mM sodium phosphate buffer yielding a final 50 μl reaction volume. 5 μl of 5 mM Nicotinamide 1, N6-ethenoadenine dinucleotide (FNAD, Sigma cat #N2630) solution were added to each well immediately prior to the beginning of measurements and mixed by pipetting to reach a concentration of 500 μM in the 50 μl final volume reaction. FNAD was used as a substrate to report NADase activity of the ThsA enzyme. Plates were incubated inside a Tecan Infinite M200 plate reader at 25° C., and measurements were taken every 1 minute at 300 nm and 410 nm excitation and emission wavelengths, respectively. Reaction rate was calculated from the linear part of the initial reaction. In cases where the initial reaction rate was too high and led to saturation, the lysate was diluted 1: 1 in 100 mM phosphate buffer and the reaction re-measured.

Pulldown of ThsA, ThsB, TAD1 —ThsA and ThsB were cloned as an operon under the arabinose-inducible promoter on pBAD plasmid. The TAD1 gene (SEQ ID NO: 5) with a C-terminal His-tag was cloned into plasmid pBbA6c⁴³. Both plasmids were co-transformed into E. coli MG1655 strain. Overnight cultures containing the pBAD-ThsA-ThsB and pBbA6c-TAD1-His constructs were diluted 1: 100 in 100 ml MMB and grown at 25° C. while shaking at 200 rpm. 1 mM IPTG and 0.2% arabinose were added at OD600 of 0.3 followed by cell harvesting 3 hours later by centrifugation at 4000 rpm, 4° C. Pellets were stored at −80° C. and then lysed with 250 μl 50 mM NaPhosphate buffer pH 8.0, 0.1 M NaCl and 1 mg/ml lysozyme (Sigma cat #L6876). Pellets resuspended with lysozyme were shaken for 10 minutes at 25° C. After 10 minutes, 750 μl of NTA-washing buffer containing Phosphate buffer 20 mM pH 7.4, 0.5M NaCl, 20 mM imidazole and 0.05% Tween 20. These samples were transferred to a FastPrep Lysing Matrix B in a 2 ml tube (MP Biomedicals cat #116911100) and lysed using FastPrep bead beater for 2×40 seconds at 6 m/s. Tubes were then centrifuged at 4° C. for 10 minutes at 15,000 g. The supernatant was then applied to magnetic Ni-NTA-magnetic beads (Qiagen 36113) and washed twice with 0.5 ml NTA-washing buffer, followed by elution with NTA-washing buffer containing 500 mM imidazole. Protein samples containing TAD1-His co-expressed with ThsA and ThsB were compared to TAD1-His alone or ThsA/ThsB without TAD-His by running on a Bolt™ 4 to 12%, Bis-Tris (Thermofisher NWO4122BOX).

Construction of dCAS9 and gRNA cassette for integration to Bacillus Subtilis thrC site —dCAS9 from Streptococcus pyogenes together with its xyl promotor and xylR were amplified from plasmid pJMP1 (addgene plasmid #79873) and the gRNA scaffold with spacer and promoter were amplified from pJMP3 (addgene plasmid #79875). Both were cloned into the pSG-thrC_phSpank_sfGFP vector. New spacers were inserted by using the overlap of primers used for NEBuilder HiFi DNA Assembly (NEB, cat #E2621). Shuttle vectors were propagated in E. coli DH5a using a p15a origin of replication with 100 μg/ml ampicillin selection. Plasmids were miniprepped from E. coli DH5a prior to transformation into the appropriate B. subtilis BEST7003 strains. The gRNA sequence that was used to target TAD1 is ‘GGAACCACTACGAAATGAT’ (SEQ ID NO: 754). The gRNA sequence that was used to target HAD1 is ‘GCTTGCTAGGATTAGTGTCC’ (SEQ ID NO: 755). The gRNA sequence that was used as the control (doesn't target TAD1 or HAD1) is ‘ctatgattgatttttttagc’ (SEQ ID NO: 756).

LC-MS analysis of v-cADPR—Sample analysis was carried out by MS-Omics as follows. The analysis was carried out using a Thermo Scientific Vanquish LC coupled to Thermo Q Exactive HF MS. An electrospray ionization interface was used as ionization source. Analysis was performed in positive ionization mode with an m/z range of 200-1000. The UPLC was performed using a slightly modified version of the protocol described Hsiao et al. 2018 (DOI 10.1021/acs.analchem.8b02100). Peak areas were extracted using Compound Discoverer 3.1 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

Genome assembly and open reading frames (ORFs) prediction—First, adapter sequences were removed from the reads of each phage genome using Cutadapt version 2.844 with the option -q 5 (quality threshold 5). The trimmed reads from each phage genome were assembled into scaffolds using SPAdes genome assembler version 3.14.045, using the -careful flag. Each assembled genome was analyzed with Prodigal version 2.6.3⁴⁶ (default parameters) to predict ORFs.

Anti-defense candidate prediction—To find candidate anti-defense genes, the protein sequences from all the phages in each phage family were clustered into groups of homologs using the cluster module in MMSeqs2 release 12-113e3⁴⁷, with the parameters -e 10, -c 0.8, -s 8, --min-seq-id 0.3 and the flag -single-step-clustering. Overall, 540 unique clusters in the SpBeta-like group, and 320 unique clusters in the SBSphiJ-like group were obtained. Next, each cluster was mapped to a list of phages that have a member of that cluster in their genome. For each defense system, anti-defense candidates were defined as clusters that have a representation in all the phages that overcome the defense system and are absent from all the phages that are blocked by the defense system. One member was chosen from each cluster as a candidate anti-defense gene for further experimental testing. In the case of the Hachiman defense system, where 9 anti-defense candidates were found, genes that are similar to housekeeping genes were neglected, while 3 candidates that are not similar to any gene with a known function were chosen for further analysis.

Identification of anti-defense homologs —All homologs were Identified by searching the anti-defense protein sequences in the integrated microbial genomes (IMG)³⁸ and the metagenomic gut virus (MGV)³⁹ databases. Homologs of GAD1 and HAD1 in the IMG database were found by using the blast option in the IMG web server (GAD1 homologs were searched using the default parameters, while HAD1 homologs were searched using an e-value of 10), and additional homologs were added manually by using the “top IMG homolog hits” option on IMG. Homologs of TAD1 were found by searching TAD1 against 38,000 genomes that were downloaded from the IMG database in October 2017, using the “search” option of MMseqs release 12-113e3 with default parameters. Homologs in the MGV databases were found for GAD1 and TAD1 using the “search” option of MMseqs release 12-113e3 with default parameters. HAD1 homologs were not found in the MGV database. The sequences were filtered for redundancy, keeping one representative for each unique sequence. Overall, 255/63/16 unique homologs for TAD1/GAD1/HAD1, respectively, were identified, see Tables 6-10 hereinabove.

Phylogenetic trees constructions and visualization—For each family of anti-defense proteins, the unique sequences were aligned using MAFFT version 7.402⁴⁸ with default parameters. The trees were constructed using IQ-TREE version 1.6.549 with the -m LG parameter. The online tool iTOL24 (v.5)⁵⁰ was used for tree visualization. Phage family annotations are based on the prediction in the MGV database. The host phyla annotations are based on the prediction in the MGV database, or the lysogen's taxonomy. Gabija and Thoeris defense systems were found in the bacterial genomes by searching for the respective pfam/COG annotations (only cases where the two genes of Gabija/Thoeris are found next to each other on the genome were collected).

Knock-in of TAD1 into phage SBSphiJ—The DNA sequence of TAD1, together with its upstream intergenic region, was amplified from the genome of phage SBSphiJ7 using KAPA HiFi HotStart ReadyMix (Roche, no. KK2601) with the primer pair CAACTGAGTAAATAAATAGAGCCTAGTGTAACGAC and CTTTGCCAAGTGTTTTCCCTCCA (SEQ ID Nos: 1303-1304). The upstream and downstream genomic arms (±1.2 kbp) for the integration site of the TAD1 insert within the SBSphiJ genome were amplified from the genome of phage SBSphiJ using the primer pair ACTCTTGTTAACTCTAGAGCTATGTCATTCTTAGACATTGTAAACCAAGAAGCAG and GGCTCTATTTATTTACTCAGTTGGCAAGTCTCC (SEQ ID Nos: 1305-1306) and the primer pair GGAAAACACTTGGCAAAGAAGAAAAAACAGAATAATGTATCC and TAGCGAAAAATCCTTTTCTTTCTTACCCTTCTCCATCAGTGTTCAATAAATCATC (SEQ ID Nos: 1307-1308), respectively. The three fragments were cloned into the pSG-thrC-Phspank backbone using the NEBuilder HiFI DNA Assembly cloning kit (NEB, no. E5520S) and transformed to DH5α-competent cells. The cloned vector was subsequently transformed into the thrC site of B. subtilis BEST7003. The TAD1-containing B. subtilis BEST7003 strain was then infected with phage SBSphiJ with an MOI of 0.1 and cell lysate was collected. The lysate was used to infect a Thoeris-containing B. subtilis culture in two consecutive rounds with an MOI of 2. Several plaques were collected and screened using PCR for TAD1-containing phages. A TAD1-contaning phage was purified three times on B. subtilis BEST7003. Purified phage was verified again for the presence of TAD1 using PCR amplification. Whole-genome sequencing was then applied to the phage to verify the integrity of the TAD1 knock-in.

### Materials and Methods for Example 5

Bacterial strains and phages—E. coli strain MG1655 (ATCC 47076) was grown in MMB (LB+0.1 mM MnCl2÷5 mM MgCl2, with or without 0.5% agar) at 37° C. or room temperature (RT). Whenever applicable, media were supplemented with ampicillin (100 μg/ml), to ensure the maintenance of plasmids. Infection was performed in MMB media at 37° C. or RT as detailed in each section. B. subtilis strain BEST7003 was grown in MMB (LB+0.1 mM MnCl2+5 mM MgCl2, with or without 0.5% agar) at 37° C. or room temperature (RT). Whenever applicable, media were supplemented with spectinomycin (100 jig/ml) and chloramphenicol (5 μg/ml), to ensure selection of transformed and integrated cells. Infection was performed in MMB media at 37° C. or RT as detailed in each section. Plasmid and strain construction —The defense system DSR2 was synthesized by Genscript Corp. and cloned into the pSG1 plasmid [Doron, S. et al. Science 359, eaar4120 (2018)] with its native promoter. DSR2(N133A) and DSR2(H171A) mutants of the DSR2 gene were constructed using Q5 Site-directed Mutagenesis kit (NEB). DSAD1 and tail-tube were amplified from phage genomic DNA and cloned into the pSG-thrC_phSpank_sfGFP vector. Inducible DSR2, and DSR2(H171A) were amplified and cloned into the plasmid pBbA6c-RFP (Addgene, cat. #35290). Inducible tail-tube, tail-tube with C-terminal twin streptavidin, and DSAD1 with C-terminal twin streptavidin were amplified and cloned into the plasmid pbBS8k-RFP (Addgene, cat. #35323).

Bacillus transformation—Transformation to B. subtilis BEST7003 was performed using MC medium as previously described (Wilson, G. A. & Bott, K. F. J. Bacteriol. 95, 1439-49 (1968)]. MC medium was composed of 80 mM K2-IP04, 30 mM KH2PO4, 2% glucose, 30 mM trisodium citrate, 22 Vg/ml ferric ammonium citrate, 0.1% casein hydrolysate (CAA), 0.2% potassium glutamate. From an overnight starter of bacteria, 10 μl were diluted in 1 ml of MC medium supplemented with 10 μl 1M MgSO4. After 3 hours of incubation (37° C., 200 rpm), 300 μl of the culture was transferred to a new 15 ml tube and ˜200 ng of plasmid DNA was added. The tube was incubated for another 3 hours (37° C., 200 rpm), and the entire reaction was plated on Lysogeny Broth (LB) agar plates supplemented with 5 μg/ml chloramphenicol or 100 μg/ml spectinomycin and incubated overnight at 30° C. Plaque assays—Phages were propagated by picking a single phage plaque into a liquid culture of B. subtilis BEST7003 or E. coli MG1655 grown at 37° C. to OD₆₀₀ 0.3 in MMB medium until culture collapse. The culture was then centrifuged for 10 minutes at 4000 r.p.m and the supernatant was filtered through a 0.2 m filter to get rid of remaining bacteria and bacterial debris. Lysate titer was determined using the small drop plaque assay method as described [Mazzocco, A., Waddell, T. E., Lingohr, E. & Johnson, R. P. Methods in molecular biology (Clifton, N.J.) 501, 81-5 (2009)]. Plaque assays were performed as previously described ([Doron, S. et al. Science 359, eaar4120 (2018); Mazzocco, A., Waddell, T. E., Lingohr, E. & Johnson, R. P. Methods in molecular biology (Clifton, N.J.) 501, 81-5 (2009)]. Bacteria containing defense system and control bacteria with no system were grown overnight at 37° C. Then 300 μl of the bacterial culture was mixed with 30 ml melted MMB 0.5% agar, poured on 10 cm square plates, and let to dry for 1 hour at room temperature. For cells that contained inducible constructs, the inducers were added to the agar before plates were poured. 10-fold serial dilutions in MMB were performed for each of the tested phages and 10 μl drops were put on the bacterial layer. After the drops had dried up, the plates were inverted and incubated at room temperature or 37° C. overnight. Plaque forming units (PFUs) were determined by counting the derived plaques after overnight incubation and lysate titer was determined by calculating PFUs per ml. When no individual plaques could not be identified, a faint lysis zone across the drop area was considered to be 10 plaques.

Phage-infection dynamics in liquid medium—Non-induced overnight cultures of bacteria containing defense system and bacteria with no system (negative control) were diluted 1 : 100 in MMB medium supplemented with appropriate antibiotics and incubated at 37° C. while shaking at 200 rpm until early log phase (OD600=0.3). 180 μl of the diluted culture were transferred into wells in a 96-well plate containing 20 μl of phage lysate for a final MOI of 4 and 0.04, or, 20 μl of MMB for uninfected control. Infections were performed in triplicates from overnight cultures prepared from separate colonies. Plates were incubated at 30° C. or 37° C. with shaking in a TECAN Infinite200 plate reader and OD600 was measurement was taken every 10 minutes.

Liquid medium growth toxicity assay—Non-induced E. coli with DSR2 and tail tube, and RFP and DSR2 (negative control), were grown in 1% glucose overnight. Cells were diluted 1: 100 in 3 ml of mmb and grown at 37° C. to an OD of 0.3 before being induced. Uninduced cells and cells induced at 0.2% arabinose and 1 mM IPTG, were transferred into a 96-well plate. Plates were incubated at 37° C. with shaking in a TECAN Infinite200 plate reader and a OD₆₀₀ measurement was taken every 10 minutes.

Cell lysate preparation for LC-MS—Overnight cultures of Bacteria containing defense system and Bacteria with no system (negative control) were diluted 1: 100 in 250 ml MMB and incubated at room temperature or 37° C. with shaking (200 rpm) until reaching OD₆₀₀ of 0.3. For cells that contained inducible constructs, the inducers were added at an OD₆₀₀ of 0.1. A sample of 50 ml of uninfected culture (time 0) was then removed, and phage stock was added to the culture to reach MOI of 5-10. Flasks were incubated at 30° C. or 37° C. with shaking (200 rpm) for the duration of the experiment. 50 ml samples were collected at various time points post infection. Immediately upon sample removal the sample tube was placed in ice, centrifuged at 4° C. for 5 minutes to pellet the cells. The supernatant was discarded and the tube was frozen at −80° C. To extract the metabolites, 600 μl of 100 mM phosphate buffer at pH 8, supplemented with 4 mg/ml lysozyme, was added to each pellet. Tubes were then incubated for 5 minutes at 37° C., and returned to ice. Thawed sample was transferred to a FastPrep Lysing Matrix 2 ml tube (MP Biomedicals cat #116911100) and lysed using FastPrep bead beater for 40 seconds at 6 m/s. Tubes were then centrifuged at 4° C. for 15 minutes at 15,000 g. Supernatant was transferred to Amicon Ultra-0.5 Centrifugal Filter Unit 3 KDa (Merck Millipore cat #UFC500396) and centrifuged for 45 minutes at 4° C. at 12,000 g. Filtrate was taken and used for LC-MS analysis.

Quantification of NAD⁺ and ADPR by HPLC-MS—Cell lysates were prepared as described above and analyzed by LC-MS/MS. Quantification of nucleotides was carried out using an Acquity I-class UPLC system coupled to Xevo TQ-S triple quadrupole mass spectrometer (both Waters, US). The UPLC was performed using an Atlantis Premier BEH C18 AX column with the dimension of 2.1×100 mm and particle size of 1.7 μm (Waters). Mobile phase A was 20 mM ammonium formate at pH 3 and acetonitrile was mobile phase B. The flow rate was kept at 300 μl/min consisting of a 2 minutes hold at 2% B, followed by linear gradient increase to 100% B during 5 minutes. The column temperature was set at 25° C. and an injection volume of 1 μl. An electrospray ionization interface was used as ionization source. Analysis was performed in positive ionization mode. Metabolites were detected using multiple-reaction monitoring, using argon as the collision gas. Quantification was made using standard curve in 0-1 mM concentration range. NAD+(Sigma) and ADPR (Sigma) were added to standards and samples as internal standard (0.5 μM). TargetLynx (Waters) was used for data analysis.

Pulldown assays of the DSR2-DSAD1 and DSR2-tail tube complex—Non-induced overnight cultures of E. coli with DSR2(H171A), DSAD1 with C-terminal Streptavidin tag, tail-tube with C-terminal Streptavidin tag, DSR2(H171A) and DSAD1 with C-terminal Streptavidin tag, and DSR2(H171A) and tail-tube with C-terminal Streptavidin tag were diluted 1: 100 in 50 ml of mmb and grown at 37° C. to an OD of 0.3. Cells were induced with 0.2% arabinose and 1 mM IPTG and continued to grow to an OD of 0.9 at 37° C. Cells were centrifuged at 4000 rpm for 10 minutes. Supernatant was discarded and pellets were frozen in −80° C. To pulldown the proteins, 1 ml of Strep wash buffer (IBA cat #2-1003-100) supplemented with 4 mg/ml lysozyme was added to each pellet and incubated for 10 minutes at 37° C. with shaking until thawed and resuspended. Tubes were then transferred to ice, and the resuspended cells transferred to a FastPrep Lysing Matrix B in 2 ml tube (MP Biomedicals cat #116911100). Samples were lysed using FastPrep bead beater for 40 seconds at 6 m/s. Tubes were centrifuged for 15 minutes at 15,000 g. Per each pellet, 30 μl of MagStrep “Type 3” XT beads (IBA cat #2-4090-002) were washed twice in 300 μl wash buffer, and the lysed cell supernatant was mixed with the beads and incubated for 30-60 minutes, rotating in 4° C. The beads were then pelleted on a magnet, washed twice with wash buffer, and purified protein was eluted from the beads in 10 μl of BXT elution buffer (IBA cat #2-1042-025). 30 μl of samples were mixed with 10 μl of 4× Bolt™ LDS Sample Buffer (ThermoFisher cat #B0008) and a final concentration of 1 mM of DTT. Samples were incubated at 75° C. for 5 minutes. Samples were loaded to a NuPAGE™ 4 to 12%, Bis-Tris, 1.0 mm, Mini Protein Gel, 12-well (ThermoFisher cat #NP0322PK2) in 20× Bolt™ MES SDS Running Buffer (ThermoFisher cat #B0002) and run at 160V. Gels were shaken with InstantBlue® Coomassie Protein Stain (ISB1L) (ab119211) for 1 hour, followed by another hour in water.

Phage coinfection and hybrid isolation—50 μl overnight culture of B. subtilis containing pVip and DSR2 was mixed with 50 μl of SPR and phi3T or SPR and SPbeta at a titer of 10⁷ pfu/ml. The Bacteria and phages were left to rest at RT for 10 minutes before being mixed with 5 ml of warm MMB 0.3% agar and poured over a low plate of MMB 0.5% agar. Plates were left overnight at RT before being inspected for plaques. Single plaques were picked into 100 μl phage buffer. To test the recombinant phages progeny for the ability to overcome pVip and dsr2 defense, the small drop plaque assay was used [Mazzocco, A., Waddell, T. E., Lingohr, E. & Johnson, R. P. Methods in molecular biology (Clifton, N.J.) 501, 81-5 (2009)]. 300 μl B. subtilis with pVip and DSR2 or negative control (No system) were mixed with 30 mL melted 0.5% and let to dry for 1 hour at room temperature. 10-fold serial dilutions in phage buffer was performed for the recombinant phages and 10 μl drops were put on the bacterial layer. The plates were incubated overnight at room temperature. Efficiency of plating (EOP) was measured and compared between the defense and NC strain.

Sequencing and assembly of phage hybrids—High titer phage lysates (>10⁷ pfu/ml) of the ancestor and isolated phage hybrids were used for DNA extraction. 500 ml of the phage lysate was treated with DNase-I (Merck cat #11284932001) added to a final concentration of 20 mg/ml and incubated at 37° C. for 1 hour to remove bacterial DNA. DNA was extracted using the QIAGEN DNeasy blood and tissue kit (cat #69504) starting from the Proteinase-K treatment step to lyse the phages. Libraries were prepared for Illumina sequencing using a modified Nextera protocol as previously described (Baym M, Krvazhimskiy S, Lieberman TD, Chung Ul, Desai MM, Kishony R (2015) Inexpensive Multiplexed Library Preparation for Megabase-Sized Genomes. PLoS ONE 10(5): e0128036). Reads were de novo assembled using Spades3.14.0.

Hybridphage alignment—Hybrid phage genomes were aligned using SnapGene Version 5.3.2. Each hybrid genome was aligned to phage SPR and areas that did not align were aligned against the other phages in the coinfection experiment in order to verify their origin and gene content.

### Materials and Methods for Example 6

Phage strains, isolation and cultivation—phage SPO1 was obtained from the Bacillus Genetic Stock Center (BGSC). Phages SPO1L1, SPO1L2, SPO1L3, SPO1L4 and SPO1L5 were isolated and cultivated as described in the Materials and Methods for Examples 1-4 hereinabove.

B. subtilis BEST7003 transformation with Thoeris and Tad2 —Transformations of Thoeris defense systems from Bacillus cereus MSX-D12 (SEQ ID NO: 741) and Bacillus amyloliquefaciens Y2 (SEQ ID NO: 1300) and the TAD2 anti Thoeris gene and its homologs (SEQ ID Nos: 757-762) into B. subtilis BEST7003 cells were performed as described in the Materials and Methods for Examples 1-4 hereinabove.

Construction of dCAS9 and gRNA cassette for integration to Bacillus Subtilis thrC site —Construction of the dCAS9 and gRNA cassette for integration to B. Subtilis thrC site was performed as described in the materials and methods for examples 1-4 hereinabove with the following modifications. The gRNA sequence that was used to target TAD2 is ‘AAGATGATGTTCCCAAACAC’ (SEQ ID NO: 1309). The gRNA sequence that was used as the control (doesn't target TAD2) is ‘GGAACCACTACGAAATGAT’ (SEQ ID NO: 754).

Open reading frames (ORFs) prediction and anti-thoeris candidate prediction—ORFs were predicted in each phage genome as described in the Materials and Methods for Examples 1-4 hereinabove. The anti Thoeris gene (Tad2) was predicted by comparing the gene content of resistant and sensitive phages to the Thoeris defense system from Bacillus cereus MSX-D12, as described in the Materials and Methods for Examples 1-4 hereinabove.

Identification of TAD2 homologs and Phylogenetic trees constructions and visualization—All TAD2 homologs were identified by searching the integrated microbial genomes (IMG)³⁸ and the metagenomic gut virus (MGV)³⁹ databases as described for Tad1 in the Materials and Methods for Examples 1-4 hereinabove. The TAD2 phylogenetic tree was constructed and visualized as described in the Materials and Methods for Examples 1-4 hereinabove.

NADase activity assay —NADase activity assay was performed as described in the Materials and Methods for Examples 1-4 hereinabove with the following modification. Instead of incubating purified ThsA with infected cell lysates, ThsA was incubated with 900 nM of 1″-3′gcADPR (the ThsB-derived signaling molecule) that was preincubated with 24 uM of purified TAD2 for 10 minutes, followed by an additional incubation of 5 minutes at either 25° C. or 95° C.

### Example 1

Identification of Phage Genes that Inhibit Bacterial Defense Systems

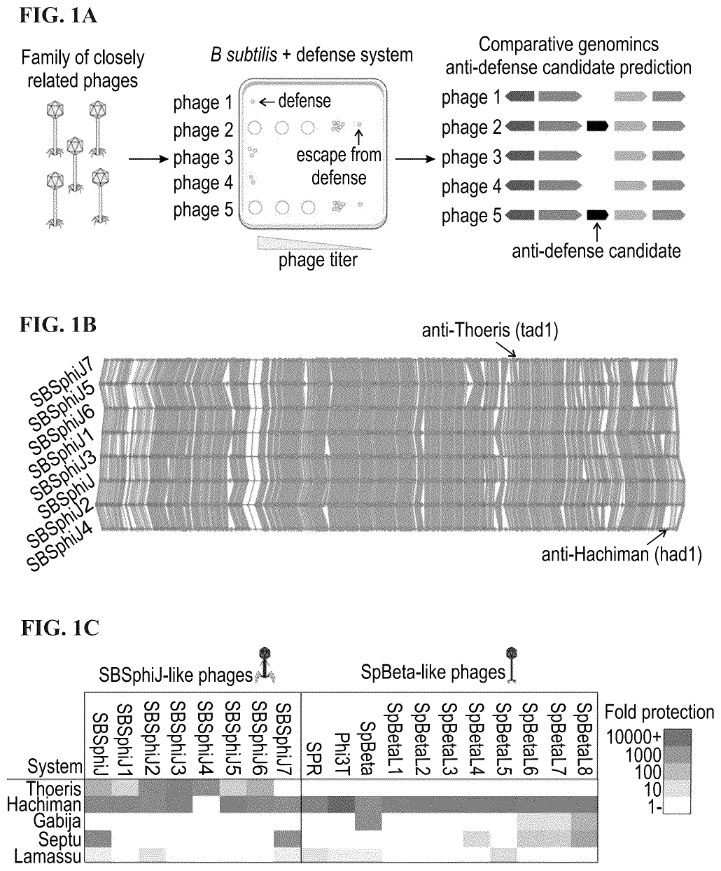

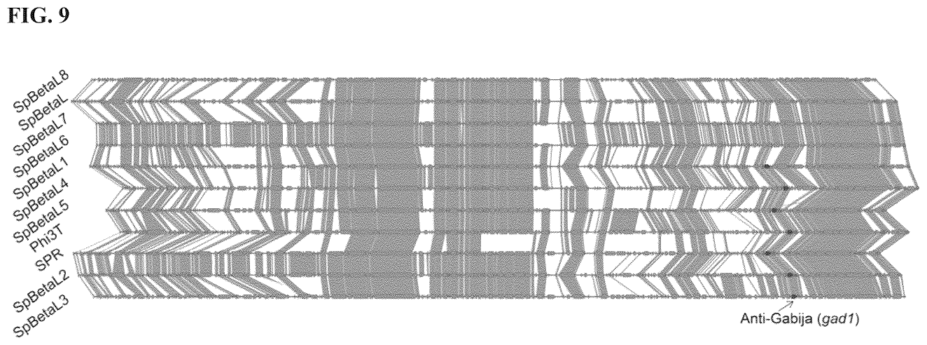

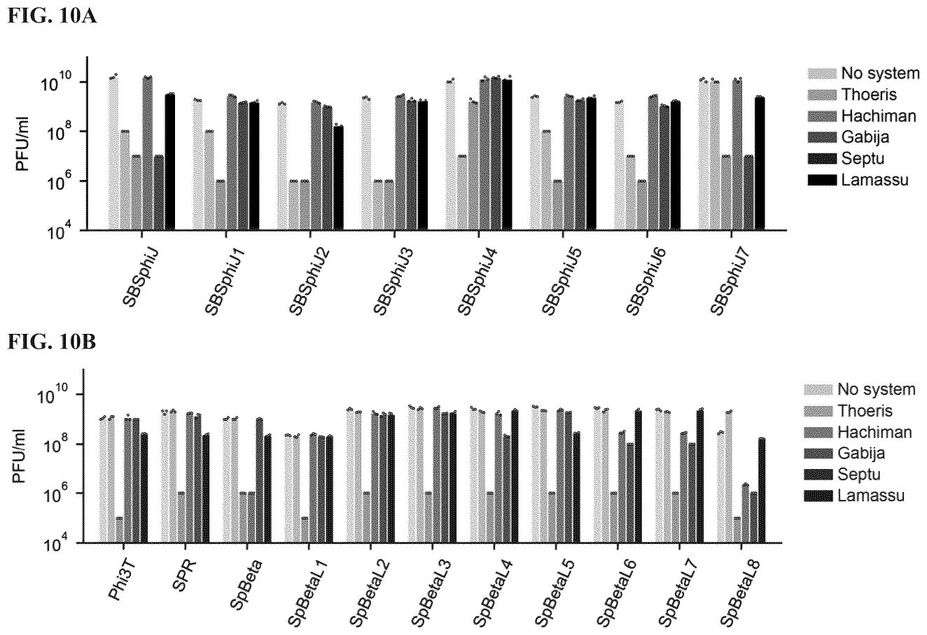

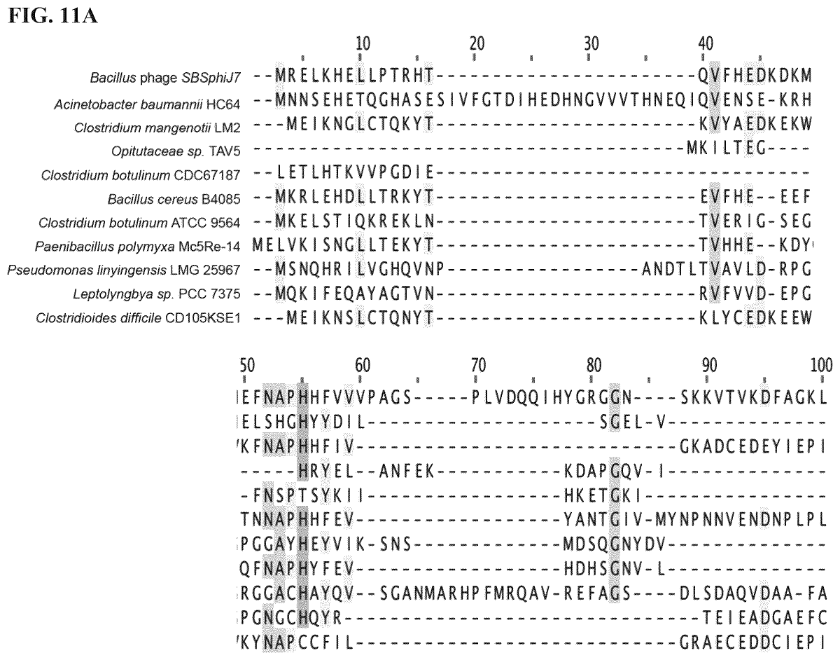

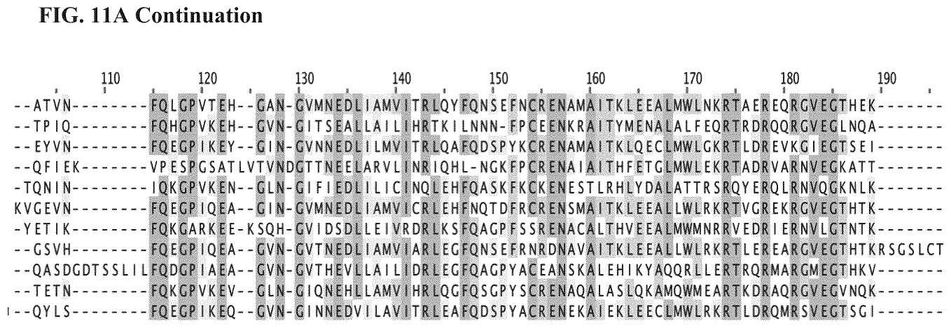

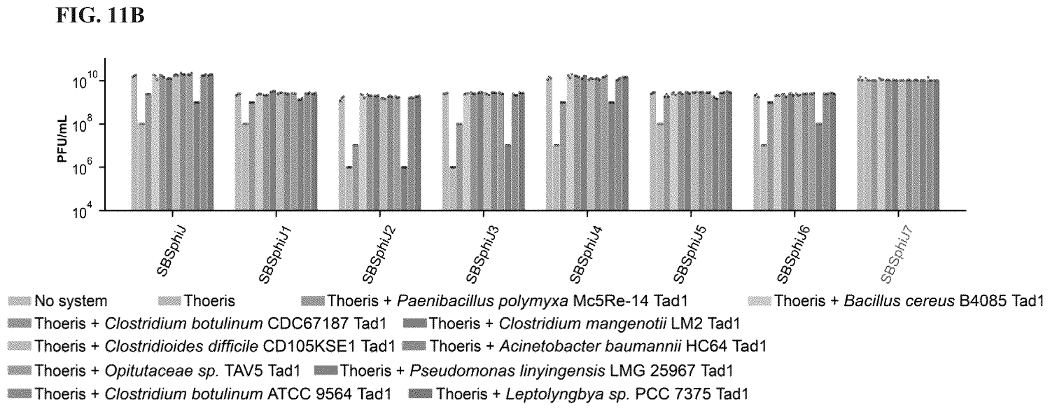

The present inventors set out to find whether some Bacillus phages can directly inhibit previously identified and described defense systems¹⁹ that protect Bacillus species from phage infection. Multiple studies demonstrated that anti-CRISPR genes are sporadically present in closely related phages, and that the presence or absence of these genes directly affects the phage sensitivity to CRISPR-Cas³¹⁻³³ Hence, it was hypothesized that genes inhibiting the new defense systems would show a similar sporadic distribution among phages, and that such genes could be predicted by examining the defense profiles and gene content of closely-related phages (FIG. 1A). To this end, two groups of closely-related phages that infect Bacillus subtilis were isolated and analyzed. One group included eight phages similar to phage SBSphiJ, a lytic Myoviridae phage with a ˜150 kb-long genome¹⁹. Between 89% and 100% of the genome was alignable when comparing pairs of phages from this group, with high (90%-100%) sequence identity in alignable regions (FIG. 1B; Tables 1-2 hereinbelow). Furthermore, eight phages were isolated from the SpBeta group, which also includes the previously isolated phages SpBeta, phi3T and SPR³⁴⁻³⁶. These are temperate Siphoviridae with ˜130 kb genomes, with 43%-96% alignable genomes when comparing phage pairs (FIG. 9, Tables 3-4 hereinbelow). Each phage had, on average, 4.5 (SBSphiJ) or 16.9 (SpBeta) genes that were not found in the genomes of other phages in the same group (data not shown).

Following, these two groups of phages were used to infect strains of Bacillus subtilis that heterologously expressed each of the defense systems described in Doron et al¹⁹, namely Thoeris (specifically, from Bacillus cereus MSX-D12), Hachiman, Gabija, Septu, Lamassu and Shedu. Five of these systems protected against at least one of the phages tested (FIG. 1C). However, phages from the same group displayed remarkably different properties when infecting defense-system-containing bacteria. For example, the Thoeris defense system protected against all phages of the SBSphiJ group except for phage SBSphiJ7, and the Hachiman defense system protected against all phages of the SBSphiJ group except for phage SBSphij4 (FIG. 1C).

To identify phage genes that may explain the differential defense phenotype, the gene content in groups of phages that overcame each defense system were analyzed, and compared to the gene content in phages that were blocked by the system. Genes common to phages that overcame the defense system, which were not found in any of the phages that were blocked by defense system, were considered as candidate anti-defense genes and were further examined experimentally, as described hereinbelow.

### Example 2

### Tad1 as an Anti-Thoeris Gene

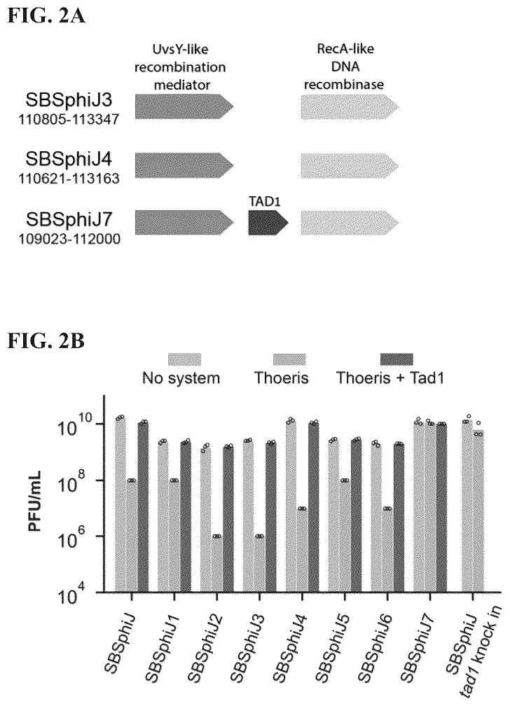

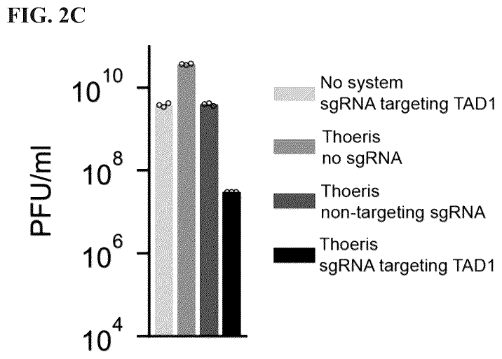

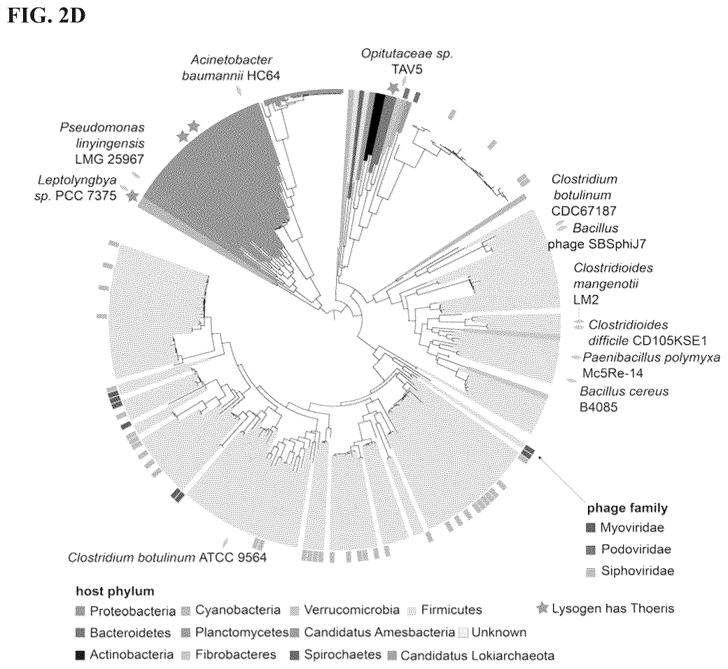

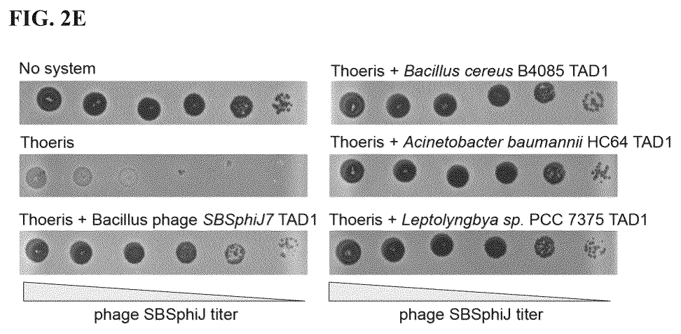

Thoeris (also known as “SIR2-TIR system”) is a common defense system found in about 4% of sequenced bacterial and archaeal genomes¹⁹. This defense system includes one or more genes with a Toll/interleukin-1 receptor (TIR) domain, which serve as sensors for phage infection²⁸. Once triggered by phage, Thoeris TIR proteins generate a signaling molecule that was shown to be an isomer of cyclic ADP-ribose (cADPR). This molecule binds a second Thoeris protein called ThsA, which becomes activated. In some embodiments, ThsA depletes the cell of nicotinamide adenine dinucleotide (NAD⁺) when active, thus causing abortive infection²⁸. In other embodiments, ThsA is a membrane-spanning effector that depolarizes the membrane when it becomes active. It was found that phage SBSphiJ7, which overcomes Thoeris defense (FIG. 1C), encodes six unique genes that did not appear in any of the phages blocked by Thoeris (Table 5 hereinbelow). Each of these six genes was cloned, under the control of an inducible promoter, into B. subtilis cells that also express the Thoeris system from Bacillus cereus MSX-D12. One of these genes, referred to herein as “TAD1 (Thoeris Anti Defense 1)” robustly inhibited the activity of the Thoeris defense system, as phages that were normally blocked by Thoeris were able to infect Thoeris-expressing cells if they also expressed TAD1 (FIGS. 2A-B). Further, silencing the expression of TAD1 in SBSphiJ7 using dCAS9³⁷ caused SBSphiJ7 to be blocked by Thoeris, verifying that TAD1 is the gene responsible for the Thoeris inhibiting phenotype of SBSphiJ7 (FIG. 2C). Moreover, knock-in of TAD1 into phage SBSphiJ, which does not naturally encode this gene, rendered the phage fully resistant to Thoeris, showing that TAD1 alone confers the anti-Thoeris phenotype (FIG. 2B).

The identified TAD1 is a small protein (142 aa) of unknown function, with no recognizable protein domain. A search based on sequence homology identified over 750 TAD1 homologs in the integrated microbial genomes (IMG)³⁸ and the metagenomic gut virus (MGV)³⁹ databases (Table 6-7 hereinabove). Strikingly, all homologs of TAD1 resided either in phage genomes or in genomes of prophages integrated within bacterial genomes, indicating that this protein primarily executes a phage function. The discovered TAD1-encoding phages belong to multiple phage families, including Myoviridae, Podoviridae and Siphoviridae, and infect at least 60 species of bacteria from a diverse set of taxonomic phyla including Proteobacteria, Firmicutes and Cyanobacteria (FIG. 2D).

A phylogenetic analysis based on 256 unique TAD1 sequences showed that TAD1 proteins can be divided into four major clades (FIG. 2D). In some cases, phages encoding similar TAD1 proteins that were placed on the same phylogenetic clade belonged to different phage families, suggesting that TAD1 genes can be horizontally transferred between phages (FIG. 2D).

In the next step, 10 TAD1 homologs that span the phylogenetic diversity of the TAD1 family were selected, and each of them was cloned into Bacillus subtilis cells that express the Thoeris system from Bacillus cereus MSX-D12 (FIG. 2D). All 10 TAD1 family proteins were able to inhibit Thoeris, including homologs derived from phages that infect distant organisms such as Leptolyngbya sp., Opitutaceae sp. and Acinetobacter baumannii (FIGS. 2D-E and 11A-B). Combined together, these results reveal a large family of proteins utilized by phages to inhibit the activity of the Thoeris bacterial defense system.

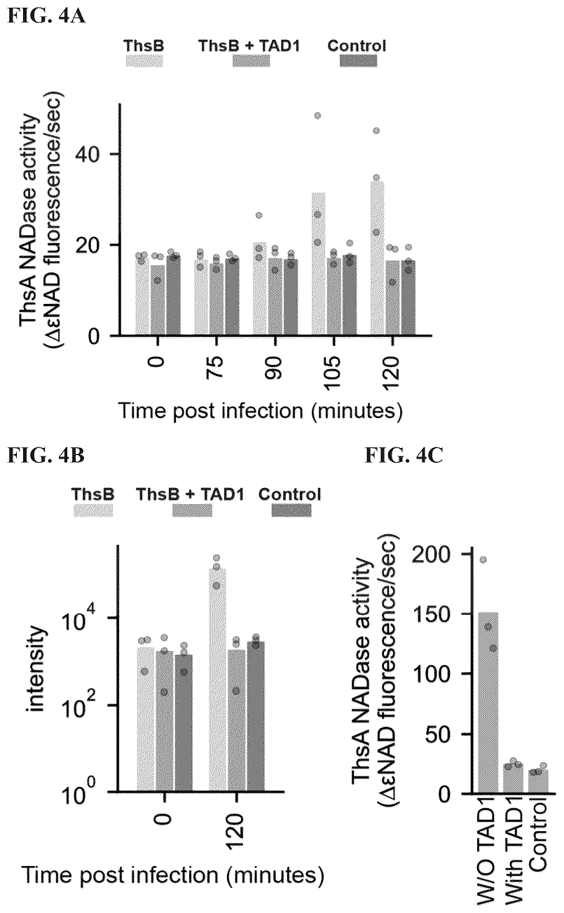

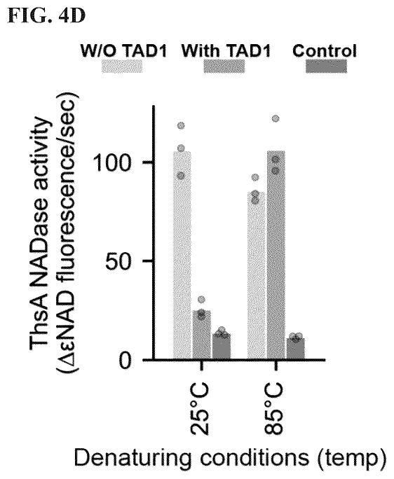

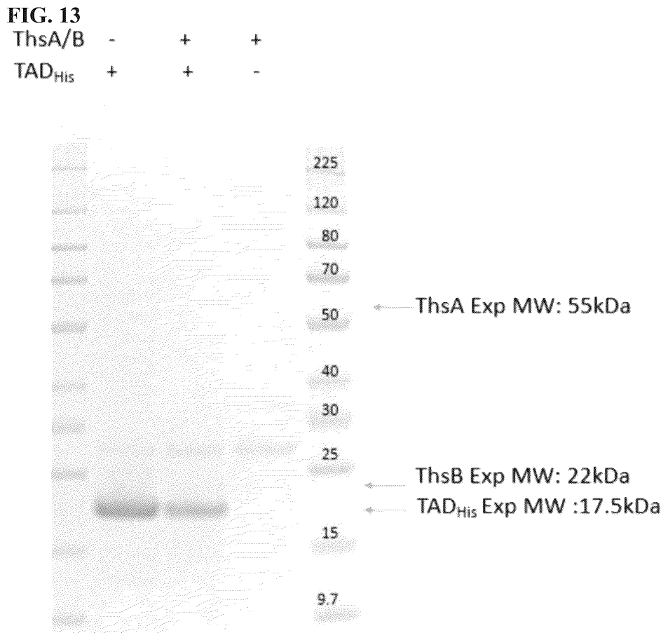

The mechanism of action of the Thoeris defense system was recently described in detail²⁸, opening a path to examine how TAD1 inhibits Thoeris. As most of the known anti-CRISPR proteins function by directly binding and inhibiting key proteins on the CRISPR-Cas complex, it was initially anticipated that TAD1 will bind one of the two Thoeris proteins, ThsA or ThsB. However, TAD1 did not co-immunoprecipitate with either ThsA or ThsB, implying that TAD1 inhibits Thoeris via a mechanism that does not require physical interaction with Thoeris proteins (FIG. 13). The hallmark of Thoeris defense system are TIR-domain proteins which produce the cADPR isomer signaling molecule that triggers the NADase activity of the Thoeris ThsA protein once they sense the infection²⁸. To test if this signaling molecule is produced in the presence of TAD1, cells expressing a Thoeris system from Bacillus cereus MSX-D12 in which ThsA was mutated in its NADase active site were used. These cells were infected by phage SBSphiJ, followed by lysis of the cells and filtration of the lysates to include only molecules smaller than 3 kDa. As expected, purified ThsA protein incubated with these lysates in vitro became a strong NADase, indicating that the TIR-domain ThsB protein produced the signaling molecule within the cell in response to SBSphiJ infection (FIG. 4A). However, lysates derived from cells in which TAD1 was co-expressed with the Thoeris system failed to activate ThsA in vitro, suggesting that the signaling molecule is not produced in Thoeris-infected cells that express TAD1 (FIG. 4A). Liquid chromatography followed by tandem mass spectrometry (LC-MSMS) confirmed that the signaling molecule was present in lysates of infected cells expressing ThsB, but absent in lysates derived from infected cells where ThsB was co-expressed with TAD1 (FIG. 4B). These results indicate that TAD1 works upstream of ThsA.

Next, the inventors experimented with a purified TAD1 homolog from Clostridioides mangenotii (SEQ ID NO: 269 (NA)/337 (AA)). To test if TAD1 can eliminate the signaling molecule from the lysate, 3 kDa filtered lysates were collected from cells expressing ThsB that were infected by phage SBSphiJ, and the filtrates were incubated with TAD1 for 10 minutes. Lysates incubated with TAD1 completely lost their ability to induce ThsA, demonstrating that TAD1 rapidly eliminates the signaling molecule from the lysate rather than inhibiting its production (FIG. 4C).

To examine if TAD1 is an enzyme or a chelator (a “sponge”) that strongly binds and sequesters the signaling v-cADPR molecule, the inventors set out to test whether, following the chelation, TAD1 denaturation could release the bound signaling molecule. To this end, TAD1 was first incubated with the signal-containing lysate for 10 minutes, and then denatured by exposure to 85° C. for 5 minutes. Following re-filtering of the medium it was found that this medium robustly activated ThsA in vitro (FIG. 4D). These results demonstrate that TAD1 binds and sequesters, but does not degrade, the Thoeris signaling molecule. Without being bound by theory, these results point to a mechanistic model in which TAD1 inhibits Thoeris defense by physically binding and sequestering the TIR-derived signaling molecule (cADPR isomer), thus preventing the activation of the Thoeris immune effector and mitigating Thoeris-mediated premature cell death.

### Example 3

### Had1 as an Anti-Hachiman Gene

The Hachiman defense system is widely distributed in genomes of bacterial and archaeal species, and was shown to protect against a broad range of phages¹⁹. The system encodes a protein with a predicted helicase domain, as well as an additional protein of unknown function.

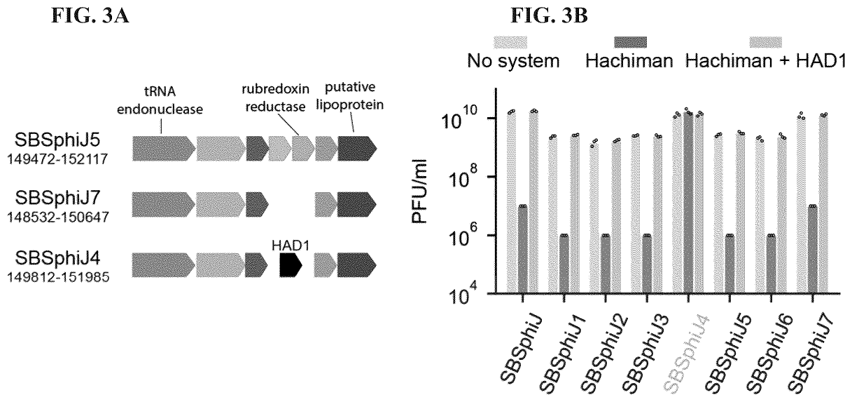

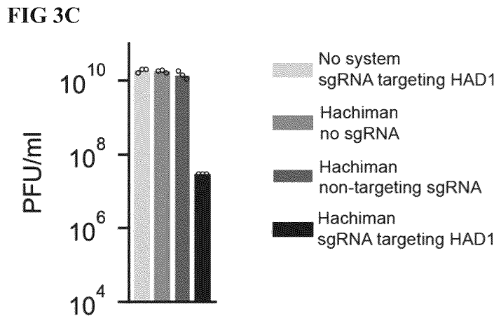

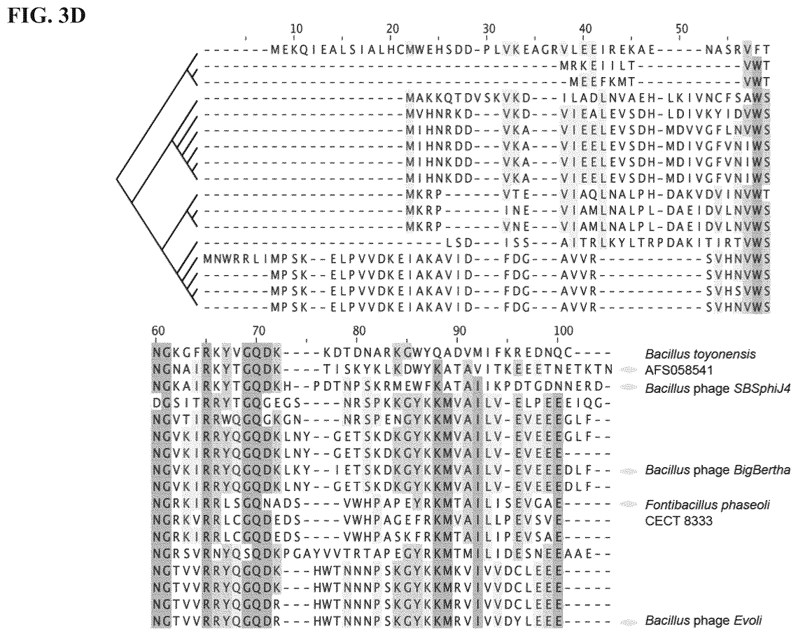

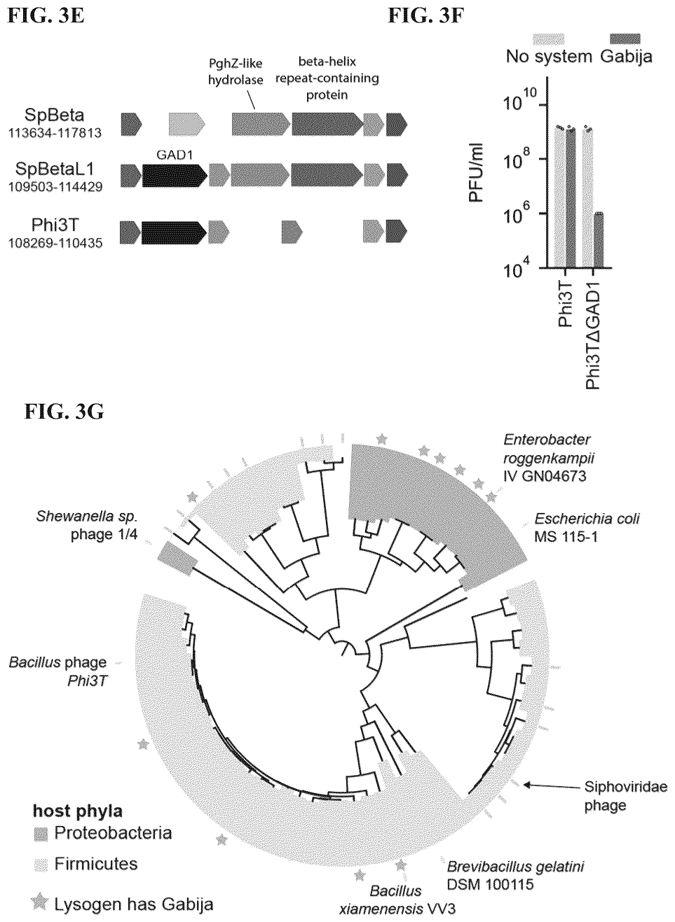

Three short genes of unknown function that were unique to phage SBSphiJ4, a phage that overcame Hachiman-mediated defense (FIG. 1C, Table 5 hereinbelow), were cloned into a B. subtilis strain that expresses the Hachiman system from B. cereus B4087¹⁹. One of these genes, referred to herein as “HAD1 (Hachiman Anti Defense 1)”, completely abolished Hachiman-mediated defense (FIGS. 3A-B). Further, silencing of HAD1 expression in SBSphiJ4 resulted in a phage that could infect control strains, but was blocked by Hachiman defense, supporting that HAD1 is responsible for the anti-Hachiman phenotype (FIG. 3C).

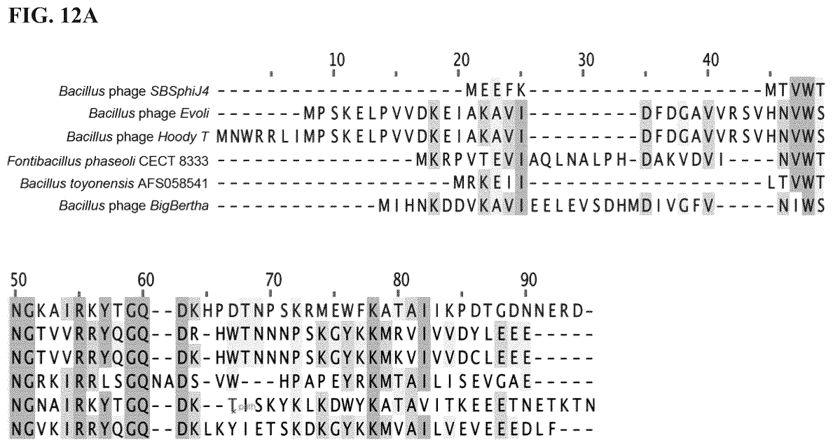

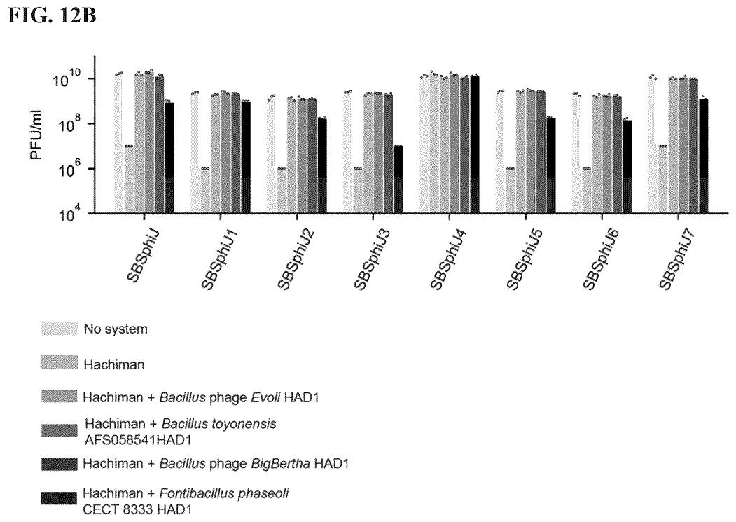

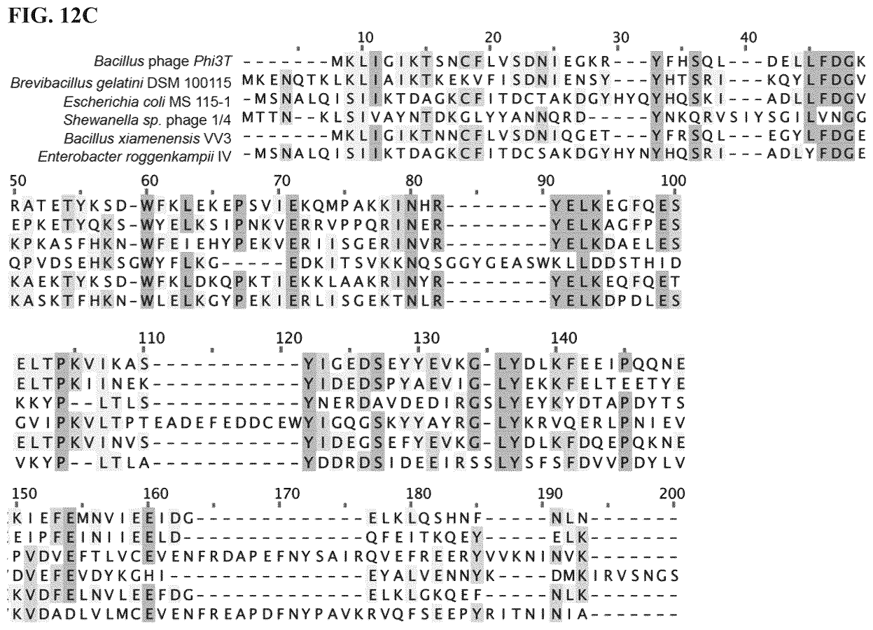

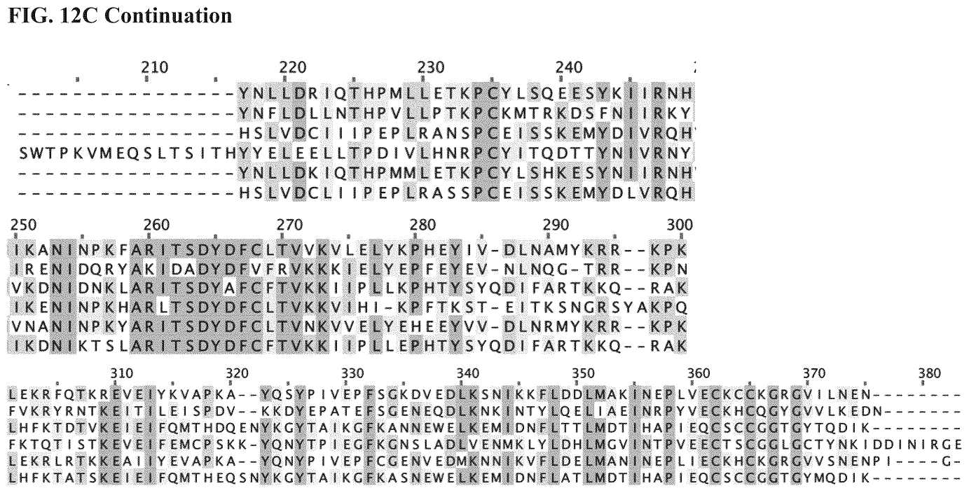

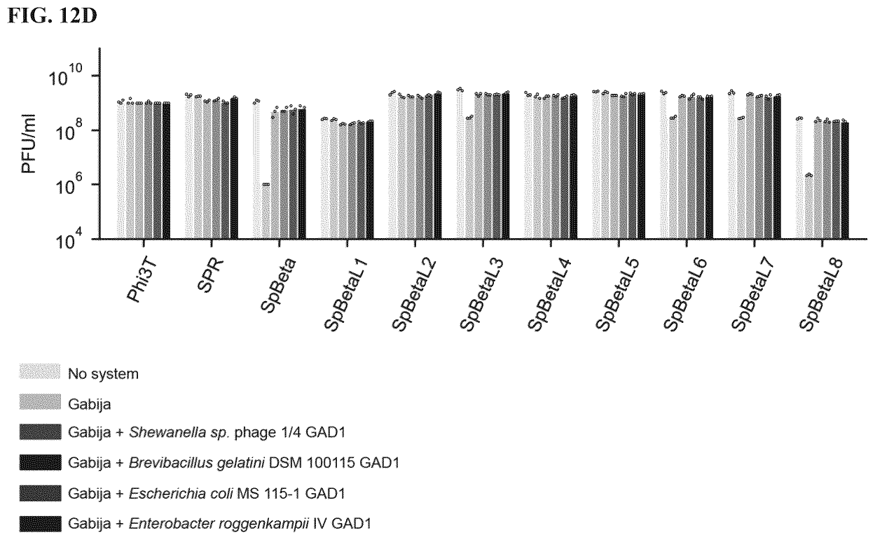

HAD1 is a short protein of 53aa, which does not show sequence homology to any protein of known function. 23 homologs of HAD1 were found in Bacillus phages as well as prophages integrated within Bacillus and Paenibacillus genomes (Table 8 hereinabove). Five homologs that span the protein sequence diversity of HAD1 were selected for further experimental examination (FIG. 12A). Four of these proteins efficiently inhibited the activity of Hachiman, and the fifth could not be cloned into Hachiman-expressing cells (FIGS. 3D and 12B). These results confirm that HAD1 is a Hachiman-inhibiting family of phage proteins.

### Example 4

### Gad1 as an Anti-Gabija Gene

The Gabija defense system is widely distributed in genomes of bacterial and archaeal species, and was shown to protect against a broad range of phages¹⁹. The system encodes a protein with a predicted helicase domain, as well as an additional protein of unknown function.

The Gabija defense system from B. cereus VD045 cloned within B. subtilis provided strong protection against some phages of the SpBeta group including SpBeta and SpBetaL8, and intermediate, weaker defense against phages SpBetaL6 and SpBetaL7 (FIG. 1C). The remaining seven phages of the SpBeta group were able to overcome Gabija-mediated defense. Two genes that were present in the seven Gabija-overcoming phages were missing from phages that could not overcome Gabija defense (FIGS. 3E and 9; and Table 5 hereinbelow). One of these genes, referred to herein as “GAD1 (Gabija Anti Defense 1)” could not be cloned into Gabija-expressing cells, and the second gene did not show a Gabija-inhibiting phenotype when co-expressed with Gabija. To examine the possible function of GAD1, the inventors took advantage of the fact that SpBeta-like phages are temperate phages that can be genetically manipulated when integrated into the bacterial genome, and knocked out the GAD1 gene from the genome of phage phi3T. The results show that phi3T knocked out for GAD1 was no longer able to overcome Gabija defense, suggesting that this gene inhibits Gabija (FIG. 3F).

GAD1 is a 295aa-long protein that does not show direct sequence similarity to proteins of known function. 94 homologs of GAD1, which were distributed in genomes of various phages and prophages infecting host bacteria from the phyla Proteobacteria and Firmicutes were found (FIG. 3G, Tables 9-10 hereinabove). Interestingly, many of the GAD1-contaning prophages were integrated in bacterial genomes that also encoded the Gabija system, suggesting that GAD1 enabled these phages to overcome Gabija-mediated defense of their hosts (FIG. 3G). Five GAD1 homologs from phages infecting Shewanella sp., Enterobacter roggenkampii, Escherichia coli, Brevibacillus gelatini and Bacillus xiamenensis were selected for further experimental examination (FIGS. 3G and 12C).

These selected GAD1 homologs were successfully cloned to Gabija-containing cells, and all five genes efficiently inhibited the Gabija defense system (FIG. 12D).

### Example 5

### Dsad1 as an Anti-Dsr2 Gene

SIR2-domain proteins, or sirtiuins, are found in organisms ranging from bacteria to humans. These proteins have been widely studied in yeast and mammals, where they were shown to regulate transcription repression, recornbination, DNA repair and cell cycle processes¹, In eukaryotes, SIR2 domains were shown to possess enzymatic activities, and function either as protein deacetylases or ADP ribosyltransferases (Yuan, H. and Marmorstein, R. (2012) J. Biol. Chem., 287, 42428-42435). In bacteria, SIR2 domains were described as commonly taking part in defense systems that protect against phages (Gao, L., et al. (2020) Science, 369, 1077-1084). These domains are associated with multiple different defense systems, including prokaryotic argonautes (pAgo) (Swarts, D. C., et al. (2014) Nat. Struct. Mol. Biol., 21, 743-753), Thoeris (Doron, S., et al. (2018) Science (80-.), 359, eaar4120; Ofir, G., et al. (2021) bioRxiv, 10.1101/2021.01.06.425286), AVAST (Gao, L., et al. (2020) Science, 369, 1077-1084), DSR (Gao, L., et al. (2020) Science, 369, 1077-1084) and additional systems. The SIR2 domain in the Thoeris defense system was shown to be an NADase responsible for depleting NAD⁺ from the cell once phage infection has been sensed (Ofir, G., et al. (2021) bioRxiv, 10.1101/2021.01.06.425286). However, it is currently unknown whether SIR2 domains in other defense systems perform a similar function, or whether they have other roles in phage defense.

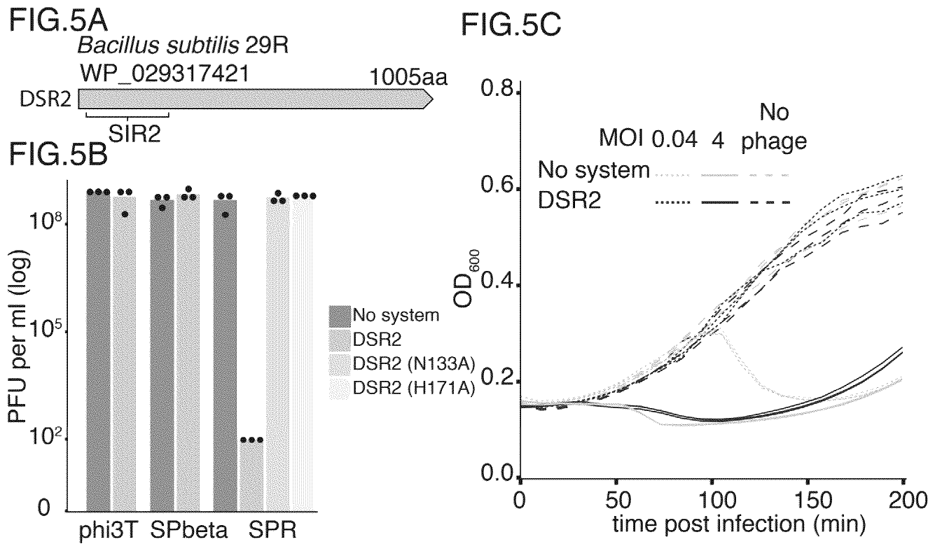

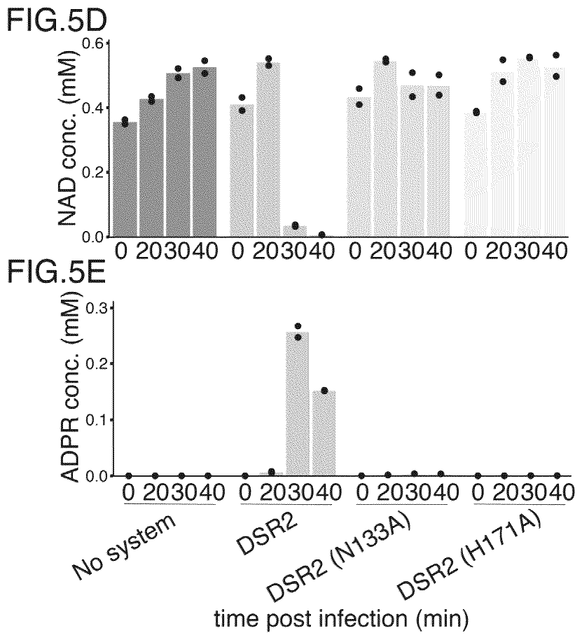

To study the role of SIR2 domains in bacterial defense, the inventors focused on DSR2, a minimal defense system that includes a single protein with an N-terminal SIR2 domain and no additional identifiable domains (FIG. 5A). To this end, the DSR2 gene from Bacillus subtilis 29R (SEQ ID NO: 746) together with its native promoter was cloned into the genome of B. subtilis BEST7003 which naturally lacks this gene, and found that DSR2 protected B. subtilis against SPR (FIG. 5B). Point mutations predicted to inactivate the active site of the SIR2 domain (N133A and H171A) abolished defense, suggesting that the enzymatic activity of SIR2 is essential for DSR2 defense (FIG. 5B). Following, the inventors tested whether DSR2 defends via abortive infection, a process that involves premature death or growth arrest of the infected cell, preventing phage replication and spread to nearby cells (Lopatina, A., Tal, N. and Sorek, R. (2020) Annu. Rev. Virol., 7(1):371-384). When infected in liquid media, DSR2-containing bacterial cultures demised if the culture was infected by phage in high multiplicity of infection (MOI), similar to DSR2-lacking cells (FIG. 5C). In low MOI infection, DSR2-lacking control cultures collapsed but DSR-containing bacteria survived (FIG. 5C). This phenotype is a hallmark of abortive infection, in which infected bacteria do not survive but also do not produce phage progeny. To test whether DSR2 manipulates the NAD⁺ content in the cell during phage infection, mass spectrometry was used to monitor NAD⁺ levels in infected cells at various time points following initial infection. When DSR2-containing cells were infected by phage, cellular NAD⁺ sharply decreased between 20 and 30 minutes from the onset of infection (FIG. 5D). NAD⁺ levels were not changed in cells in which the SIR2 active site was mutated, or in DSR2-lacking control cells, suggesting that the SIR2 domain is responsible to the observed NAD⁺ depletion (FIG. 5D). In parallel with NAD⁺ depletion, accumulation of ADP-ribose (ADPR), the product of NAD⁺ cleavage, was observed (FIG. 5E). These results demonstrate that DSR2 is an abortive infection protein that causes NAD⁺ depletion in infected cells.

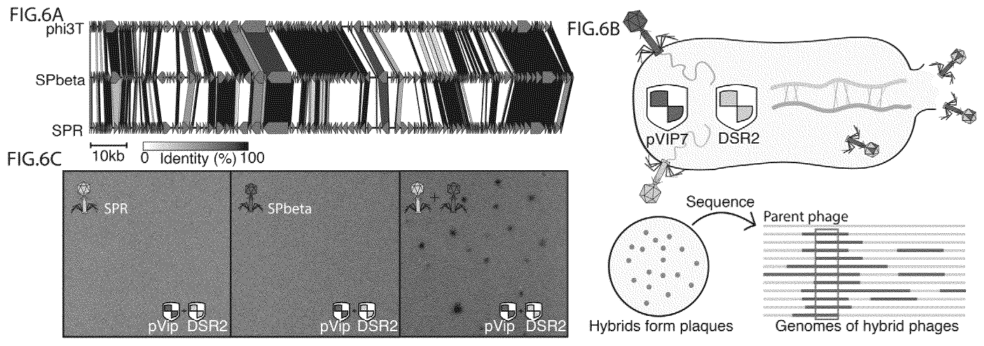

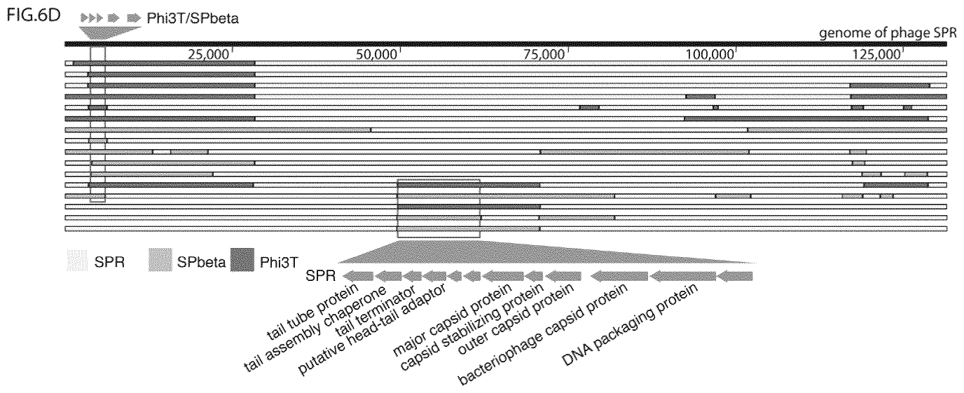

The DSR2 defense system strongly protected B. subtilis cells against SPR, a phage belonging the SPBeta group of phages (FIG. 5B). However, the defense gene failed to protect against phages phi3T and SPBeta, although both these phages also belong to the SPBeta group (FIG. 5B). This observation suggests that phages phi3T and SpBeta either encode genes that inhibit DSR2, or lack genes that are recognized by DSR2 and trigger its NADase activity. SPR, phi3T and SPbeta all share substantial genomic regions with high sequence homology (FIG. 6A). It was therefore reasoned that co-infecting cells with SPR and phi3T may result in recombination-mediated genetic exchange between the phages, which would enable pinpointing genes that allow escape from DSR2 defense when acquired by SPR (FIG. 6B).

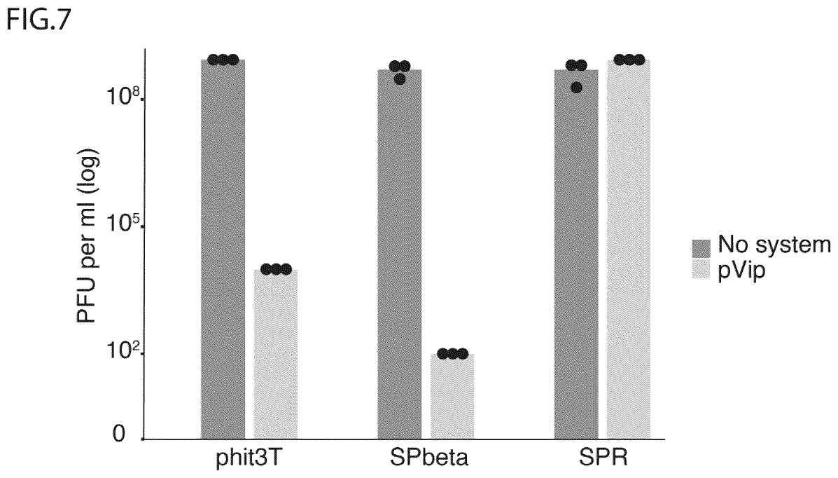

To generate a bacterial host that would select for such genetic exchange events, a prokaryotic viperin homolog (pVip) from Fibrobacter sp. UWT3 (Bernheim, A., et al. (2021) Nature, 589(7840):120-124) was also cloned into DSR2-containing cells. This pVip protein, when expressed alone in B. subtilis, protected it from phi3T and SpBeta but not from SPR, a defense profile which is opposite to the DSR2 profile in terms of the affected phages (FIG. 7). Accordingly, none of the three SPBeta-group phages could form plaques on the strain that expressed both defensive genes. However, when simultaneously infecting the double defense strain with SPR and phi3T or SpBeta, plaque forming hybrids were readily obtained, indicating that these hybrid phages recombined and have acquired a combination of genes enabling them to escape both DSR2 and pVip (FIG. 6C).

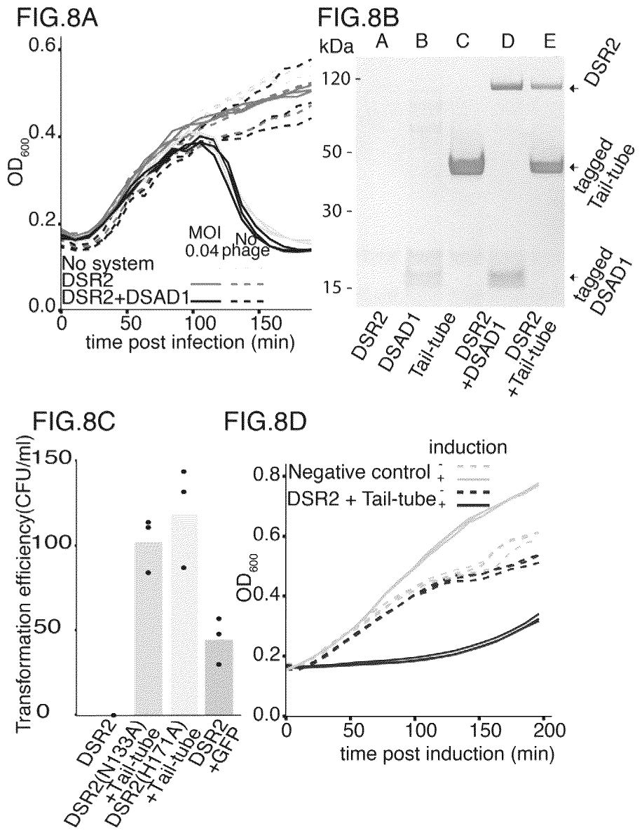

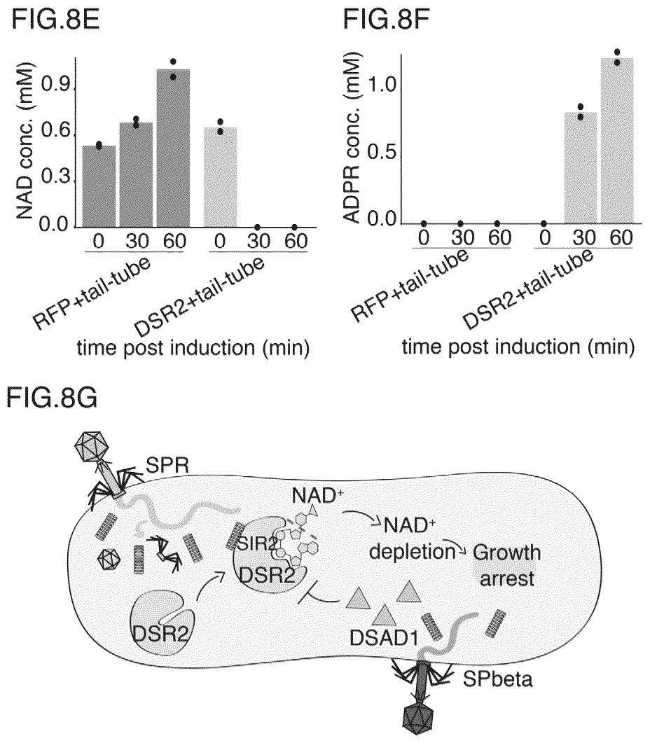

Following, 16 such hybrid phages were isolated and sequenced, their genomes assembled, and these genomes were compared to the genome of the parent SPR phage (FIG. 6D). This led to the identification of two genomic segments that were repeatedly acquired by SPR phages. Acquisition of either of these segments from a genome of a co-infecting phage rendered SPR resistant to DSR2 (FIG. 6D). The first segment included several small genes of unknown function that are present in phages phi3T and SPBeta but not in the wild type SPR phage. Hence, these genes ability to inhibit DSR2 was tested. One of the genes (SEQ ID NO: 738), referred to herein as “DSAD1 (DSR Anti Defense 1)”, when co-expressed with DSR2 in the same cell, rendered DSR2 inactive, suggesting that this phage gene encodes an anti-DSR2 protein (FIG. 8A). The anti-DSR2 gene encodes a 120aa protein (SEQ ID NO: 739), with no identifiable homology to proteins of known function. Co-expression of DSR2 and tagged DSAD1, followed by pulldowns, showed direct interaction between the two proteins (FIG. 8B). These results indicate DSAD1 is a phage protein that binds and inhibits DSR2.

Following, the second genomic segment that, when acquired, allowed phage hybrids to escape DSR2 was examined. In the parent SPR phage, this region spans a set of genes encoding phage structural proteins. Hybrid phages in which the original genes were replaced by their homologs from SpBeta or phi3T become resistant to DSR2 (FIG. 6D). It was hypothesized that one of the structural proteins in phage SPR is recognized by DSR2 to activate its defense, and when this protein is replaced by its homolog from another phage, recognition no longer occurs. To test this hypothesis, each of the structural genes found in the original DNA segment in SPR was cloned into B. subtilis cells that also express DSR2. One of these genes, encoding a tail-tube protein, failed to clone into DSR2-expressing cells but was readily cloned into cells in which the DSR2 active site was mutated (FIG. 8C). To further test if co-expression of DSR2 and the SPR tail tube protein is toxic to bacteria, each of these genes was cloned under an inducible promoter within E. coli cells. Indeed, growth was rapidly arrested in cells in which the expression of both genes was induced (FIG. 8D), and these cells became depleted of NAD+(FIG. 8E-F). Furthermore, pulldown assays showed that DSR2 directly binds the tail tube protein of phage SPR (FIG. 8B). Without being bound by theory, these results demonstrate that the tail tube protein of phage SPR is directly recognized by the defense protein DSR2, and that this recognition triggers the NADase activity of DSR2 and results in growth arrest (FIG. 8G).

### Example 6

### Tad2 as an Anti-Thoeris Gene

The Thoeris anti-phage system provides strong defense against phages SPO1L1, SPO1L2, SPO1L4 and SPO1L5, while the genomically-similar phages SPO1 and SPO1L3 partially escape Thoeris mediated defense (FIG. 14A). One gene was identified as unique to phages SPO1 and SPO1L3 [SEQ ID NO: 757 (NA)/1046 (AA)]. To test if this gene is a novel Thoeris inhibitor, it was cloned under the control of an inducible promoter into B. subtilis cells that also express the Thoeris system from Bacillus cereus MSX-D12 [SEQ ID NO: 741 (NA)/1296-1297 (AA)]. Indeed, this gene, referred to herein as “TAD2 (Thoeris Anti Defense 2)”, robustly inhibited the activity of Thoeris, as phages that were normally blocked by Thoeris were able to infect Thoeris-expressing cells if these cells also expressed TAD2 (FIG. 14A). Further, silencing the expression of TAD2 in SPO1 using dCas9³⁷ caused SPO1 to be blocked by Thoeris, verifying that TAD2 is the gene responsible for the Thoeris-inhibiting phenotype of SPO1 (FIG. 14B).

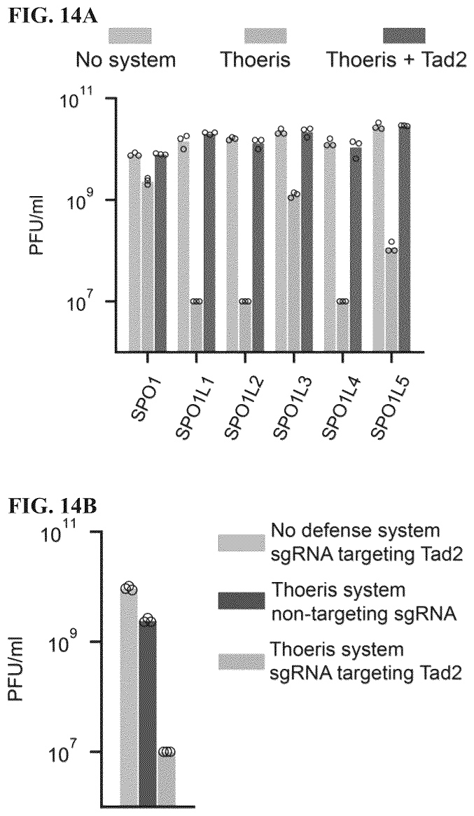

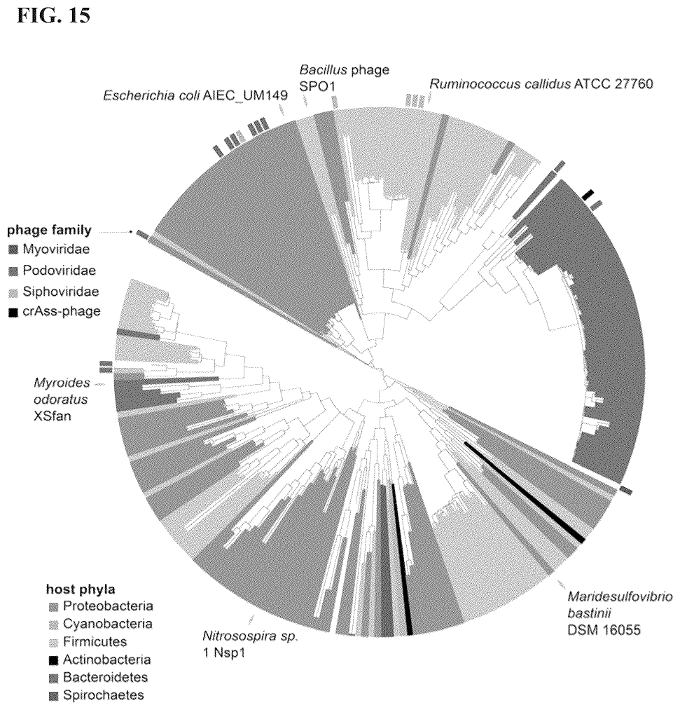

Following, 250 unique homologs of TAD2, which were distributed in genomes of various phages and prophages infecting host bacteria from 6 different phyla were found (FIG. 15, Tables 11-12 hereinabove).

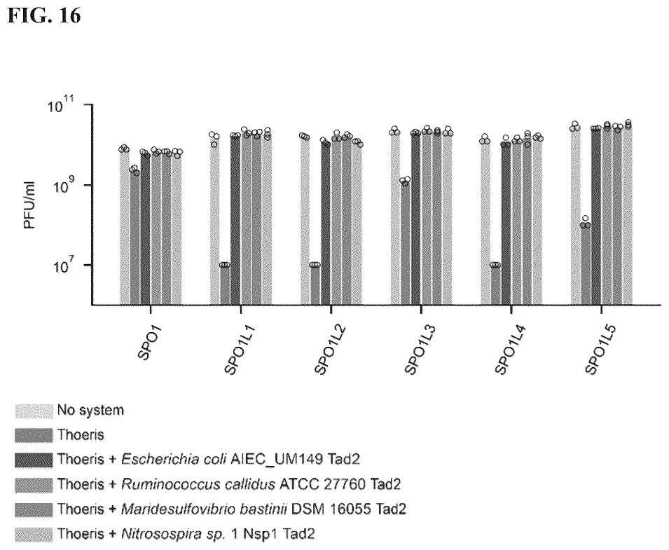

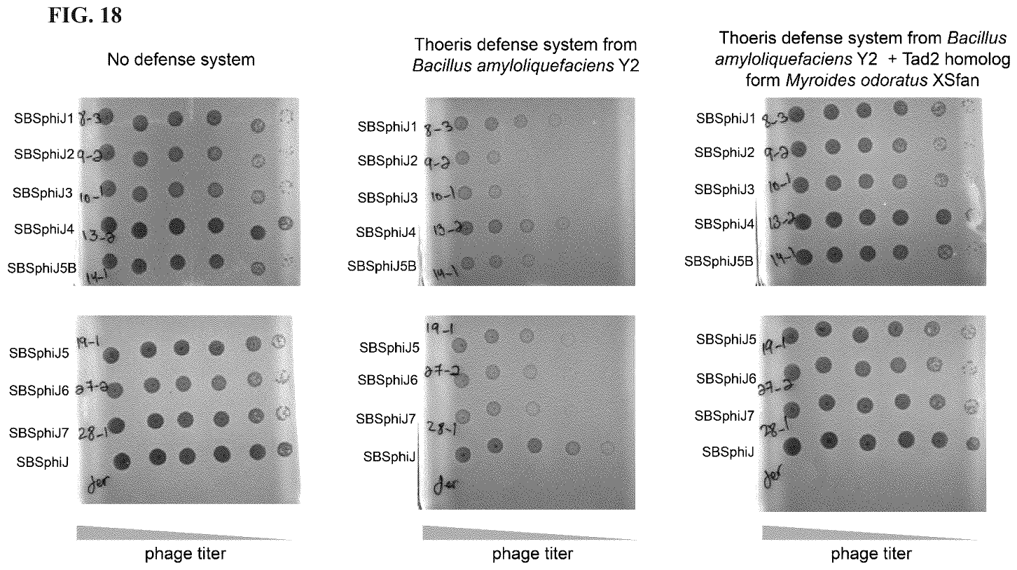

In the next step, four TAD2 homologs from phages infecting Escherichia coli, Ruminococcus callidus, Maridesulfovibrio bastinii and Nitrosospira sp. cloned under an inducible promoter into cells expressing the Thoeris defense system from Bacillus cereus MSX-D12 [SEQ ID NO: 741 (NA)/1296-1297 (AA)] efficiently inhibited the Thoeris defense system (FIG. 16). Other TAD2 homologs efficiently inhibited the Thoeris defense systems from Bacillus amyloliquefaciens Y2 [SEQ ID NO: 1300 (NA)/1301-1302 (AA)] (FIG. 18).

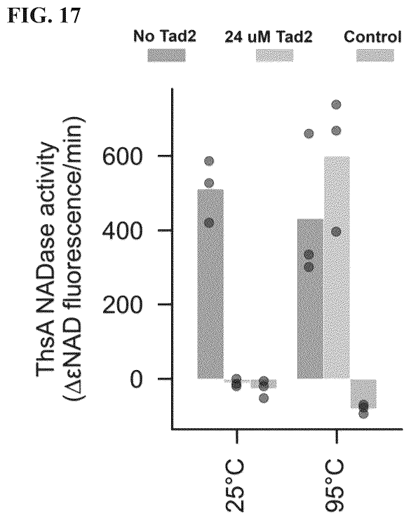

To examine if TAD2 is also a chelator (a “sponge”) that strongly binds and sequesters the TIR-derived molecule, the present inventors tested whether, following the chelation, TAD2 denaturation could release the bound signaling molecule. To this end, TAD2 was first incubated with the TIR-derived signal (1″-3′gcADPR) for 10 minutes, and then denatured by exposure to 95° C. for 5 minutes. Following re-filtering of the medium it was found that this medium robustly activated ThsA in vitro (FIG. 17). These results demonstrate that similarly to TAD1, TAD2 binds and sequesters the Thoeris signaling molecule, thus decoupling phage sensing and immune effector activation and rendering Thoeris inactive.

TABLE 1
SBSphiJ % genome overlap

| | Query | | | | | | | |
|---|---|---|---|---|---|---|---|---|
| Target | SBSphiJ3 | SBSphiJ6 | SBSphiJ2 | SBSphiJ | SBSphiJ4 | SBSphiJ1 | SBSphiJ5 | SBSphiJ7 |
| SBSphiJ3 | 100 | 94 | 97 | 98 | 91 | 94 | 91 | 92 |
| SBSphiJ6 | 95 | 100 | 95 | 95 | 94 | 96 | 91 | 92 |
| SBSphiJ2 | 96 | 92 | 100 | 97 | 90 | 93 | 90 | 90 |
| SBSphiJ | 96 | 93 | 97 | 100 | 92 | 93 | 91 | 92 |
| SBSphiJ4 | 92 | 94 | 92 | 93 | 100 | 93 | 89 | 92 |
| SBSphiJ1 | 95 | 96 | 95 | 94 | 92 | 100 | 92 | 93 |
| SBSphiJ5 | 92 | 91 | 92 | 92 | 89 | 92 | 100 | 92 |
| SBSphiJ7 | 93 | 92 | 93 | 94 | 92 | 94 | 93 | 100 |

TABLE 2
SBSphiJ sequence identity

| | SBSphiJ3 | SBSphiJ6 | SBSphiJ2 | SBSphiJ | SBSphiJ4 | SBSphiJ1 | SBSphiJ5 | SBSphiJ7 |
|---|---|---|---|---|---|---|---|---|
| SBSphiJ3 | 100 | 99.3 | 98.9 | 98.1 | 97.6 | 98.7 | 97.1 | 97.9 |
| SBSphiJ6 | | 100 | 99.5 | 98.3 | 96.5 | 98.8 | 97.3 | 95.9 |
| SBSphi.J2 | | | 100 | 99.4 | 98.1 | 98.9 | 96.7 | 98 |
| SBSphiJ | | | | 100 | 97.9 | 98.2 | 96.7 | 94.2 |
| SBSphiJ4 | | | | | 100 | 96.7 | 96.7 | 97.2 |
| SBSphiJ1 | | | | | | 100 | 96.7 | 96.2 |
| SBSphiJ5 | | | | | | | 100 | 95.3 |
| SBSphiJ7 | | | | | | | | 100 |

TABLE 3
SpBeta % genome overlap

| | Query | | | | | | | | | | |
|---|---|---|---|---|---|---|---|---|---|---|---|
| Target | SpBetaL2 | SPR | SpBetaL3 | SpBetaL8 | SpBetaL7 | SpBetaL6 | SpBeta | phi3T | SpBetaL1 | SpBetaL4 | SpBetaL5 |
| SpBetaL2 | 100 | 81 | 66 | 57 | 52 | 53 | 55 | 46 | 55 | 54 | 45 |
| SPR | 83 | 100 | 64 | 57 | 52 | 51 | 54 | 45 | 54 | 52 | 48 |
| SpBetaL3 | 67 | 63 | 100 | 50 | 45 | 46 | 44 | 62 | 49 | 45 | 60 |
| SpBetaL8 | 60 | 58 | 52 | 100 | 70 | 69 | 65 | 61 | 68 | 69 | 60 |
| SpBetaL7 | 53 | 52 | 46 | 68 | 100 | 96 | 66 | 53 | 66 | 77 | 57 |
| SpBetaL6 | 54 | 51 | 46 | 67 | 96 | 100 | 66 | 53 | 69 | 82 | 54 |
| SpBeta | 55 | 53 | 43 | 62 | 64 | 65 | 100 | 58 | 60 | 67 | 49 |
| phi3T | 48 | 46 | 64 | 60 | 54 | 55 | 61 | 100 | 56 | 55 | 69 |
| SpBetaL1 | 55 | 54 | 49 | 65 | 64 | 67 | 61 | 53 | 100 | 67 | 55 |
| SpBetaL4 | 56 | 53 | 46 | 68 | 79 | 83 | 70 | 54 | 69 | 100 | 52 |
| SpBetaL5 | 47 | 49 | 62 | 60 | 59 | 56 | 52 | 70 | 58 | 53 | 100 |

TABLE 4
SpBeta sequence identity

| | SpBetaL2 | SPR | SpBetaL3 | SpBetaL8 | SpBetaL7 | SpBetaL6 | SpBeta | phi3T | SpBetaL1 | SpBetaL4 | SpBetaL5 |
|---|---|---|---|---|---|---|---|---|---|---|---|
| SpBetaL2 | 100 | 99.7 | 98.1 | 97.7 | 97.4 | 97.4 | 96.9 | 96.9 | 96.5 | 91.7 | 97.9 |
| SPR | | 100 | 98.6 | 97.7 | 97.1 | 97.1 | 97 | 97.1 | 96.4 | 91.5 | 97.7 |
| SpBetaL3 | | | 100 | 97.2 | 96.9 | 96.9 | 97.6 | 99.3 | 97.2 | 90 | 98.1 |
| SpBetaL8 | | | | 100 | 97.2 | 97.2 | 97.5 | 97.5 | 96.6 | 96.6 | 96.3 |
| SpBetaL7 | | | | | 100 | 100 | 96.9 | 96.5 | 96.3 | 93.9 | 97 |
| SpBetaL6 | | | | | | 100 | 96.9 | 96.7 | 97.5 | 99.7 | 98.1 |
| SpBeta | | | | | | | 100 | 99.7 | 96.2 | 96.6 | 97.3 |
| phi3T | | | | | | | | 100 | 96.2 | 96.6 | 98.7 |
| SpBetaL1 | | | | | | | | | 100 | 97.3 | 96 |
| SpBetaL4 | | | | | | | | | | 100 | 96.9 |
| SpBetaL5 | | | | | | | | | | | 100 |

TABLE 5
Candidate anti-defense genes

| | ORF | additional | candidate | verified as | AA | psi-blast functional | DNA sequence | AA sequence |
|---|---|---|---|---|---|---|---|---|
| phage | # | name | for system | anti-defense | length | annotation | SEQ ID NO: | SEQ ID NO: |
| SBSphiJ7 | 74 | | Thoeris | no | 216 | unknown | 1 | 18 |
| SBSphiJ7 | 142 | | Thoeris | no | 65 | unknown | 2 | 19 |
| SBSphiJ7 | 146 | | Thoeris | no | 67 | unknown | 3 | 20 |
| SBSphiJ7 | 155 | | Thoeris | no | 708 | DNA polymerase | 4 | 21 |
| SBSphiJ7 | 158 | TAD1 | Thoeris | yes | 142 | unknown | 5 | 22 |
| SBSphiJ7 | 204 | | Thoeris | no | 237 | HNH endonuclease | 6 | 23 |
| SBSphiJ4 | 27 | | Hachiman | no | 79 | Minor tail protein | 7 | 24 |
| SBSphiJ4 | 28 | | Hachiman | no | 54 | unknown | 8 | 25 |
| SBSphiJ4 | 156 | | Hachiman | no | 100 | DNA polymerase | 9 | 26 |
| SBSphiJ4 | 157 | | Hachiman | no | 230 | HNH endonuclease | 10 | 27 |
| SBSphiJ4 | 158 | | Hachiman | no | 219 | DNA polymerase | 11 | 28 |
| SBSphiJ4 | 181 | | Hachiman | no | 44 | RNA polymerase | 12 | 29 |
| SBSphiJ4 | 194 | | Hachiman | no | 37 | PKHD-type hydroxylase | 13 | 30 |
| SBSphiJ4 | 232 | | Hachiman | no | 146 | unknown | 14 | 31 |
| SBSphiJ4 | 243 | HAD1 | Hachiman | yes | 53 | unknown | 15 | 32 |
| Phi3T | 158 | GAD1 | Gabija | yes | 295 | unknown | 16 | 33 |
| Phi3T | 159 | | Gabija | no | 90 | unknown | 17 | 34 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited Throughout the Application)

1. Labrie, S. J., Samson, J. E. & Moineau, S. Bacteriophage resistance mechanisms. Nat. Rev. Microbiol. 8, 317-327 (2010).
2. Stem, A. & Sorek, R. The phage-host arms race: Shaping the evolution of microbes. BioEssays 33, 43-51 (2011).
3. Dy, R. L., Richter, C., Salmond, G. P. C. & Fineran, P. C. Remarkable Mechanisms in Microbes to Resist Phage Infections. Annu. Rev. Virol. 1, 307-331 (2014).
4. Samson, J. E., Magadin, A. H., Sabri, M. & Moineau, S. Revenge of the phages: defeating bacterial defences. Nat. Rev. Microbiol. 11, 675-687 (2013).
5. Ofir, G. & Sorek, R. Contemporary Phage Biology: From Classic Models to New Insights. Cell 172, 1260-1270 (2018).
6. Atanasiu, C. Interaction of the ocr gene 0.3 protein of bacteriophage T7 with EcoKI restriction/modification enzyme. Nucleic Acids Res. 30, 3936-3944 (2002).
7. Walkinshaw, M. et al. Structure of Ocr from Bacteriophage T7, a Protein that Mimics B-Form DNA. Mol. Cell 9, 187-194 (2002).
8. Iida, S., Streiff, M. B., Bickle, T. A. & Arber, W. Two DNA antirestriction systems of bacteriophage P1, darA, and darB: characterization of darA- phages. Virology 157, 156-166 (1987).
9. Drozdz, M., Piekarowicz, A., Bujnicki, J. M. & Radlinska, M. Novel non-specific DNA adenine methyltransferases. Nucleic Acids Res. 40, 2119-2130 (2012).
10. Studier, F. W. & Movva, N. R. SAMase gene of bacteriophage T3 is responsible for overcoming host restriction. J. Virol. 19, 136-145 (1976).
11. Thavalingam, A. et al. Inhibition of CRISPR-Cas9 ribonucleoprotein complex assembly by anti-CRISPR AcrIIC2. Nat. Commun. 10, 2806 (2019).
12. Lu, W.-T., Trost, C. N., Müller-Esparza, H., Randau, L. & Davidson, A. R. Anti-CRISPR AcrIF9 functions by inducing the CRISPR-Cas complex to bind DNA non-specifically. Nucleic Acids Res. 49, 3381-3393 (2021).
13. Bondy-Denomy, J. et al. Multiple mechanisms for CRISPR-Cas inhibition by anti-CRISPR proteins. Nature 526, 136-139 (2015).
14. Stanley, S. Y. & Maxwell, K. L. Phage-Encoded Anti-CRISPR Defenses. Annu. Rev. Genet. 52, 445-464 (2018).
15. Li, Y. & Bondy-Denomy, J. Anti-CRISPRs go viral: The infection biology of CRISPR-Cas inhibitors. Cell Host Microbe 29, 704-714 (2021).
16. Jia, N. & Patel, D. J. Structure-based functional mechanisms and biotechnology applications of anti-CRISPR proteins. Nat. Rev. Mol. Cell Biol. 22, 563-579 (2021).
17. Otsuka, Y. & Yonesaki, T. Dmd of bacteriophage T4 functions as an antitoxin against Escherichia coli LsoA and RnlA toxins. Mol. Microbiol. 83, 669-681 (2012).
18. Blower, T. R., Evans, T. J., Przybilski, R., Fineran, P. C. & Salmond, G. P. C. Viral Evasion of a Bacterial Suicide System by RNA-Based Molecular Mimicry Enables Infectious Altruism. PLoS Genet. 8, e1003023 (2012).
19. Doron, S. et al. Systematic discovery of antiphage defense systems in the microbial pangenome. Science (80-.). 359, eaar4120 (2018).
20. Gao, L. et al. Diverse enzymatic activities mediate antiviral immunity in prokaryotes. Science (80-.). 369, 1077-1084 (2020).
21. Cohen, D. et al. Cyclic GMP-AMP signalling protects bacteria against viral infection. Nature 574, 691-695 (2019).
22. Millman, A. et al. Bacterial Retrons Function In Anti-Phage Defense. Cell 183, 1551-1561.e12 (2020).
23. Bernheim, A. et al. Prokaryotic viperins produce diverse antiviral molecules. Nature 589, 120-124 (2021).
24. Bernheim, A. & Sorek, R. The pan-immune system of bacteria: antiviral defence as a community resource. Nat. Rev. Microbiol. 18, 113-119 (2020).
25. Whiteley, A. T. et al. Bacterial cGAS-like enzymes synthesize diverse nucleotide signals. Nature 567, 194-199 (2019).
26. Lau, R. K. et al. Structure and Mechanism of a Cyclic Trinucleotide-Activated Bacterial Endonuclease Mediating Bacteriophage Immunity. Mol. Cell 77, 723-733.e6 (2020).
27. Millman, A., Melamed, S., Amitai, G. & Sorek, R. Diversity and classification of cyclic-oligonucleotide-based anti-phage signalling systems. Nat. Microbiol. 5, 1608-1615 (2020).
28. Ofir, G. et al. Antiviral activity of bacterial TIR domains via signaling molecules that trigger cell death. bioRxiv 2021.01.06.425286 (2021) doi:10.1101/2021.01.06.425286.
29. Kronheim, S. et al. A chemical defence against phage infection. Nature 564, 283-286 (2018).
30. Bobonis, J. et al. Phage proteins block and trigger retron toxin/antitoxin systems. bioRxiv 2020.06.22.160242 (2020) doi:10.1101/2020.06.22.160242.
31. Bondy-Denomy, J., Pawluk, A., Maxwell, K. L. & Davidson, A. R. Bacteriophage genes that inactivate the CRISPR/Cas bacterial immune system. Nature 493, 429-432 (2013).
32. Pawluk, A., Bondy-Denomy, J., Cheung, V. H. W., Maxwell, K. L. & Davidson, A. R. A New Group of Phage Anti-CRISPR Genes Inhibits the Type I-E CRISPR-Cas System of Pseudomonas aeruginosa. MBio 5, (2014).
33. Rauch, B. J. et al. Inhibition of CRISPR-Cas9 with Bacteriophage Proteins. Cell 168, 150-158.e10 (2017).
34. Warner, F. D., Kitos, G. A., Romano, M. P. & Hemphill, H. E. Characterization of SPP: a temperate bacteriophage from Bacillus subtilis 168M. Can. J. Microbiol. 23, 45-51 (1977).
35. Tucker, R. G. Acquisition of Thymidylate Synthetase Activity by a Thymine-requiring Mutant of Bacillus subtilis following Infection by the Temperate Phage 3. J. Gen. Virol. 4, 489-504 (1969).
36. Noyer-Weidner, M., Jentsch, S., Pawlek, B., Günthert, U. & Trautner, T. A. Restriction and modification in Bacillus subtilis: DNA methylation potential of the related bacteriophages Z, SPR, SP beta, phi 3T, and rho 11. J. Virol. 46, 446-453 (1983).
37. Bikard, D. et al. Programmable repression and activation of bacterial gene expression using an engineered CRISPR-Cas system. Nucleic Acids Res. 41, 7429-7437 (2013).
38. Chen, I.-M. A. et al. IMG/M v.5.0: an integrated data management and comparative analysis system for microbial genomes and microbiomes. Nucleic Acids Res. 47, D666-D677 (2019).
39. Nayfach, S. et al. Metagenomic compendium of 189,680 DNA viruses from the human gut microbiome. Nat. Microbiol. 6, 960-970 (2021).
40. Bacteriophages. vol. 501 (Humana Press, 2009).
41. Mazzocco, A., Waddell, T. E., Lingohr, E. & Johnson, R. P. Enumeration of Bacteriophages Using the Small Drop Plaque Assay System. in 81-85 (2009). doi:10.1007/978-1-60327-164-6_9.
42. Baym, M. et al. Inexpensive Multiplexed Library Preparation for Megabase-Sized Genomes. PLoS One 10, e0128036 (2015).
43. Lee, T. S. et al. BglBrick vectors and datasheets: A synthetic biology platform for gene expression. J. Biol. Eng. 5, 12 (2011).
44. Martin, M. Cutadapt removes adapter sequences from high-throughput sequencing reads. EMBnet.journal 17, 10 (2011).
45. Nurk, S. et al. Assembling Genomes and Mini-metagenomes from Highly Chimeric Reads. in 158-170 (2013). doi:10.1007/978-3-642-37195-0_13.
46. Hyatt, D. et al. Prodigal: prokaryotic gene recognition and translation initiation site identification. BMC Bioinformatics 11, 119 (2010).
47. Steinegger, M. & Söding, J. MMseqs2 enables sensitive protein sequence searching for the analysis of massive data sets. Nat. Biotechnol. 35, 1026-1028 (2017).
48. Katoh, K. & Standley, D. M. MAFFT Multiple Sequence Alignment Software Version 7: Improvements in Performance and Usability. Mol. Biol. Evol. 30, 772-780 (2013).
49. Nguyen, L.-T., Schmidt, H. A., von Haeseler, A. & Minh, B. Q. IQ-TREE: A Fast and Effective Stochastic Algorithm for Estimating Maximum-Likelihood Phylogenies. Mol. Biol. Evol. 32, 268-274 (2015).
50. Letunic, I. & Bork, P. Interactive Tree Of Life (iTOL) v5: an online tool for phylogenetic tree display and annotation. Nucleic Acids Res. 49, W293-W296 (2021).

1. A genetically modified phage comprising a polynucleotide encoding an anti-defense system polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of said TAD1 polypeptide in a B. subtilis BEST7003 comprising SEQ ID NO: 741 increases sensitivity of said B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of said HAD1 polypeptide in a B. subtilis BEST7003 comprising SEQ ID NO: 742 increases sensitivity of said B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of said GAD1 polypeptide in a B. subtilis BEST7003 comprising SEQ ID NO: 740 increases sensitivity of said B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of said DSAD1 polypeptide in a B. subtilis BEST7003 comprising SEQ ID NO: 746 increases sensitivity of said B. subtilis BEST7003 to infection by a B. subtilis phage SPR; and
(v) a TAD2 polypeptide having at least 80% identity and/or an e-value K 0.05 to an amino acid sequence selected from the group consisting of SEQ ID NO: 1046-1295, and wherein expression of said TAD2 polypeptide in a B. subtilis BEST7003 comprising SEQ ID NO: 741 or 1300 increases sensitivity of said B. subtilis BEST7003 to infection by at least one B. subtilis phage selected from the group consisting of SPO1L1, SPO1L2, SPO1L4 and SPO1L5.

2. (canceled)

3. The genetically modified phage of claim 1, having an increased infectivity to at least one bacteria as compared to a non-genetically modified phage of the same species.

4. The genetically modified phage of claim 1, wherein said polynucleotide is heterologous to said phage.

5. The genetically modified phage of claim 1, wherein said phage comprises genomic segments of a distinct phage integrated in a genome of said phage.

6. A nucleic acid construct comprising a polynucleotide encoding an anti-defense polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of said TAD1 polypeptide in a B. subtilis BEST7003 comprising SEQ ID NO: 741 increases sensitivity of said B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of said HAD1 polypeptide in a B. subtilis BEST7003 comprising SEQ ID NO: 742 increases sensitivity of said B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of said GAD1 polypeptide in a B. subtilis BEST7003 comprising SEQ ID NO: 740 increases sensitivity of said B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of said DSAD1 polypeptide in a B. subtilis BEST7003 comprising SEQ ID NO: 746 increases sensitivity of said B. subtilis BEST7003 to infection by a B. subtilis phage SPR; and
(v) a TAD2 polypeptide having at least 80% identity and/or an e-value≤0.05 to an amino acid sequence selected from the group consisting of SEQ ID NO: 1046-1295, and wherein expression of said TAD2 polypeptide in a B. subtilis BEST7003 comprising SEQ ID NO: 741 or 1300 increases sensitivity of said B. subtilis BEST7003 to infection by at least one B. subtilis phage selected from the group consisting of SPO1L1, SPO1L2, SPO1L4 and SPO1L5;
and a nucleic acid sequence heterologous to said polynucleotide which facilitates expression and/or integration of said polynucleotide in a phage genome.

7. (canceled)

8. The nucleic acid construct of claim 6, wherein said nucleic acid sequence is selected from the group consisting of: a promoter, a recombination element, an element for expression of multiple polynucleotides from a single construct, a transmissible element and a selectable marker.

9. The genetically modified phage of claim 1, wherein said at least 80% is at least 90%.

10. The genetically modified phage of claim 1, wherein said polynucleotide comprises said SEQ ID NO.

11. The genetically modified phage claim 1, wherein:
said amino acid sequence of (i) is selected from the group consisting of SEQ ID NO: 22, 324, 337, 338, 345, 400, 404, 438, 460, 464 and 543;
said amino acid sequence of (ii) is selected from the group consisting of SEQ ID NO: 32, 598, 604, 607 and 609;
said amino acid sequence of (iii) is selected from the group consisting of SEQ ID NO: 33, 689, 723, 725 and 726; and/or
said amino acid sequence of (v) is selected from the group consisting of SEQ ID NO: 1046-1051.

12. (canceled)

13. A method of producing a phage, the method comprising contacting the phage with the nucleic acid construct of claim 6, under conditions which allow integration of said polynucleotide in a genome of said phage, thereby producing the phage.

14. The genetically modified phage claim 1, wherein said phage does not endogenously express said anti-defense system polypeptide.

15. A method of infecting a bacteria, the method comprising contacting the bacteria with the genetically modified phage of claim 1, thereby infecting the bacteria.

16. The method of claim 15, wherein said contacting is effected in-vitro or ex-vivo.

17. The method of claim 15, wherein said contacting is effected in-vivo.

18. A method of treating a bacterial infection in a subject in need thereof, the method comprising administering to the subject a therapeutically effective amount of a phage comprising a polynucleotide encoding an anti-defense system polypeptide selected from the group consisting of:
(i) a TAD1 polypeptide having at least 80% identity and/or an e-value≤0.05 to an amino acid sequence selected from the group consisting of SEQ ID NO: 22 and 323-577, and wherein expression of said TAD1 polypeptide in a B. subtilis BEST7003 comprising SEQ ID NO: 741 increases sensitivity of said B. subtilis BEST7003 to infection by at least one B. subtilis phage selected from the group consisting of SBSphiJ, SBSphiJ1, SBSphiJ2, SBSphiJ3, SBSphiJ4, SBSphiJ5 and SBSphiJ6;
(ii) a HAD1 polypeptide having at least 80% identity and/or an e-value≤0.05 to an amino acid sequence selected from the group consisting of SEQ ID NO: 32 and 595-610, and wherein expression of said HAD1 polypeptide in a B. subtilis BEST7003 comprising SEQ ID NO: 742 increases sensitivity of said B. subtilis BEST7003 to infection by at least one B. subtilis phage selected from the group consisting of SBSphiJ, SBSphiJ1, SBSphiJ2, SBSphiJ3, SBSphiJ5, SBSphiJ6 and SBSphiJ7;
(iii) a GAD1 polypeptide having at least 80% identity and/or an e-value≤0.05 to an amino acid sequence selected from the group consisting of SEQ ID NO: 33 and 675-737, and wherein expression of said GAD1 polypeptide in a B. subtilis BEST7003 comprising SEQ ID NO: 740 increases sensitivity of said B. subtilis BEST7003 to infection by at least one B. subtilis phage selected from the group consisting of SpBeta, SpBetaL6, SpBetaL7, SpBetaL8;
(iv) a DSAD1 polypeptide having at least 80% identity and/or an e-value≤0.05 to SEQ ID NO: 739, and wherein expression of said DSAD1 polypeptide in a B. subtilis BEST7003 comprising SEQ ID NO: 746 increases sensitivity of said B. subtilis BEST7003 to infection by a B. subtilis phage SPR; and
(v) a TAD2 polypeptide having at least 80% identity and/or an e-value K 0.05 to an amino acid sequence selected from the group consisting of SEQ ID NO: 1046-1295, and wherein expression of said TAD2 polypeptide in a B. subtilis BEST7003 comprising SEQ ID NO: 741 or 1300 increases sensitivity of said B. subtilis BEST7003 to infection by at least one B. subtilis phage selected from the group consisting of SPO1L1, SPO1L2, SPO1L4 and SPO1L5,
thereby treating the bacterial infection in the subject.

19. (canceled)

20. The method of claim 18, comprising administering to the subject a therapeutically effective amount of an antibiotic.

21-27. (canceled)

28. The method of claim 18, wherein said phage is the genetically modified phage of any one of claims 1-5, 9-12 and 14.

29. The method of claim 18, wherein said bacteria is selected from the group consisting of E. coli, P. aeruginosa, K. pneumoniae and C. difficile.
